(19) United States
(12) Patent Application Publication
(10) Pub. No.: US 2025/0263390 A1
(43) Pub. Date: Aug. 21, 2025

(54) NOVEL SUBSTITUTED 6,7-DIHYDRO-5H-BENZO[7]ANNULENE COMPOUNDS AND THEIR DERIVATIVES, PROCESSES FOR THEIR PREPARATION AND THERAPEUTIC USES THEREOF

(71) Applicant: SANOFI, Paris (FR)

(72) Inventors: Patrick BERNARDELLI, Paris (FR); Youssef EL-AHMAD, Paris (FR); Frédéric PETIT, Paris (FR); Franck SLOWINSKI, Paris (FR); Corinne TERRIER, Paris (FR)

(21) Appl. No.: 18/856,523

(22) PCT Filed: Apr. 14, 2023
(86) PCT No.: PCT/EP2023/059815

§ 371 Date: Oct. 11, 2024

(30) Foreign Application Priority Data
Apr. 15, 2022 (EP) 22305566.6

(51) Int. Cl. C07D 401/06; A61K 31/397; A61K 31/4427; C07D 205/04; C07D 405/10

(52) U.S. Cl. CPC C07D 401/06; A61K 31/397; A61K 31/4427; C07D 205/04; C07D 405/10

(57) ABSTRACT

Disclosed herein are compounds of the formula (I), or pharmaceutically acceptable salts thereof formula (I) wherein R1 and R2 represent a hydrogen or a deuterium atom; R3 represents a hydrogen atom, a —COOH group or a —OH group; R3′ and R3″ represent a hydrogen atom, a methyl group, a methoxy group, a chlorine atom, a fluorine atom, or a cyano group; R4 and R5 represent a hydrogen atom, a halogen atom, a —NH₂ group, a (C₁-C₃)alkyl group, a (C₁-C₃)alkoxy group, or a —OH group; or R4 and R5 together form an oxo group or R4 and R5 together form a ═NOCH3 group or a (C₃-C₅)cycloalkyl group; R7 represents a hydrogen atom, a methyl group, a —OH group or a fluorine atom; R6 represents a phenyl group, a fused phenyl group, a bicyclic group comprising 5 to 12 carbon atoms, a heteroaryl group comprising 2 to 9 carbon atoms and comprising from 1 to 3 heteroatoms, a cycloalkyl group comprising 3 to 7 carbon atoms, a (C₃-C₆)cycloalkyl(C₁-C₃)alkyl group, a 4 to 7 membered-heterocycloalkyl group, a (C₁-C₆)alkyl group or a phenyl(C₁-C₂)alkyl group; X represents —CH₂—, —O— or —S—; Y represents —CH═, —N═ or —CR″═; R8 represents a (C₁-C₃)alkyl group, a halogen atom, a cyano group, or a (C₁-C₃)fluoroalkyl group; R9 represents a hydrogen atom or a fluorine atom; R10 and R10′ represent a hydrogen atom or a fluorine atom; R11 represents a hydrogen atom a (C₁-C₃)alkyl group or a cyclopropyl group; n is 0, 1 or 2, and m is 0 or 1. Further disclosed are process for preparing the same, pharmaceutical compositions comprising them as well as said compounds of formula (I) for use as an inhibitor and degrader of estrogen receptors, in particular in the treatment of ovulatory dysfunction, cancer, endometriosis, osteoporosis, benign prostatic hypertrophy or inflammation.

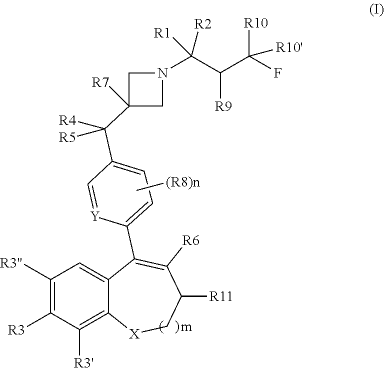

Disclosed herein are novel substituted 6,7-dihydro-5H-benzo[7]annulene derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERα-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1—blocking estrogen synthesis by aromatase inhibitors that are used to treat early and advanced ERα-positive breast cancer patients; 2—antagonizing estrogen ligand binding to ERα by tamoxifen which is used to treat ERα-positive breast cancer patients in both pre- and post-menopausal setting; 3-antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669 and WO2018/091153 disclose some substituted 6,7-dihydro-5H-benzo[7]annulene compounds and substituted N-(3-fluoropropyl)-pyrrolidine derivatives useful as SERDs.

The inventors have now found novel compounds able to selectively antagonize and degrade the estrogen receptors (SERDs compounds), for use for example in cancer treatment.

Disclosed herein are compounds of the formula (I), or pharmaceutically acceptable salts thereof:

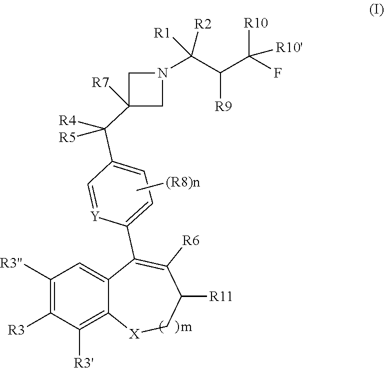

wherein:
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 and R5 independently represent a hydrogen atom, a fluorine atom, a —NH₂ group, a (C₁-C₃)alkyl group such as a methyl group, a (C₁-C₃)alkoxy group such as a methoxy group or an ethoxy group, or a —OH group; or R4 and R5 together form an oxo group or R4 and R5 together form a ═NOCH₃ group or a (C₃-C₅)cycloalkyl group with the carbon atom to which they are attached;
R7 represents a hydrogen atom, a methyl group, a —OH group or a fluorine atom;
   or alternatively R4 and R7 together form a cyclopropyl group together with the bond to which they are attached, that gives with the adjacent azetidine group an azaspiro[2.3]hexane;
R6 represents a group selected from:
   a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group; a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
   a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃) alkyl group, a hydroxy group, a halogen atom, a (C₁-C₆)fluoroalkyl group and a (C₁-C₃)alkoxy group;
   a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
   a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)-alkyl group, a (C₁-C₃)fluoroalkyl group, a (C₁-C₃)alkoxy group, a (C₁-C₃)fluoroalkoxy group and an oxo group;
   a heteroaryl group comprising 2 to 9 carbon atoms and comprising from 1 to 3 heteroatoms independently selected from oxygen, nitrogen and sulfur, and at least 5 atoms including carbon atoms and heteroatoms, such as a pyridyl group, a pyridone group or a pyrrolyl group, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
   a cycloalkyl group comprising 3 to 7 carbon atoms, said cycloalkyl group being saturated or partially saturated and being optionally substituted with 1 to 4 substituents independently selected from:
      a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
      a (C₃-C₆)cycloalkyl group, and a phenyl group, said (C₃-C₆)cycloalkyl or phenyl groups being optionally substituted with one or two halogen atom(s) or (C₁-C₃)alkyl group(s);
   a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, such as a tetrahydropyranyl or a tetrahydrofuranyl group, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
   a (C₁-C₆)alkyl group, such as an isobutyl group, a propyl group or an ethylbutyl group, said alkyl group being optionally substituted with 1 to 4 substituents independently selected from: a fluorine atom, a (C₁-C₃)alkoxy group, a (C₁-C₃)fluoroalkoxy group and a —OH group; and
   a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃) fluoroalkoxy group; a cyano group; and a —OH group;
X represents —CH₂—, —O— or —S—;
Y represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R8 independently represents a (C₁-C₃)alkyl group, such as a methyl group, a halogen atom, such as a fluorine atom, a cyano group, or a (C₁-C₃)fluoroalkyl group, such as a trifluoromethyl;
R9 represents a hydrogen atom or a fluorine atom;
R10 and R10′ independently represent a hydrogen atom or a fluorine atom;
R11 represents a hydrogen atom, or a (C₁-C₃)alkyl group or a cyclopropyl;
n is 0, 1 or 2, and
m is 0 or 1.

The compounds of formula (I) can contain one or more asymmetric carbon atoms. They may therefore exist in the form of enantiomers.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), hydrochloride may be cited.

As used herein, the terms below have the following definitions unless otherwise mentioned throughout the instant specification:
a halogen atom: a fluorine, a chlorine, a bromine or an iodine atom, and in particular a fluorine and a chlorine atom;
an oxo: a “═O” group;
an alkyl group: a linear or branched saturated hydrocarbon-based aliphatic group comprising, unless otherwise mentioned, from 1 to 6 carbon atoms (noted “(C₁-C₆)-alkyl”). By way of examples, mention may be made of, but not limited to: methyl, ethyl, propyl, isopropyl, butyl, isobutyl, sec-butyl, tert-butyl, pentyl, isopentyl, hexyl and isohexyl groups, and the like;
an alkylene group: a linear or branched hydrocarbon-based aliphatic group comprising, unless otherwise mentioned, from 1 to 6 carbon atoms (noted “(C₁-C₆)-alkylene”) and at least an unsaturation. By way of examples, mention may be made of, but not limited to: vinyl group, and the like;
a cycloalkyl group: a monocyclic alkyl group comprising, unless otherwise mentioned, from 3 to 7 carbon atoms, saturated or partially unsaturated and unsubstituted or substituted. By way of examples, mention may be made of, but not limited to: cyclopropyl, cyclobutyl, cyclopentyl, cyclobutenyl, cyclopentenyl, cyclohexyl, cyclohexenyl, cycloheptyl, cycloheptenyl, groups and the like, in particular a cyclopentyl, a cyclohexyl, a cycloheptyl, a cycloheptenyl, or a cyclohexenyl;
a cycloalkylalkyl group: an alkyl group substituted with a cyclic alkyl group as defined above. Mention may be made of, but not limited to: cyclobutylmethyl;
a heterocycloalkyl group: a 4 to 7-membered cycloalkyl group, in particular a 4 to 6-membered cycloalkyl group, saturated or partially unsaturated, comprising 1 to 2 heteroatoms independently selected from oxygen, nitrogen and sulfur, in particular being oxygen or nitrogen. By way of examples, mention may be made of, but not limited to: morpholinyl, piperazinyl, piperidinyl, pyrrolidinyl, aziridinyl, oxanyl, oxetanyl, tetrahydropyranyl, morpholinyl, tetrahydrofuranyl, oxepanyl, diazepanyl, dioxanyl, tetrahydropyranyl, and tetrahydrothiopyranyl. The heterocycloalkyl is advantageously tetrahydrofuranyl or tetrahydropyranyl.
a fluoroalkyl group: an alkyl group as previously defined where the alkyl group is substituted with at least one fluorine atom. In other terms, at least one hydrogen atom of the alkyl group is replaced by a fluorine atom. By way of example, mention may be made of —CH₂F, —CHF₂, CH₂CHF₂, —CH₂CH₂F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like;
an alkoxy group: an —O-alkyl group where the alkyl group is as previously defined. By way of examples, mention may be made of, but not limited to: methoxy, ethoxy, propoxy, isopropoxy, linear, secondary or tertiary butoxy, isobutoxy, pentoxy or hexoxy groups, and the like;
a fluoroalkoxy group: an —O-alkyl group where the alkyl group is as previously defined and where the alkyl group is substituted with at least one fluorine atom. In other terms, at least one hydrogen atom of the alkyl group is replaced by a fluorine atom. By way of example, mention may be made of —OCH₂F, —OCHF₂, —OCH₂CH₂F and the like. When all the hydrogen atoms belonging to the alkyl group are replaced by fluorine atoms, the fluoroalkoxy group can be named perfluoroalkoxy group. By way of example, mention may be made of trifluoromethoxy group and the like;
a (C₁-C₄)alkylthio group also named a (C₁-C₄)alkylsulfanyl group: a —S-alkyl group where the alkyl group is as previously defined. By way of examples, mention may be made of, but not limited to: methylthio, ethylthio, propylthio, isopropylthio, linear, secondary or tertiary butylthio, isobutylthio, and the like;
a (C₁-C₄)alkylsulfonyl group: a —SO₂-alkyl group where the alkyl group is as previously defined. By way of examples, mention may be made of, but not limited to —SO₂CH₃, —SO₂CH₂CH₃ and the like;
a (C₁-C₄)fluoroalkylthio group also named a (C₁-C₄)fluoroalkylsulfanyl group: a —S-fluoroalkyl group where the fluoroalkyl group is as previously defined. By way of examples, mention may be made of, but not limited to: fluoromethylthio, difluoromethylthio, trifluoromethylthio and the like;
a fused phenyl group: a bicyclic radical comprising from 7 to 10 carbon atoms and that contains a phenyl moiety. Said phenyl moiety may be fused to a (C₃-C₆)cycloalkyl group, i.e. the phenyl moiety may share a bond with said (C₃-C₆)cycloalkyl group. The fused phenyl group may be bound to the rest of the molecule by its phenyl moiety. It may be substituted. Examples are, but are not limited to indanyl, bicyclo[4.2.0]octa-1(6),2,4-trienyl, tetrahydronaphthalenyl and the like;
a phenyl group fused with a hetero(C₄-C₆)cycloalkyl: a bicyclic radical comprising from 7 to 10 carbon atoms and that contains a phenyl moiety. Said phenyl moiety may be fused to a hetero(C₄-C₆)cycloalkyl group, i.e. the phenyl moiety may share a bond with said hetero(C₄-C₆)cycloalkyl group. The fused phenyl group may be bound to the rest of the molecule by its phenyl moiety. It may be substituted. Examples are, but are not limited to a chromanyl group, in particular a chroman-8-yl group and the like;
a heteroaryl group: a cyclic 5 to 10-membered aromatic group containing between 2 and 9 carbon atoms and containing between 1 and 3 heteroatoms, such as nitrogen, oxygen or sulfur. Such nitrogen atom may be substituted with an oxygen atom in order to form a —N—O bond. Such —N—O bond can be in a form of a N-oxide (—N⁺—O⁻). Said heteroaryl group may be monocyclic or bicyclic. By way of examples of heteroaryl groups, mention may be made of, but not limited to: thiophene, furan, thiadiazole, thiazole, imidazole, pyridazine, triazine, pyrazine, oxadiazole, pyrazole, isothiazole, oxazole, isoxazole, pyridine, pyrimidine, benzotriazole, benzoxazole, pyrrolo[2,3-b]pyridine, benzimidazole, benzoxadiazole, benzothiazole, benzothiadiazole, benzofuran, indole, isoquinoline, indazole, benzisoxazole, benzisothiazole, pyridone groups and the like. The heteroaryl group is advantageously pyridine, pyrrole, imidazole, pyrazine, furane, thiazole, pyrazole, thiadiazole, pyridazine, pyridone and pyrimidine, and more particularly pyridine, pyridone and pyrrole;
a bicyclic group, generally comprising 5 to 12 carbon atoms, is a hydrocarbon group selected from groups comprising two rings connected through:
   a single common atom: a “spirobicyclic ring”. Such spiro bicyclic alkyl generally comprises 5 to 11 carbon atoms referring to a “spiro(C₅-C₁1)bicyclic ring”. The rings may be saturated or partially unsaturated. Such spirobicyclic ring may be unsubstituted or substituted, in particular by at least one (C₁-C₃)alkyl group such as methyl or a fluorine. By way of examples of spiro(C₅-C₁₁)bicyclic ring as for the definition of R6, mention may be made of, but not limited to: spiro[2.3]hexane, spiro[3.3]heptane, spiro[3.3]heptene, spiro[2.5]octane and 7-azaspiro[3.5]nonane. The spiro(C₅-C₁₁)bicyclic ring is advantageously spiro[3.3]heptane or spiro[3.3]heptene still for the R6 group.
   two common atoms. In that case the bicyclic group comprises 7 to 12 carbon atoms and optionally comprises 1 to 2 unsaturations. By way of examples of such bicyclic groups, mention may be made of, but not limited to: cis-1,3a,4,5,6,6a-hexahydropentalenyl group, bicyclo[3.1.0]hexan-1-yl, bicyclo[4.1.0]heptanyl and octahydropentalenyl.
   three or more common atoms. In that case the bicyclic group comprises 6 to 10 carbon atoms, such bicyclic group may be referred to as a “bridged (C₆-C₁₀)cycloalkyl” group, the rings share three or more atoms and the bridge contains at least one atom, for example 1, 2 or 3 atoms and preferentially 1 atom. By way of examples of such bridged cycloalkyl groups, mention may be made of, but not limited to bicyclo[3.2.1]octan-3-yl and bicyclo[2.2.1]heptan-2-yl.
A zwitterion means: a globally neutral molecule with a positive and a negative electrical charge and having an acidic group and a basic group.

In another embodiment, in the compounds of formula (I) as defined above, R1 and R2 are a hydrogen atom.

In another embodiment, in the compounds of formula (I) as defined above, R3 is —COOH.

In another embodiment, in the compounds of formula (I) as defined above, X represents —CH₂—.

In another embodiment, in the compounds of formula (I) as defined above, R4 and R5 represent independently from each other a hydrogen atom, a fluorine atom, a methyl group, a methoxy group, an ethoxy group, a —NH₂ group or a —OH group; or R4 and R5 together form an oxo group, a ═NOCH₃ group or a cyclopropyl group with the carbon atom to which they are attached or alternatively R4 and R7 together form a cyclopropyl group together with the bond to which they are attached, in particular both of R4 and R5 represent hydrogen atoms or a fluorine atom, or one of R4 and R5 represents a hydrogen atom and the other a fluorine atom or a —OH group, or one of R4 and R5 represents a methyl group and the other a hydroxy group or a fluorine atom, more particularly R4 and R5 both represent a hydrogen atom.

In another embodiment, in the compounds of formula (I) as defined above, R4 and R5 represent a hydrogen atom, a —NH₂ group, a methyl group, a methoxy group, an ethoxy group.

In another embodiment, in the compounds of formula (I) as defined above, R4 and R5 both represent a hydrogen atom.

In another embodiment, in the compounds of formula (I) as defined above, R7 represents a hydrogen atom, a —OH group, a methyl group or a fluorine atom, more particularly a hydrogen atom.

In another embodiment, in the compounds of formula (I) as defined above, R6 represents a phenyl group, said phenyl group being optionally substituted with 1 to 3 substituents independently selected from a chlorine atom, a fluorine atom, a hydroxy group, a methyl group, an ethyl group, a trifluoromethyl group, a 2,2,2-trifluoroethyl group, a 1,1-difluoroethyl group, a hydroxy methyl group, a 2-hydroxyethyl group, a fluoromethyl group, a difluoromethyl group, a 2,2-difluororethyl group, a methoxy group, an ethoxy group, a cyano group, a vinyl group, a cyanomethyl group, a trifluoromethylsulfonyl group, a methylsulfanyl group, a difluoromethylsulfanyl group, a methylsulfonyl group, a trifluoromethoxy group, a cyclopropyl group, and a difluoromethoxy group.

In another embodiment, in the compounds of formula (I) as defined above, R6 represents a fused phenyl group, selected from a bicyclo[4.2.0]octa-trienyl group, a tetrahydronaphthalenyl group and an indanyl group, said groups being optionally substituted with one or two fluorine atoms or R6 represents a chromanyl group.

In another embodiment, in the compounds of formula (I) as defined above, R6 represents a cycloalkyl group selected from a cyclobutyl group, a cyclohexyl group, a cyclopentyl group, a cycloheptyl group, a cycloheptenyl group and a cyclohexenyl group, said cycloalkyl group being optionally substituted with 1 to 4 substituents independently selected from:
a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group,
a (C₃-C₆)cycloalkyl group and a phenyl group, said (C₃-C₆)cycloalkyl or phenyl groups being optionally substituted with one or two halogen atom(s) or a (C₁-C₃)alkyl group, said cycloalkyl being advantageously substituted with 1 to 2 substituents independently selected from:
   a fluorine atom, a methyl group, and
   a cyclohexyl group substituted by two halogen atoms, in particular fluor atoms.

In another embodiment, in the compounds of formula (I) as defined above, R6 represents a (C₁-C₆)alkyl group selected from an ethyl, an isobutyl group and an ethylbutyl, said alkyl group being optionally substituted with 1 to 4 substituents independently selected from: a fluorine atom, a (C₁-C₃)alkoxy group, a (C₁-C₃)fluoroalkoxy group and a —OH group, and in particular optionally substituted with 1 or 3 fluorine atoms or with a —OH group.

In another embodiment, in the compounds of formula (I) as defined above, R3′ and R3″ represent a hydrogen atom.

In another embodiment, in the compounds of formula (I) as defined above, R8 independently represents a methyl group or a fluorine atom and n is 0, 1 or 2.

In another embodiment, in the compounds of formula (I) as defined above, Y represents —CH═, —C(CH₃)═, —CF═ or —N═, and in particular —CH═ or —N═.

In another embodiment, in the compounds of formula (I) as defined above, R9 represents a hydrogen atom.

In another embodiment, in the compounds of formula (I) as defined above, R10 and R10′ represent a hydrogen atom.

In another embodiment, in the compounds of formula (I) as defined above, R11 represents a hydrogen atom.

In another embodiment, in the compounds of formula (I) as defined above, m is 1.

In another embodiment, in the compounds of formula (I) as defined above, R6 represents
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl group is at least substituted by a (C₁-C₆)alkylene group, in particular a vinyl group;
a phenyl group fused with a hetero(C₄-C₆)cycloalkyl, which hetero(C₄-C₆)cycloalkyl ring optionally comprises an unsaturation and, wherein the fused phenyl is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a cycloalkyl group comprising 3 to 7 carbon atoms, said cycloalkyl group being saturated or partially saturated and being optionally substituted with 1 to 4 substituents independently selected from:
   a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
   a (C₃-C₆)cycloalkyl group, and a phenyl group, said (C₃-C₆)cycloalkyl or phenyl groups being optionally substituted with one or two halogen atom(s) or (C₁-C₃)alkyl group(s);
wherein said cycloalkyl group is at least substituted by a (C₁-C₃)alkyl group optionally substituted with a —OH group.

In another embodiment, in the compounds of formula (I) as defined above, R6 represents
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl is substituted by at least one hydroxy group, by at least a vinyl group, by at least a trifluoromethoxy group, by at least a cyclopropyl group or by at least a 1,1-difluoroethyl group;
a tetrahydronaphthalenyl group;
a cyclobutyl group; or
a hydroxypropyl group.

In another embodiment, in the compounds of formula (I), R3 is a COOH group and R6 is a phenyl group comprising two or three substitutions independently selected from a phenyl group, said phenyl group being optionally substituted with 1 to 3 substituents independently selected from a chlorine atom, a fluorine atom, a hydroxy group, a methyl group, an ethyl group, a trifluoromethyl group, a 2,2,2-trifluoroethyl group, a 1,1-difluoroethyl group, a hydroxymethyl group, a 2-hydroxyethyl group, a fluoromethyl group, a difluoromethyl group, a 2,2-difluororethyl group, a methoxy group, an ethoxy group, a cyano group, a vinyl group, a cyanomethyl group, a trifluoromethylsulfonyl group, a methylsulfanyl group, a difluoromethylsulfanyl group, a methylsulfonyl group, a trifluoromethoxy group, a cyclopropyl group, and a difluoromethoxy group, wherein said phenyl group is at least substituted by a (C₁-C₆)alkylene group, in particular a vinyl group. In such embodiment, R3′ and R3″ are in particular hydrogen atoms. Still in such embodiment, R1, R2, R4, R5, R7, R9, R10, R10′ and R11 are hydrogen atoms. In such embodiment, Y is a —CH═group and n is equal to 0. Still in such embodiment, X is a —CH₂— group. Still in such embodiment, m is 1.

Among the compounds of formula (I) described herein, mention may be made in particular of the following compounds or a pharmaceutically acceptable salt thereof, in particular hydrochloride salt thereof:
8-(2-(difluoromethoxy)-3-fluorophenyl)-9-(4-((1-(3-fluoropropyl)azetidin-3-yl)methyl)phenyl)-6,7-dihydro-5H-benzo[7]annulene-3-carboxylic acid, (1)
9-(4-((1-(3-fluoropropyl)azetidin-3-yl)methyl)phenyl)-8-(5,6,7,8-tetrahydronaphthalen-1-yl)-6,7-dihydro-5H-benzo[7]annulene-3-carboxylic acid, (2)
9-(4-((1-(3-fluoropropyl)azetidin-3-yl)methyl)phenyl)-8-(3-(hydroxymethyl)cyclopentyl)-6,7-dihydro-5H-benzo[7]annulene-3-carboxylic acid, (3)
9-(4-((1-(3-fluoropropyl)azetidin-3-yl)methyl)phenyl)-8-(2-methoxy-3-(trifluoromethyl)phenyl)-6,7-dihydro-5H-benzo[7]annulene-3-carboxylic acid, (4)
8-(3-chloro-2-(trifluoromethoxy)phenyl)-9-(4-((1-(3-fluoropropyl)azetidin-3-yl)methyl)phenyl)-6,7-dihydro-5H-benzo[7]annulene-3-carboxylic acid, (5)
9-(4-((1-(3-fluoropropyl)azetidin-3-yl)methyl)phenyl)-8-(2-methyl-3-(trifluoromethoxy)phenyl)-6,7-dihydro-5H-benzo[7]annulene-3-carboxylic acid, (6)
9-(4-((1-(3-fluoropropyl)azetidin-3-yl)methyl)phenyl)-8-(3-methoxy-2-methylphenyl)-6,7-dihydro-5H-benzo[7]annulene-3-carboxylic acid, (7)
8-(2-(difluoromethyl)-3-methoxyphenyl)-9-(4-((1-(3-fluoropropyl)azetidin-3-yl)methyl)phenyl)-6,7-dihydro-5H-benzo[7]annulene-3-carboxylic acid, (8)
8-(chroman-8-yl)-9-(4-((1-(3-fluoropropyl)azetidin-3-yl)methyl)phenyl)-6,7-dihydro-5H-benzo[7]annulene-3-carboxylic acid, (9)
8-(4,4-dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylic acid, (10)
9-(4-((1-(3-fluoropropyl)azetidin-3-yl)methyl)phenyl)-8-(3-methoxy-2-(trifluoromethyl)phenyl)-6,7-dihydro-5H-benzo[7]annulene-3-carboxylic acid, (11)
8-(chroman-5-yl)-9-(4-((1-(3-fluoropropyl)azetidin-3-yl)methyl)phenyl)-6,7-dihydro-5H-benzo[7]annulene-3-carboxylic acid, (12)
9-(5-((1-(3,3-difluoropropyl)azetidin-3-yl)methyl)-3-fluoropyridin-2-yl)-8-(4,4-dimethylcyclohexyl)-6,7-dihydro-5H-benzo[7]annulene-3-carboxylic acid, (13)
8-(3-(difluoromethoxy)-2-methylphenyl)-9-(4-((1-(3-fluoropropyl)azetidin-3-yl)methyl)phenyl)-6,7-dihydro-5H-benzo[7]annulene-3-carboxylic acid, (14)
9-(4-((1-(3-fluoropropyl)azetidin-3-yl)methyl)phenyl)-8-(3-hydroxypropyl)-6,7-dihydro-5H-benzo[7]annulene-3-carboxylic acid, 2,2,2-trifluoroacetic acid, (15)
9-(4-((1-(3-fluoropropyl)azetidin-3-yl)methyl)phenyl)-8-(2-methoxy-4-(trifluoromethyl)phenyl)-6,7-dihydro-5H-benzo[7]annulene-3-carboxylic, acid hydrochloride, (16)
9-(4-((1-(3-fluoropropyl)azetidin-3-yl)methyl)phenyl)-8-(4-methoxy-3-(trifluoromethyl)phenyl)-6,7-dihydro-5H-benzo[7]annulene-3-carboxylic acid, hydrochloride, (17)
9-(4-((1-(3-fluoropropyl)azetidin-3-yl)methyl)phenyl)-8-(2-methyl-5-(trifluoromethyl)phenyl)-6,7-dihydro-5H-benzo[7]annulene-3-carboxylic acid, (18)
9-(4-((1-(3-fluoropropyl)azetidin-3-yl)methyl)phenyl)-8-(3-methoxy-4-(trifluoromethyl)phenyl)-6,7-dihydro-5H-benzo[7]annulene-3-carboxylic acid, hydrochloride, (19)
9-(4-((1-(3-fluoropropyl)azetidin-3-yl)methyl)phenyl)-8-(5-methyl-2-(trifluoromethyl)phenyl)-6,7-dihydro-5H-benzo[7]annulene-3-carboxylic acid, hydrochloride, (20)
9-(4-((1-(3-fluoropropyl)azetidin-3-yl)methyl)phenyl)-8-(3-(hydroxymethyl)cyclobutyl)-6,7-dihydro-5H-benzo[7]annulene-3-carboxylic acid, (21)
9-(4-((1-(3-fluoropropyl)azetidin-3-yl)methyl)phenyl)-8-(3-(trifluoromethoxy)phenyl)-6,7-dihydro-5H-benzo[7]annulene-3-carboxylic acid, hydrochloride, (22)
8-(2-(difluoromethyl)-5-methylphenyl)-9-(4-((1-(3-fluoropropyl)azetidin-3-yl)methyl)phenyl)-6,7-dihydro-5H-benzo[7]annulene-3-carboxylic acid, (23)
8-(2-(difluoromethyl)-4-methoxyphenyl)-9-(4-((1-(3-fluoropropyl)azetidin-3-yl)methyl)phenyl)-6,7-dihydro-5H-benzo[7]annulene-3-carboxylic acid, (24)
9-(4-((1-(3-fluoropropyl)azetidin-3-yl)methyl)phenyl)-8-(4-methoxy-2-(trifluoromethyl)phenyl)-6,7-dihydro-5H-benzo[7]annulene-3-carboxylic acid, hydrochloride, (25)
9-(4-((1-(3-fluoropropyl)azetidin-3-yl)methyl)phenyl)-8-(3-methyl-4-(trifluoromethyl)phenyl)-6,7-dihydro-5H-benzo[7]annulene-3-carboxylic acid, (26)
9-(4-((1-(3-fluoropropyl)azetidin-3-yl)methyl)phenyl)-8-(4-(trifluoromethoxy)phenyl)-6,7-dihydro-5H-benzo[7]annulene-3-carboxylic acid, (27)
9-(4-((1-(3-fluoropropyl)azetidin-3-yl)methyl)phenyl)-8-(2-methoxy-5-(trifluoromethyl)phenyl)-6,7-dihydro-5H-benzo[7]annulene-3-carboxylic acid, hydrochloride, (28)
9-(4-((1-(3-fluoropropyl)azetidin-3-yl)methyl)phenyl)-8-(4-methyl-3-(trifluoromethyl)phenyl)-6,7-dihydro-5H-benzo[7]annulene-3-carboxylic acid, (29)
9-(4-((1-(3-fluoropropyl)azetidin-3-yl)methyl)phenyl)-8-(2-(trifluoromethoxy)phenyl)-6,7-dihydro-5H-benzo[7]annulene-3-carboxylic acid, (30)
8-(4-fluoro-2,5-dimethylphenyl)-9-(4-((1-(3-fluoropropyl)azetidin-3-yl)methyl)phenyl)-6,7-dihydro-5H-benzo[7]annulene-3-carboxylic acid, (31)
8-(2-cyclopropyl-4-(trifluoromethyl)phenyl)-9-(4-((1-(3-fluoropropyl)azetidin-3-yl)methyl)phenyl)-6,7-dihydro-5H-benzo[7]annulene-3-carboxylic acid, hydrochloride, (32)
9-(4-((1-(3-fluoropropyl)azetidin-3-yl)methyl)phenyl)-8-(2-methyl-6-(trifluoromethyl)phenyl)-6,7-dihydro-5H-benzo[7]annulene-3-carboxylic acid, (33)
9-(4-((1-(3-fluoropropyl)azetidin-3-yl)methyl)phenyl)-8-(3-methyl-5-(trifluoromethyl)phenyl)-6,7-dihydro-5H-benzo[7]annulene-3-carboxylic acid, hydrochloride, (34)
9-(4-((1-(3-fluoropropyl)azetidin-3-yl)methyl)phenyl)-8-(5-methoxy-2-(trifluoromethyl)phenyl)-6,7-dihydro-5H-benzo[7]annulene-3-carboxylic acid, (35)
8-(3-fluoro-2-vinylphenyl)-9-(4-((1-(3-fluoropropyl)azetidin-3-yl)methyl)phenyl)-6,7-dihydro-5H-benzo[7]annulene-3-carboxylic acid, (36)
8-(2-ethyl-3-fluorophenyl)-9-(4-((1-(3-fluoropropyl)azetidin-3-yl)methyl)phenyl)-6,7-dihydro-5H-benzo[7]annulene-3-carboxylic acid, (37)
8-(3-chloro-2-ethylphenyl)-9-(4-((1-(3-fluoropropyl)azetidin-3-yl)methyl)phenyl)-6,7-dihydro-5H-benzo[7]annulene-3-carboxylic acid, (38)
8-(2,4-bis(trifluoromethyl)phenyl)-9-(4-((1-(3-fluoropropyl)azetidin-3-yl)methyl)phenyl)-6,7-dihydro-5H-benzo[7]annulene-3-carboxylic acid, (39)
8-(2-chloro-6-methylphenyl)-9-(4-((1-(3-fluoropropyl)azetidin-3-yl)methyl)phenyl)-6,7-dihydro-5H-benzo[7]annulene-3-carboxylic acid, hydrochloride, (40)
8-(2-fluoro-6-methylphenyl)-9-(4-((1-(3-fluoropropyl)azetidin-3-yl)methyl)phenyl)-6,7-dihydro-5H-benzo[7]annulene-3-carboxylic acid, hydrochloride, (41)
8-(4-(difluoromethyl)-3-methylphenyl)-9-(4-((1-(3-fluoropropyl)azetidin-3-yl)methyl)phenyl)-6,7-dihydro-5H-benzo[7]annulene-3-carboxylic acid, hydrochloride, (42)
8-(2-fluoro-6-(trifluoromethoxy)phenyl)-9-(4-((1-(3-fluoropropyl)azetidin-3-yl)methyl)phenyl)-6,7-dihydro-5H-benzo[7]annulene-3-carboxylic acid, (43)
8-(2-chloro-6-methoxyphenyl)-9-(4-((1-(3-fluoropropyl)azetidin-3-yl)methyl)phenyl)-6,7-dihydro-5H-benzo[7]annulene-3-carboxylic acid, (44)
8-(2-fluoro-6-methoxyphenyl)-9-(4-((1-(3-fluoropropyl)azetidin-3-yl)methyl)phenyl)-6,7-dihydro-5H-benzo[7]annulene-3-carboxylic acid, (45)
8-(2,3-dimethoxyphenyl)-9-(4-((1-(3-fluoropropyl)azetidin-3-yl)methyl)phenyl)-6,7-dihydro-5H-benzo[7]annulene-3-carboxylic acid, (46)
8-(2,4-difluoro-3-hydroxyphenyl)-9-(4-((1-(3-fluoropropyl)azetidin-3-yl)methyl)phenyl)-6,7-dihydro-5H-benzo[7]annulene-3-carboxylic acid, (47)
9-(4-((1-(3-fluoropropyl)azetidin-3-yl)methyl)phenyl)-8-(2-methoxy-6-methylphenyl)-6,7-dihydro-5H-benzo[7]annulene-3-carboxylic acid, hydrochloride, (48)
9-(4-((1-(3-fluoropropyl)azetidin-3-yl)methyl)phenyl)-8-(2-methoxy-3-methylphenyl)-6,7-dihydro-5H-benzo[7]annulene-3-carboxylic acid, hydrochloride, (49)
8-(2-chloro-6-(trifluoromethoxy)phenyl)-9-(4-((1-(3-fluoropropyl)azetidin-3-yl)methyl)phenyl)-6,7-dihydro-5H-benzo[7]annulene-3-carboxylic acid, (50)
8-(2,6-dimethoxyphenyl)-9-(4-((1-(3-fluoropropyl)azetidin-3-yl)methyl)phenyl)-6,7-dihydro-5H-benzo[7]annulene-3-carboxylic acid, hydrochloride, (51)
9-(4-((1-(3-fluoropropyl)azetidin-3-yl)methyl)phenyl)-8-(3-hydroxy-4-(trifluoromethyl)phenyl)-6,7-dihydro-5H-benzo[7]annulene-3-carboxylic acid, (52)
8-(2-chloro-4,6-dimethylphenyl)-9-(4-((1-(3-fluoropropyl)azetidin-3-yl)methyl)phenyl)-6,7-dihydro-5H-benzo[7]annulene-3-carboxylic acid, (53)
8-(2-(difluoromethyl)-3-methylphenyl)-9-(4-((1-(3-fluoropropyl)azetidin-3-yl)methyl)phenyl)-6,7-dihydro-5H-benzo[7]annulene-3-carboxylic acid, hydrochloride, (54)
9-(4-((1-(3-fluoropropyl)azetidin-3-yl)methyl)phenyl)-8-(4-methoxy-2,6-dimethylphenyl)-6,7-dihydro-5H-benzo[7]annulene-3-carboxylic acid, (55)
9-(4-((1-(3-fluoropropyl)azetidin-3-yl)methyl)phenyl)-8-(3-hydroxy-4-methylphenyl)-6,7-dihydro-5H-benzo[7]annulene-3-carboxylic acid, (56)
8-(3-fluoro-2,4-dimethylphenyl)-9-(4-((1-(3-fluoropropyl)azetidin-3-yl)methyl)phenyl)-6,7-dihydro-5H-benzo[7]annulene-3-carboxylic acid, (57)
8-(3-chloro-2,4-dimethylphenyl)-9-(4-((1-(3-fluoropropyl)azetidin-3-yl)methyl)phenyl)-6,7-dihydro-5H-benzo[7]annulene-3-carboxylic acid, (58)
8-(2,3-bis(trifluoromethyl)phenyl)-9-(4-((1-(3-fluoropropyl)azetidin-3-yl)methyl)phenyl)-6,7-dihydro-5H-benzo[7]annulene-3-carboxylic acid, (59)
8-(2-(difluoromethyl)-5-methoxyphenyl)-9-(4-((1-(3-fluoropropyl)azetidin-3-yl)methyl)phenyl)-6,7-dihydro-5H-benzo[7]annulene-3-carboxylic acid, hydrochloride, (60)
9-(4-((1-(3-fluoropropyl)azetidin-3-yl)methyl)phenyl)-8-(3-hydroxy-2-methylphenyl)-6,7-dihydro-5H-benzo[7]annulene-3-carboxylic acid, hydrochloride, (61)
8-(2-(1,1-difluoroethyl)-4-fluorophenyl)-9-(4-((1-(3-fluoropropyl)azetidin-3-yl)methyl)phenyl)-6,7-dihydro-5H-benzo[7]annulene-3-carboxylic acid, (62)
9-(4-((1-(3-fluoropropyl)azetidin-3-yl)methyl)phenyl)-8-(2-methoxy-6-(trifluoromethyl)phenyl)-6,7-dihydro-5H-benzo[7]annulene-3-carboxylic acid, (63)
8-(3-fluoro-2-methyl-4-(trifluoromethyl)phenyl)-9-(4-((1-(3-fluoropropyl)azetidin-3-yl)methyl)phenyl)-6,7-dihydro-5H-benzo[7]annulene-3-carboxylic acid, hydrochloride, (64)
8-(3-fluoro-2-methoxy-6-methylphenyl)-9-(4-((1-(3-fluoropropyl)azetidin-3-yl)methyl)phenyl)-6,7-dihydro-5H-benzo[7]annulene-3-carboxylic acid, (65)
8-(2-(difluoromethyl)-6-methylphenyl)-9-(4-((1-(3-fluoropropyl)azetidin-3-yl)methyl)phenyl)-6,7-dihydro-5H-benzo[7]annulene-3-carboxylic acid, (66)
8-(2-ethyl-3-(trifluoromethyl)phenyl)-9-(4-((1-(3-fluoropropyl)azetidin-3-yl)methyl)phenyl)-6,7-dihydro-5H-benzo[7]annulene-3-carboxylic acid, (67)
8-(3-ethyl-2-(trifluoromethyl)phenyl)-9-(4-((1-(3-fluoropropyl)azetidin-3-yl)methyl)phenyl)-6,7-dihydro-5H-benzo[7]annulene-3-carboxylic acid, (68)
8-(2-ethyl-4-(trifluoromethyl)phenyl)-9-(4-((1-(3-fluoropropyl)azetidin-3-yl)methyl)phenyl)-6,7-dihydro-5H-benzo[7]annulene-3-carboxylic acid, (69)
8-(4-ethyl-2-(trifluoromethyl)phenyl)-9-(4-((1-(3-fluoropropyl)azetidin-3-yl)methyl)phenyl)-6,7-dihydro-5H-benzo[7]annulene-3-carboxylic acid, (70)
8-(3,4-dimethoxyphenyl)-9-(4-((1-(3-fluoropropyl)azetidin-3-yl)methyl)phenyl)-6,7-dihydro-5H-benzo[7]annulene-3-carboxylic acid, (71)
9-(4-((1-(3-fluoropropyl)azetidin-3-yl)methyl)phenyl)-8-(2-methoxy-4,6-dimethylphenyl)-6,7-dihydro-5H-benzo[7]annulene-3-carboxylic acid, (72)
8-(3-fluoro-2,6-dimethylphenyl)-9-(4-((1-(3-fluoropropyl)azetidin-3-yl)methyl)phenyl)-6,7-dihydro-5H-benzo[7]annulene-3-carboxylic acid, (73).

Another embodiment is a compound selected from the above list, or a pharmaceutically acceptable salt thereof, for use in therapy, especially as an inhibitor and degrader of estrogen receptors.

Another embodiment is a compound selected from the above list, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above list, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof.

Another embodiment is a method of treating cancer, comprising administering to a subject in need thereof, in particular a human, a therapeutically effective amount of a compound selected from the above list, or a pharmaceutically acceptable salt thereof.

Another embodiment is a pharmaceutical composition comprising as active principle an effective dose of a compound selected from the above list, or a pharmaceutically acceptable salt thereof, and also at least one pharmaceutically acceptable excipient.

The compounds of the formula (I) can be prepared by the following processes.

The compounds of the formula (I) and other related compounds having different substituents are synthesized using techniques and materials described below or otherwise known by the skilled person in the art. In addition, solvents, temperatures and other reaction conditions presented below may vary as deemed appropriate to the skilled person in the art.

General below methods for the preparation of compounds of formula (I) optionally modified by the use of appropriate reagents and conditions for the introduction of the various moieties found in the formula (I) as described below.

The following abbreviations and empirical formulae are used:
MeCN Acetonitrile
NH₄Cl Ammonium chloride
NH₄OH Ammonium hydroxide
9-BBN 9-borabicyclo[3.3.1]nonane
CO Carbon monoxide
Cs₂CO₃ Cesium carbonate
DCM Dichloromethane
DIEA Diisopropylethylamine
DMF N,N-dimethylformamide
DMSO Dimethyl sulfoxide
Dppf 1,1′-Bis(diphenylphosphino)ferrocene
EtOH Ethanol
EtOAc Ethyl acetate
H₂ Hydrogen
HCl Hydrochloric acid
HPLC High performance liquid chromatography
LiAlH₄ Lithium aluminium hydride
LiHMDS Lithium hexamethyldisilazane
MeOH Methanol
MgSO₄ Magnesium sulfate
m-CPBA Meta-chloroperbenzoic acid
MTBE Methyl tert-butyl ether
n-BuLi n-Butyllithium
Pd/C Palladium on carbon
K₂CO₃ Potassium carbonate
KHMDS Potassium hexamethyldisilazane
KOH Potassium hydroxide
NaBH₄ Sodium borohydride
NaCl Sodium chloride
NaHCO₃Sodium bicarbonate
NaH Sodium hydride
NaHMDS Sodium hexamethyldisilazane
NaOH Sodium hydroxide
Na₂SO₄ Sodium sulfate
NaHSO₃ Sodium bisulfate
SCX Strong cation exchange
Pd(dppf)Cl₂ [1,1′-Bis(diphenylphosphino)ferrocene]dichloropalladium(II)
Pd(PPh₃)₂Cl₂ bis(triphenylphosphine) palladium(II) dichloride
PhOK Potassium phenolate
SFC Supercritical Fluid Chromatography
TEA Triethylamine
TFA Trifluoroacetic acid
THF Tetrahydrofuran
PPh₃ Triphenylphosphine
RT Room temperature
Ar Argon
DABCO 1,4-diazabicyclo[2.2.2]octane

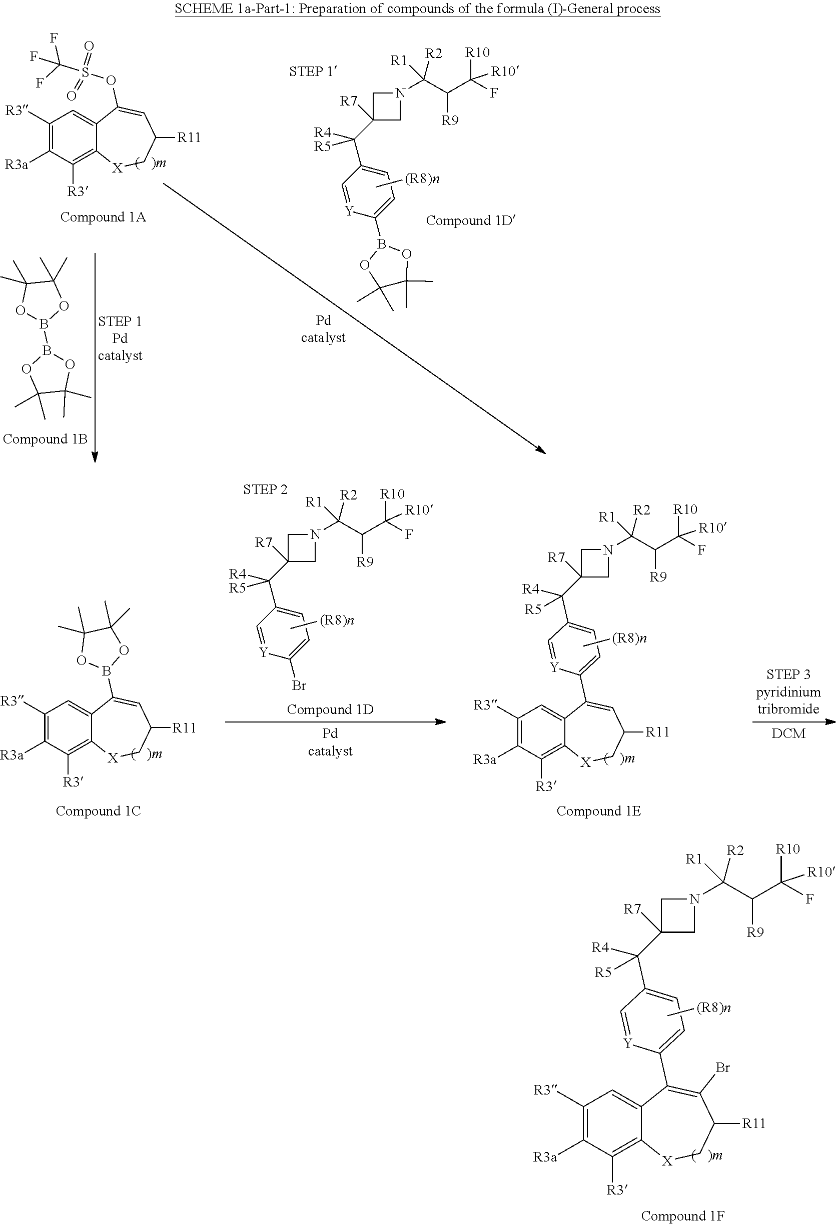

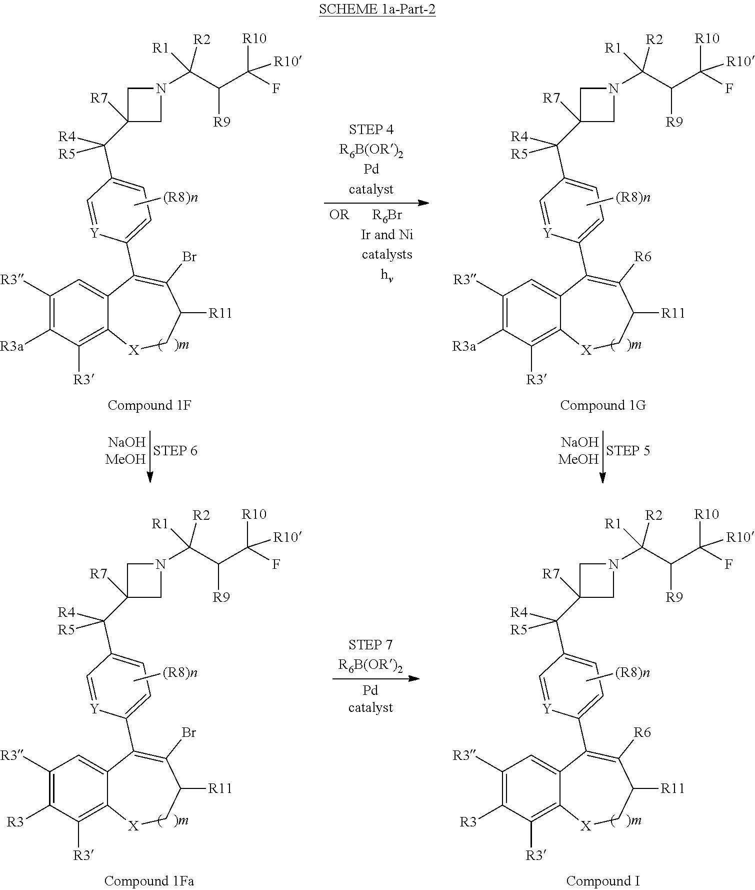

According to SCHEME 1a —Part-1 and Part-2, in which R3a is H, a carboxylic ester such as COOMe, COOEt, or protected OH such as 0-pivaloyl, R1, R2, R3, R3′, R3″, R4, R5, R6, R7, R8, R9, R10, R10′, R11, n, m, X and Y are defined as defined above, compound 1A (prepared according to WO2017140669 when X═C), can be converted in STEP 1 to compound 1C by treatment with compound 1B in the presence of a palladium catalyst, for example bis(triphenylphosphine) palladium(II) dichloride Pd(PPh₃)₂Cl₂, and a phosphine such as triphenylphosphine in solution in toluene by heating up to reflux of solvent in presence of a base such as KOPh.

Compound 1C can be converted in STEP 2 to compound 1E by treatment with compound 1D in a Suzuki coupling reaction using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Alternatively, compound 1E can be obtained in STEP 1′ by Suzuki coupling between compound 1A and compound 1D′ using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1E can be converted in STEP 3 to compound 1F by treatment for example with pyridinium tribromide in DCM or THF at room temperature.

This bromo derivative intermediate 1F can then be subjected in STEP 4 to a second Suzuki coupling with a suitable boronic reagent R6B(OR′)₂, wherein —B(OR′)₂ is a boronic acid or a pinacolate ester and R6 is defined as above, using for example Pd(dppf)Cl₂, complex with DCM, as catalyst, in a mixture of dioxane and water as solvent and in the presence of a base, for example Cs₂CO₃, at room temperature or by heating up to reflux to give compound 1G. When R6 is a substituted cycloalkene, heterocycloalkene or aliphatic ethylene, it may be reduced by hydrogenation with a catalyst such as Pd/C under hydrogen pressure (H₂) around 5 bars for example at temperature up to 70° C. to give the corresponding saturated compound 1G.

Alternatively, compound 1F can be subjected to a photocatalyzed coupling reaction with R6Br, where R6 is an alkyl group, a cycloalkyl or a spiro bicyclic alkyl as defined above, using catalysts such as (Ir[dF(CF₃)ppy]₂(dtbpy))PF₆ and nickel(II) chloride ethylene glycol dimethyl ether complex in presence of tris(trimethylsilyl)silane and bases such as 4,4′-di-tert-butyl-2,2′-bipyridine and sodium carbonate to give the corresponding compound 1G.

Compound 1G can be converted in STEP 5 to compound of formula (I) in presence of a source of hydroxide ions such as NaOH in solution in methanol (MeOH).

Intermediate 1F can be converted in STEP 6 to compound 1Fa in the presence of a source of hydroxide ions such as NaOH in solution in methanol (MeOH).

This compound 1Fa can be converted in STEP 7 to compound I through Suzuki conditions using a suitable boronic reagent R6B(OR′)₂, wherein —B(OR′)₂ is a boronic acid or a pinacolate ester and R6 is as above defined, using for example Pd(dppf)Cl₂, complex with DCM, as catalyst, in a mixture of dioxane and water as solvent and in the presence of a base, for example Cs₂CO₃, at room temperature or by heating up to reflux of solvents. When R6 is a substituted cycloalkene, heterocycloalkene or aliphatic ethylene, it may be reduced by hydrogenation with a catalyst, such as Pd/C under hydrogen (H₂) pressure around 5 bars, for example at temperature up to 70° C., to give the corresponding saturated compound I.

When R3a is COOMe, COOEt, or a protected OH such as O-pivaloyl, deprotection can be performed in STEP 5 by treatment with an aqueous solution of sodium hydroxide (NaOH) 2N or lithium hydroxide (LiOH) in MeOH. When R3 is COOH, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

Herein is also provided a process for preparing a compound of formula (I) as defined above, wherein a compound of formula 1G

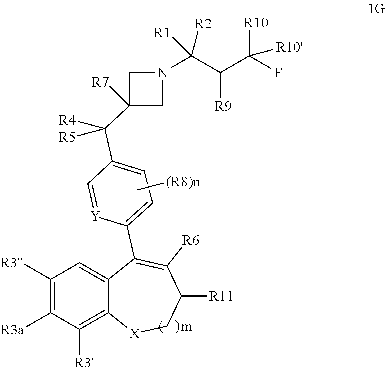

wherein R1, R2, R3′, R3″, R4, R5, R6, R7, Y, R8, R9, R10, R10′, R11, n, m, X are as defined above and R3a is carboxylic ester such as COOMe, COOEt, or protected OH such as O-pivaloyl, is converted to compound of formula (I), in presence of a source of hydroxide ions, such as NaOH in solution in methanol, said step being optionally preceded by a step for obtaining compound 1G, wherein a compound of formula 1F

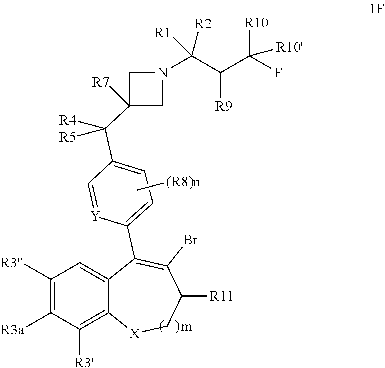

wherein, R1, R2, R3′, R3″, R4, R5, R7, Y, R8, R9, R10, R10′, R11, n, m, X are as defined above and R3a is as defined above, is subjected to a Suzuki coupling with a boronic reagent R6-B(OR′)₂, wherein —B(OR′)₂ is a boronic acid or a pinacolate ester and R6 is as defined above.

Herein is also provided a process for preparing a compound of formula (I) as defined above, wherein a compound of formula 1G

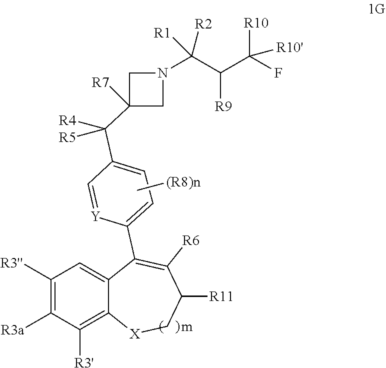

wherein R1, R2, R3′, R3″, R4, R5, R7, Y, R8, R9, R10, R10′, R11, n, m, X are as defined above,
R6 represents
   a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl group is at least substituted by a (C₁-C₆)alkylene group, in particular a vinyl group;
   a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
   a cycloalkyl group comprising 3 to 7 carbon atoms, said cycloalkyl group being saturated or partially saturated and being optionally substituted with 1 to 4 substituents independently selected from:
      a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
      a (C₃-C₆)cycloalkyl group, and a phenyl group, said (C₃-C₆)cycloalkyl or phenyl groups being optionally substituted with one or two halogen atom(s) or (C₁-C₃)alkyl group(s);
wherein said cycloalkyl group is at least substituted by a (C₁-C₃)alkyl group optionally substituted with a —OH group, and
R3a is carboxylic ester such as COOMe, COOEt, or protected OH such as O-pivaloyl, is converted to compound of formula (I), in presence of a source of hydroxide ions, such as NaOH in solution in methanol, said step being optionally preceded by a step for obtaining compound 1G, wherein a compound of formula 1F

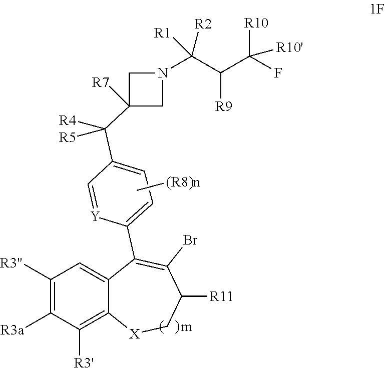

wherein, R1, R2, R3′, R3″, R4, R5, R7, Y, R8, R9, R10, R10′, R11, n, m, X are as defined above and R3a is as defined above, is subjected to a Suzuki coupling with a boronic reagent R6-B(OR′)₂, wherein —B(OR′)₂ is a boronic acid or a pinacolate ester and R6 is as defined above.

Herein is also provided a process for preparing a compound of formula (I) as defined above, wherein a compound of formula 1Fa

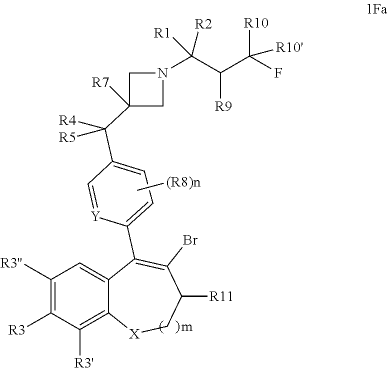

wherein R1, R2, R3, R3′, R3″, R4, R5, R7, Y, R8, R9, R10, R10′, R11, n, m, X are as defined above, is submitted to a Suzuki coupling with a boronic reagent R6-B(OR′)₂, wherein —B(OR′)₂ is a boronic acid or a pinacolate ester and R6 is defined above, said step being optionally preceded by a step for obtaining compound 1Fa, wherein a compound of formula 1F

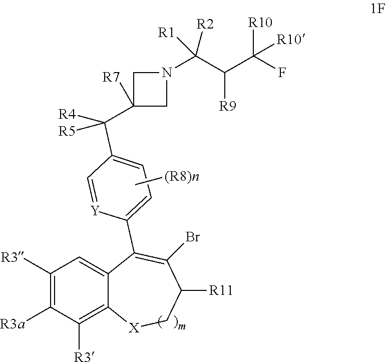

wherein R1, R2, R3′, R3″, R4, R5, R7, Y, R8, R9, R10, R10′, R11, n, m, X are as defined above and R3a is as defined above, is converted to a compound 1Fa in the presence of a source of hydroxide ions, such as NaOH in solution in methanol.

Herein is also provided a process for preparing a compound of formula (I) as defined above, wherein a compound of formula 1Fa

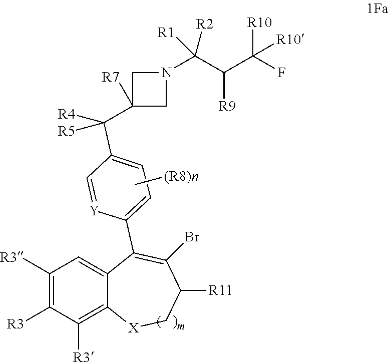

wherein R1, R2, R3, R3′, R3″, R4, R5, R7, Y, R8, R9, R10, R10′, R11, n, m, X are as defined above, is submitted to a Suzuki coupling with a boronic reagent R6-B(OR′)₂, wherein —B(OR′)₂ is a boronic acid or a pinacolate ester and R6 represents
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl group is at least substituted by a (C₁-C₆)alkylene group, in particular a vinyl group;
a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a cycloalkyl group comprising 3 to 7 carbon atoms, said cycloalkyl group being saturated or partially saturated and being optionally substituted with 1 to 4 substituents independently selected from:
   a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
   a (C₃-C₆)cycloalkyl group, and a phenyl group, said (C₃-C₆)cycloalkyl or phenyl groups being optionally substituted with one or two halogen atom(s) or (C₁-C₃)alkyl group(s);

wherein said cycloalkyl group is at least substituted by a (C₁-C₃)alkyl group optionally substituted with a —OH group, said step being optionally preceded by a step for obtaining compound 1Fa, wherein a compound of formula 1F

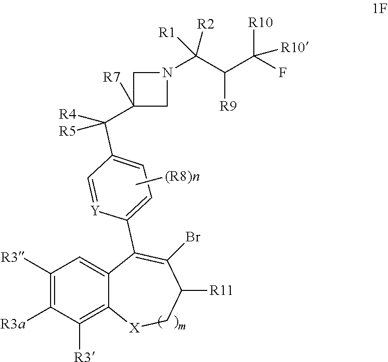

wherein R1, R2, R3′, R3″, R4, R5, R7, Y, R8, R9, R10, R10′, R11, n, m, X are as defined above and R3a is as defined above, is converted to a compound 1Fa in the presence of a source of hydroxide ions, such as NaOH in solution in methanol.

Herein are also described the intermediate compounds selected from compounds of formula 1E, 1F and 1Fa, or any of its pharmaceutically acceptable salt,

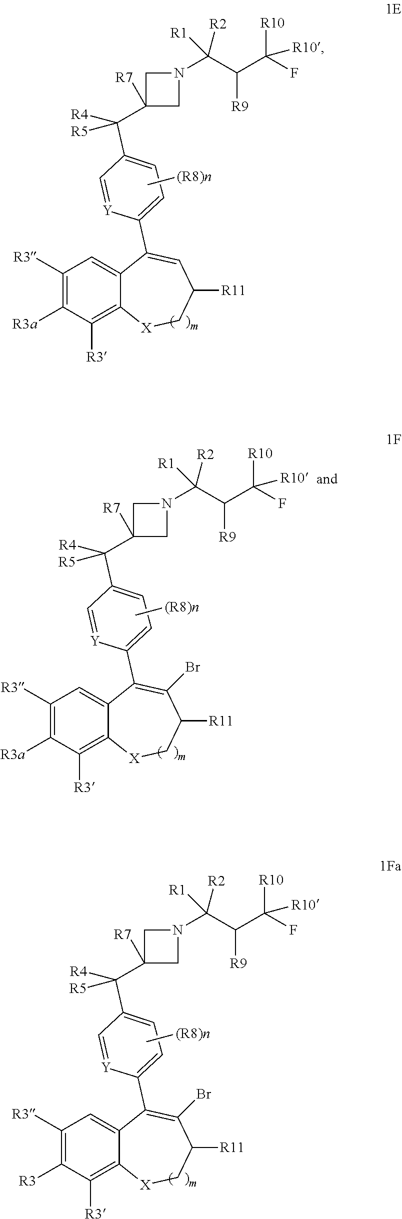

wherein R1, R2, R3, R3′, R3″, R4, R5, R7, Y, R8, R9, R10, R10′, R11, n, m, X are as defined above and R3a is carboxylic ester such as COOMe, COOEt, or protected OH such as O-pivaloyl.

Herein is also provided the intermediate compound 1G

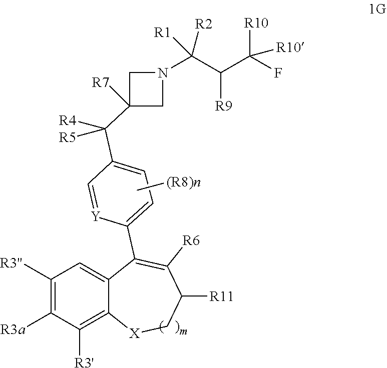

wherein R1, R2, R3′, R3″, R4, R5, R7, Y, R8, R9, R10, R10′, R11, n, m, X are as defined above, R3a is carboxylic ester such as COOMe, COOEt, or protected OH such as O-pivaloyl and R6 represents
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl group is at least substituted by a (C₁-C₆)alkylene group, in particular a vinyl group;
a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a cycloalkyl group comprising 3 to 7 carbon atoms, said cycloalkyl group being saturated or partially saturated and being optionally substituted with 1 to 4 substituents independently selected from:
   a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
   a (C₃-C₆)cycloalkyl group, and a phenyl group, said (C₃-C₆)cycloalkyl or phenyl groups being optionally substituted with one or two halogen atom(s) or (C₁-C₃)alkyl group(s);

wherein said cycloalkyl group is at least substituted by a (C₁-C₃)alkyl group optionally substituted with a —OH group.

Herein is further described the intermediate compound of formulas 1D and 1D′, or any of their pharmaceutically acceptable salt

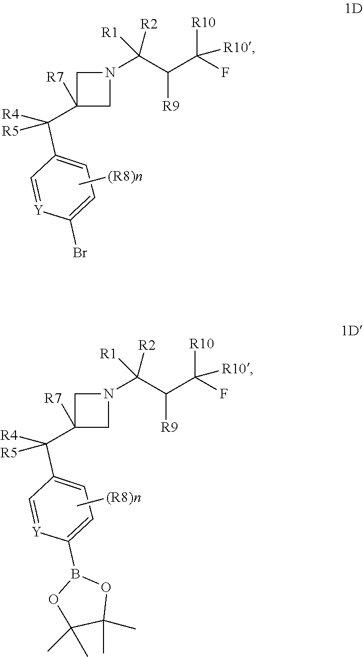

wherein R1, R2, R4, R5, R7, R8, R9, R10, R10′, n and Y are as defined above.

In another aspect, herein is also provided a process for the preparation of a compound of formula (I), wherein R3 is a —COOH group, comprising a deprotection step of a compound of formula IG as defined above, optionally followed by a purification step.

Said purification step may for example consist, as illustrated in step 2 of example 6 herein after, in an acidification step, for example with an aqueous solution of hydrochloric acid.

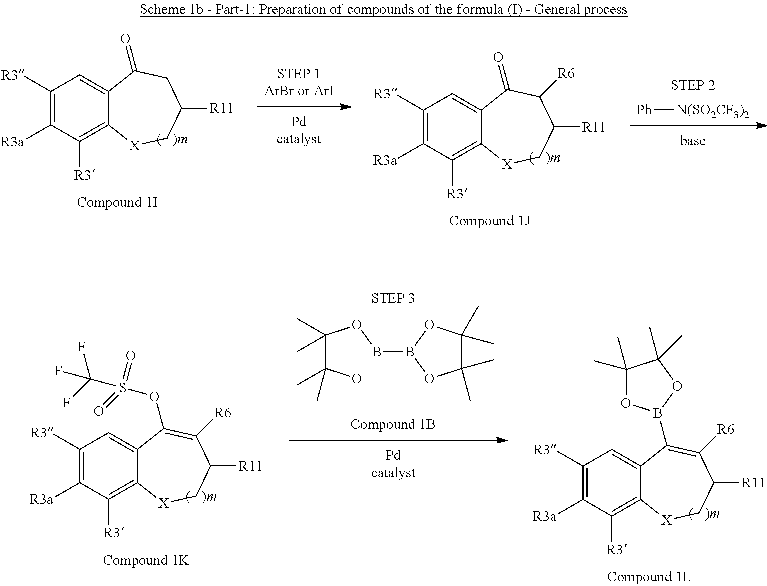

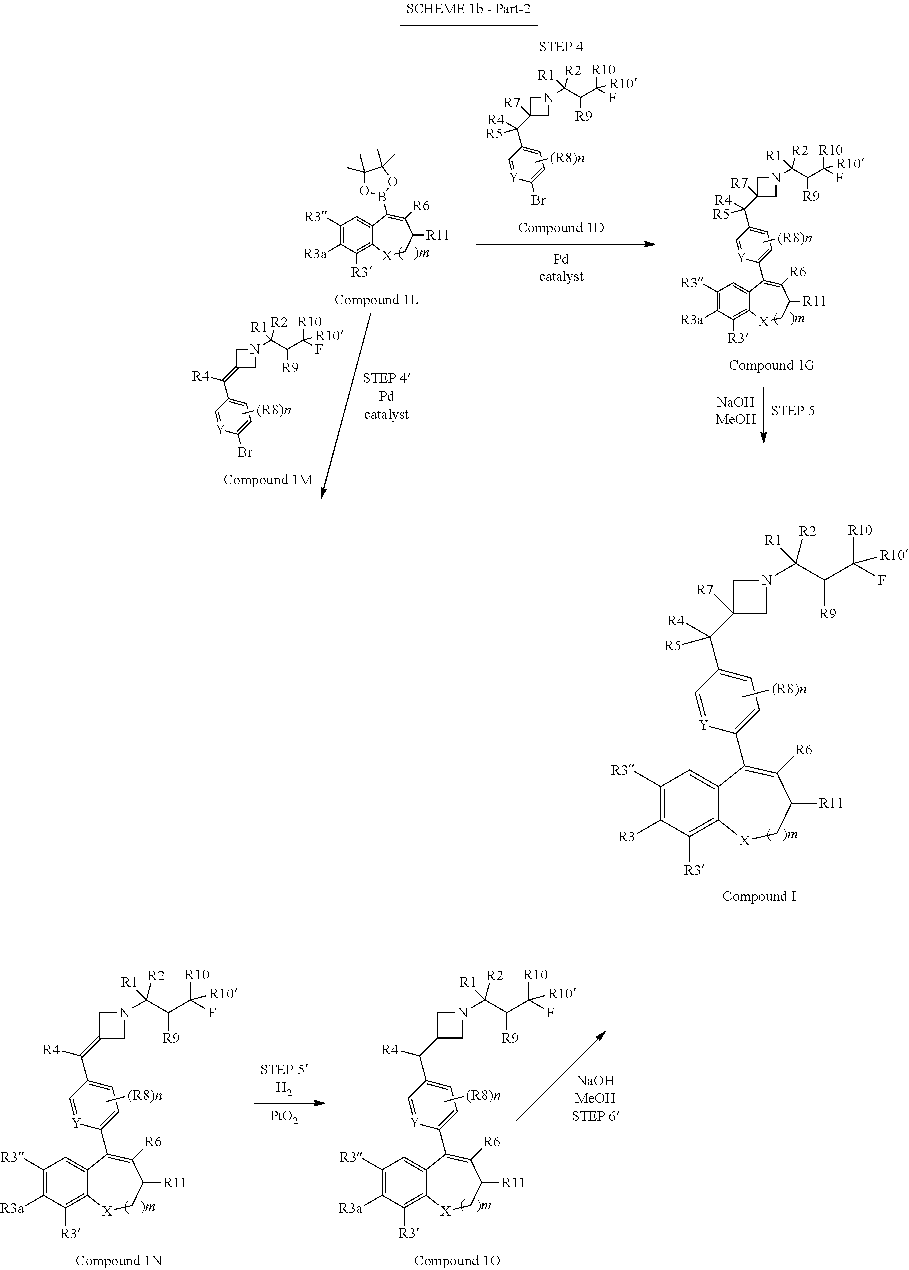

According to SCHEME 1b—Part 1 and Part 2, in which R3a is H, a carboxylic ester such as COOMe, COOEt or protected OH such as O-pivaloyl, R6 is a aryl group or a heteroaryl group, and R11 is a hydrogen atom, R1, R2, R3, R3′, R3″, R4, R5, R7, R8, R9, R10, R10′, n, m, X and Y are as defined above, compound 1I can be converted in STEP 1 to compound 1J by treatment with aryl or heteroaryl bromide or iodide in the presence of a palladium catalyst, for example tris(dibenzylideneacetone)dipalladium(0) (Pd₂(dba)₃), in solution in toluene by heating up to reflux of solvent, in presence of a base such as K₂CO₃ or Cs₂CO₃. Alternative way to prepare compound 1J, wherein R6 can be any of the groups defined above for R6 in formula (I), is described in SCHEME if below.

Compound 1J can be converted in STEP 2 to compound 1K by treatment with N,N-bis(trifluoromethylsulfonyl)aniline in the presence of base such as DBU or NaH or KHMDS at −50° C. in a solvent such as MeTHF.

Compound 1K can be converted in STEP 3 to compound 1L by treatment for example with bis(pinacolato)diboron (compound 1B), and with a palladium catalyst, for example bis(triphenylphosphine) palladium(II) dichloride Pd(PPh₃)₂Cl₂, and a phosphine such as triphenylphosphine in solution in toluene by heating up to reflux of solvent, in presence of a base such as KOPh.

Compound 1G can be prepared in a Suzuki coupling reaction between compounds 1L and 1D in STEP 4 using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

When R3a is COOMe, COOEt, or a protected OH such as O-pivaloyl, compound 1G can be converted in STEP 5 to compound of formula (I) in presence of a source of hydroxide ions such as NaOH in solution in methanol (MeOH). When R3 represents a —COOH group, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

Alternatively, compound 1L can be converted in STEP 4′ to compound 1N in a Suzuki coupling reaction with compound 1M using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1N can be reduced to compound 10 in STEP 5′ by hydrogenation with a catalyst, such as PtO₂ under hydrogen (H₂) pressure, around 2 bars for example, at room temperature.

When R3a is COOMe, COOEt, or a protected OH such as O-pivaloyl, compound 1O can be converted in STEP 6′ to compound of formula (I) in presence of a source of hydroxide ions such as NaOH in solution in methanol (MeOH). When R3 represents a —COOH group, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

Alternatively, STEPS 5′ and 6′ can be reversed to provide compound I.

Herein is further described the intermediate compound of formula 1L, or any of its pharmaceutically acceptable salt

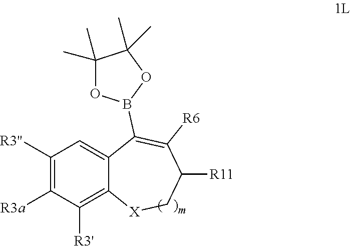

wherein R3a, R3′, R3″, X, m and R6 are as defined above and R11 is a hydrogen atom.

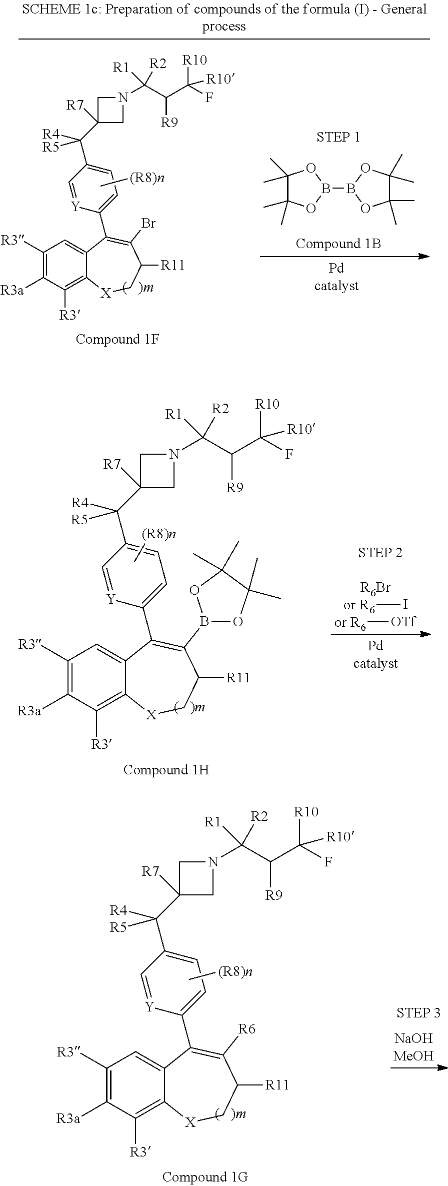

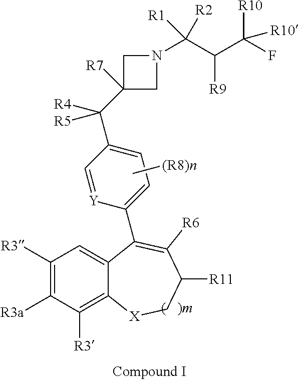

According to SCHEME 1c, in which R3a is H, a carboxylic ester such as COOMe, COOEt, or protected OH such as O-pivaloyl, and R11 is a hydrogen atom R1, R2, R3, R3′, R3″, R4, R5, R6, R7, R8, R9, R10, R10′, R11, n, m, X and Y are as defined above, compound 1F can be converted in STEP 1 to compound 1H by treatment for example with bis(pinacolato)diboron (compound 1B) and with a palladium catalyst, for example bis(triphenylphosphine)palladium(II) dichloride Pd(PPh₃)₂Cl₂, and a phosphine such as triphenylphosphine in toluene by heating up to reflux of solvent in presence of a base such as KOPh.

Compound 1G wherein R6 is phenyl or heteroaryl can be prepared in a Suzuki coupling reaction between compounds 1H and either R6Br or R6I or R6OTf in STEP 2 using for example [1,1′-bis(diphenylphosphino)ferrocene]dichloropalladium(II) (Pd(dppf)Cl₂), complex with DCM as catalyst, in a mixture of dioxane and water and in the presence of a base, for example cesium carbonate (Cs₂CO₃), by heating up to reflux of solvent.

When R3a is COOMe, COOEt, or a protected OH such as O-pivaloyl, compound 1G can be converted in STEP 3 to compound of formula (I) in presence of a source of hydroxide ions such as NaOH in solution in methanol (MeOH). When R3 represents a —COOH group, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

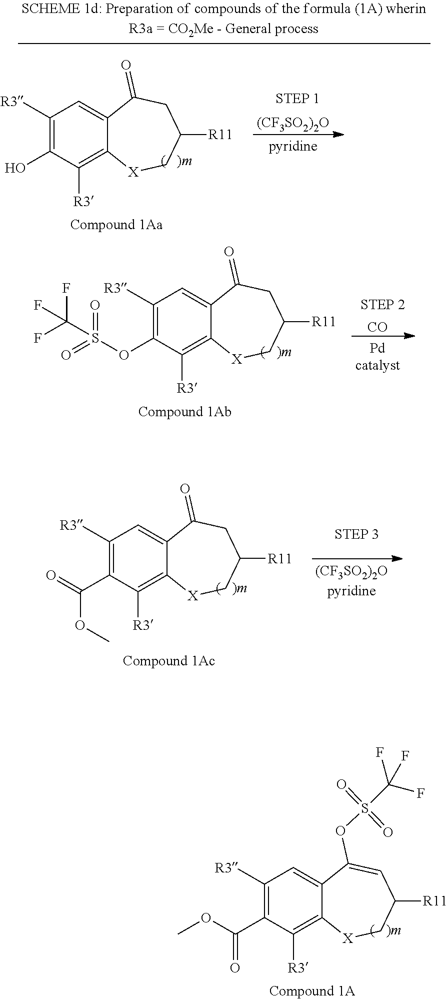

According to SCHEME 1d, in which X, m, R3′, R3″ and R11 are as defined above, compound 1A can be commercially available or prepared as follows: compound 1Aa (commercially available or prepared according to WO2017140669 and WO2018091153), can be converted in STEP 1 to compound 1Ab by treatment with trifluoromethanesulfonic anhydride, in solution in DCM, in the presence of pyridine as a base.

Compound 1Ab can be converted in STEP 2 to compound 1Ac by carbonylation with carbon monoxide, in solution in DMF and MeOH, in the presence of a palladium catalyst, for example [1,1′-bis(diphenylphosphino)ferrocene]dichloropalladium(II) (Pd(dppf)Cl₂), complex with DCM.

Compound 1Ac can be converted in STEP 3 to compound 1A wherein R3a=CO₂Me by treatment with trifluoromethanesulfonic anhydride, in solution in DCM, in the presence of pyridine as a base.

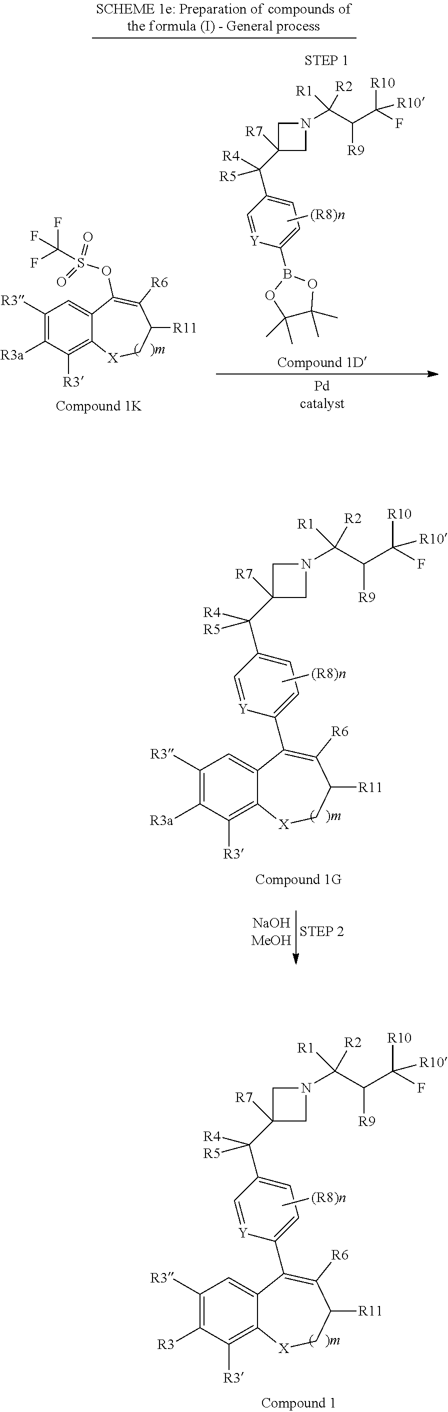

According to SCHEME 1e, in which R3a is H, a carboxylic ester such as COOMe, COOEt or protected OH such as O-pivaloyl, and R11 is a hydrogen atom R1, R2, R3, R3′, R3″, R4, R5, R6, R7, R8, R9, R10, R10′, R11, n, m, X and Y are defined as defined above, compound 1K can be converted in STEP 1 to compound 1G by treatment with compound 1D′ in the presence of a palladium catalyst, for example [1,1′-bis(diphenylphosphino)ferrocene]dichloropalladium(II) (Pd(dppf)Cl₂), complex with DCM, in a mixture of dioxane and water and in the presence of a base, for example cesium carbonate (Cs₂CO₃), by heating up to reflux of solvent.

When R3a is COOMe, COOEt, or a protected OH such as O-pivaloyl, compound 1G can be converted in STEP 2 to compound of formula (I) in presence of a source of hydroxide ions such as NaOH in solution in methanol (MeOH). When R3 represents a —COOH group, extraction of the product can give the sodium salt of compound I. The acidification with an aqueous solution of HCl 2N to pH 6-7 can give the neutral form of compound I. The acidification with an aqueous solution of HCl 2N to pH 1-2 can give the hydrochloride salt of compound I. The purification using HPLC in presence of formic acid or trifluoroacetic acid in the eluent can give the formate or trifluoroacetate salt of compound I.

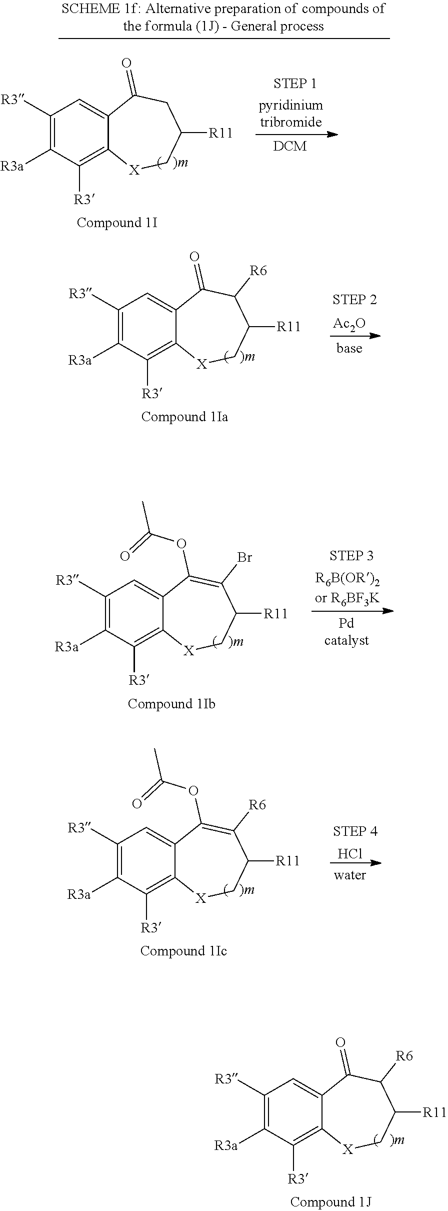

According to SCHEME 1f, in which R3a is H, a carboxylic ester such as COOMe, COOEt, or protected OH such as O-pivaloyl, R3′, R3″, R11, X and m are as defined above, compound 1J can alternatively be prepared as follows: compound 1I can be converted in STEP 1 to compound 1Ia by treatment with pyridinium tribromide in DCM or THF at room temperature for example.

Compound 1Ia can be converted in STEP 2 to compound 1Ib by deprotonation with a base such as LiHMDS in THF followed by treatment with acetic anhydride.

Compound 1Ic can be prepared in STEP 3 in a Suzuki coupling reaction between compounds 1Ib and R₆B(OR′)₂ or R₆BF₃K using for example [1,1′-bis(diphenylphosphino)ferrocene]dichloropalladium(II) (Pd(dppf)Cl₂), complex with DCM, as catalyst, in a mixture of toluene and water and in the presence of a base, for example cesium carbonate (Cs₂CO₃), by heating up to reflux of solvent.

Compound 1Ic can be converted in STEP 4 to compound 1J by hydrolysis with aqueous HCl solution by heating in methanol and DCM for example.

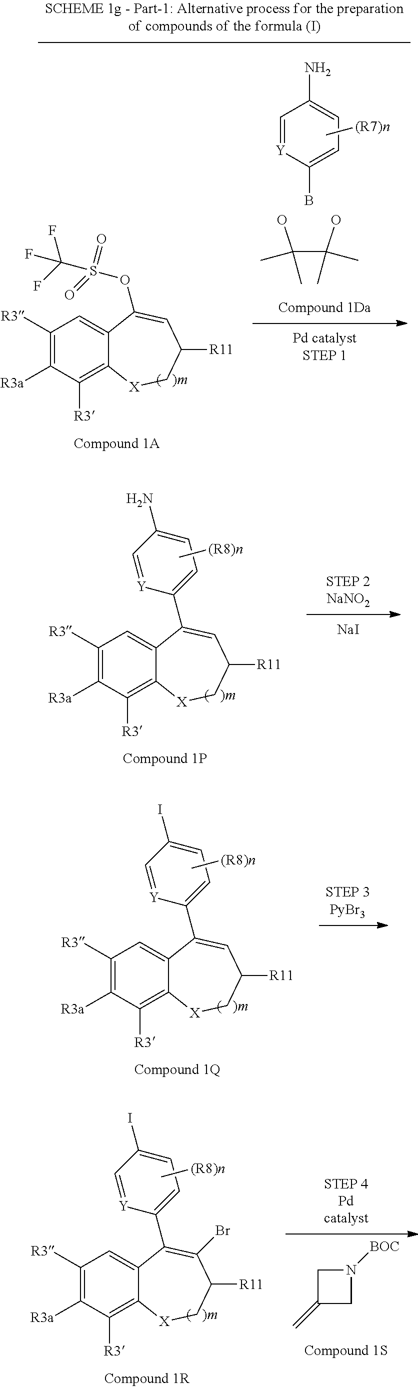

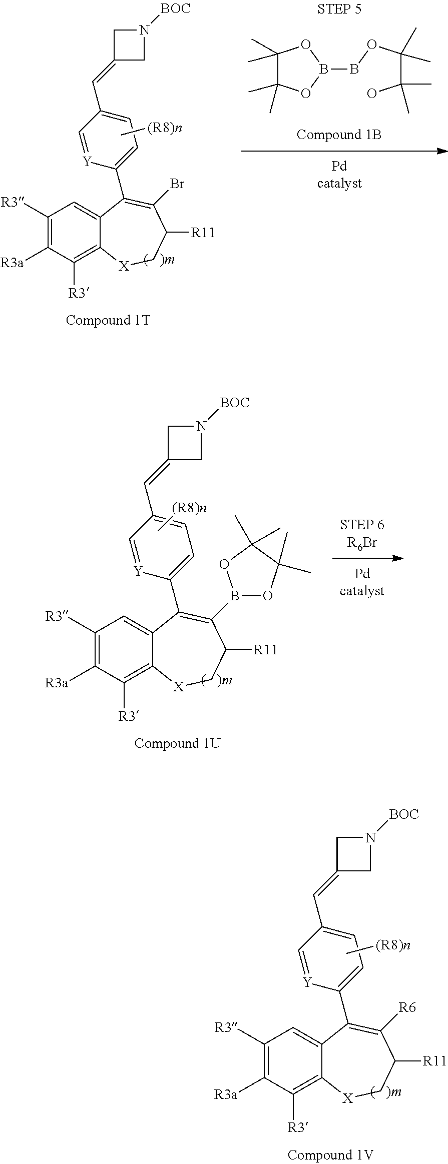

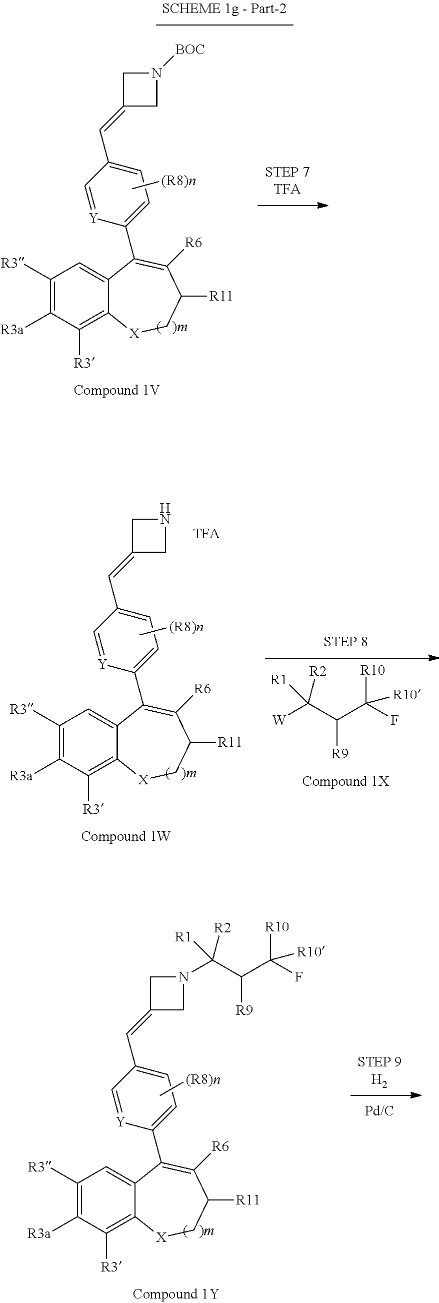

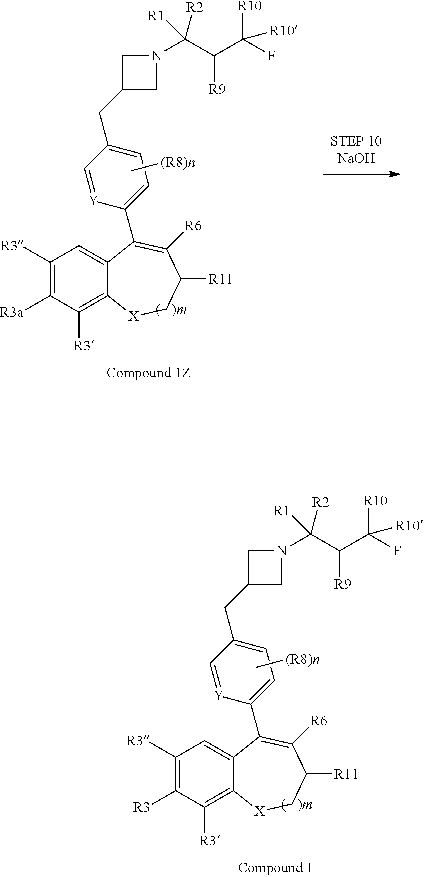

According to SCHEME 1g Part 1, in which R3a is H or a carboxylic ester such as COOMe, COOEt, or protected OH such as O-pivaloyl, and R1, R2, R3, R3′, R3″, R6, R8, R9, R10, R10′, R11, n, m, X and Y are as defined above, compound 1P can be prepared in a Suzuki coupling reaction between compounds 1A and 1 Da in STEP 1 using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

Compound 1P can be converted in STEP 2 to compound 1Q under standard Sandmeyer reaction condition such as sodium nitrite in acidic media followed by treatment sur sodium iodide. The resulting compound 1Q can be brominated in STEP 3 to compound 1R for example with pyridinium tribromide in DCM or THF at room temperature. A Heck coupling in STEP 4 by heating compound 1R with compound 1S catalyzed, for example, by palladium(II) acetate in presence of tetrabutylammonium bromide and a base such as K₂CO₃ in a solvent such as DMF can give compound 1T.

Compound 1T can be converted in STEP 5 to compound 1U by treatment for example with bis(pinacolato)diboron, and with a palladium catalyst, for example bis (triphenylphosphine)palladium(II) dichloride Pd(PPh₃)₂Cl₂, and a phosphine, such as triphenylphosphine, in solution in toluene by heating up to reflux of solvent, in presence of a base such as KOPh.

Compound 1U can be converted in STEP 6 to compound 1V in a Suzuki coupling reaction with an aryl or heteroaryl bromide or iodide using for example [1,1′-bis(diphenylphosphino)ferrocene]dichloropalladium(II) (Pd(dppf)Cl₂), complex with DCM, as catalyst, in a mixture of dioxane and water and in the presence of a base, for example cesium carbonate (Cs₂CO₃), by heating up to reflux of solvent.

According to SCHEME 1g Part 2, compound 1V can be converted in STEP 7 to compound 1W by treatment with TFA in solution in DCM or HCl in solution in dioxane.

Compound 1W can be converted in STEP 8 to compound 1Y by treatment with compound 1X, wherein W is Cl, Br or I or OSO₂R with R═CH₃, PhMe, CF₃ or CF₂CF₂CF₂CF₃, in presence of a base such as potassium carbonate in DMF at 70° C. or in presence of sodium hydroxide or potassium hydroxide in THF at room temperature or in presence of aqueous sodium hydroxide in DCM at room temperature or in presence of DIEA in MeCN at room temperature.

Compound 1Y can be converted in STEP 9 to compound 1Z by hydrogenation with a catalyst such as Pd/C under hydrogen pressure (H₂) around 5 bars for example.

When R3a is COOMe, COOEt, or a protected OH such as O-pivaloyl, compound 1Z can be deprotected into compound I in STEPS 10 by treating with an aqueous solution of sodium hydroxide (NaOH) or lithium hydroxide (LiOH), in MeOH. When R3 is —COOH, extraction of compound can give the sodium salt of compound I. The acidification with an aqueous solution of HCl 2N to pH 6-7 can give the neutral form. The acidification with an aqueous solution of HCl 2N to pH 1-2 can give the hydrochloride salt. The purification using HPLC can give the formate or trifluoroacetate salt.

Alternatively, STEPS 9 and 10 can be reversed to provide compound I.

Herein is also provided a process for preparing a compound of formula (I) as defined above, wherein a compound of formula 1Z

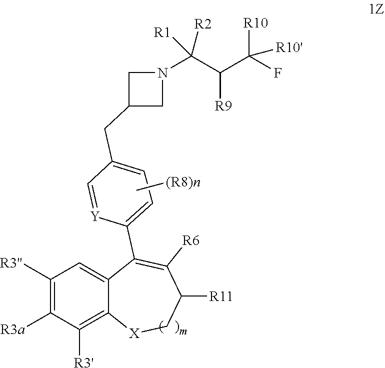

wherein R1, R2, R3′, R3″, Y, R6, R8, R9, R10, R10′, R11, n, m, X are as defined above and R3a is carboxylic ester such as COOMe, COOEt, or protected OH such as O-pivaloyl, is converted into compound of formula (I), in presence of a source of hydroxide ions, such as NaOH in solution in methanol, said step being preceded by a step for obtaining a compound 1Z, wherein compound of formula 1Y

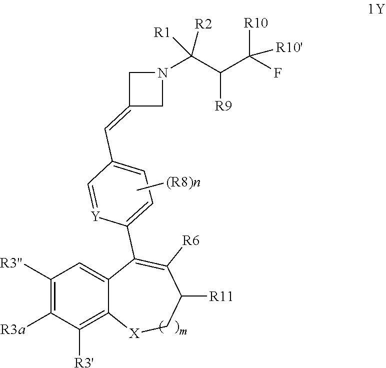

wherein R1, R2, R3, R3′, R3″, R4, R5, R6, Y, R8, R9, R10, R10′, R11, n, m, X are as defined above and R3a is carboxylic ester such as COOMe, COOEt, or protected OH such as O-pivaloyl, is converted to compound 1Z by hydrogenation with a catalyst such as Pd/C under hydrogen pressure.

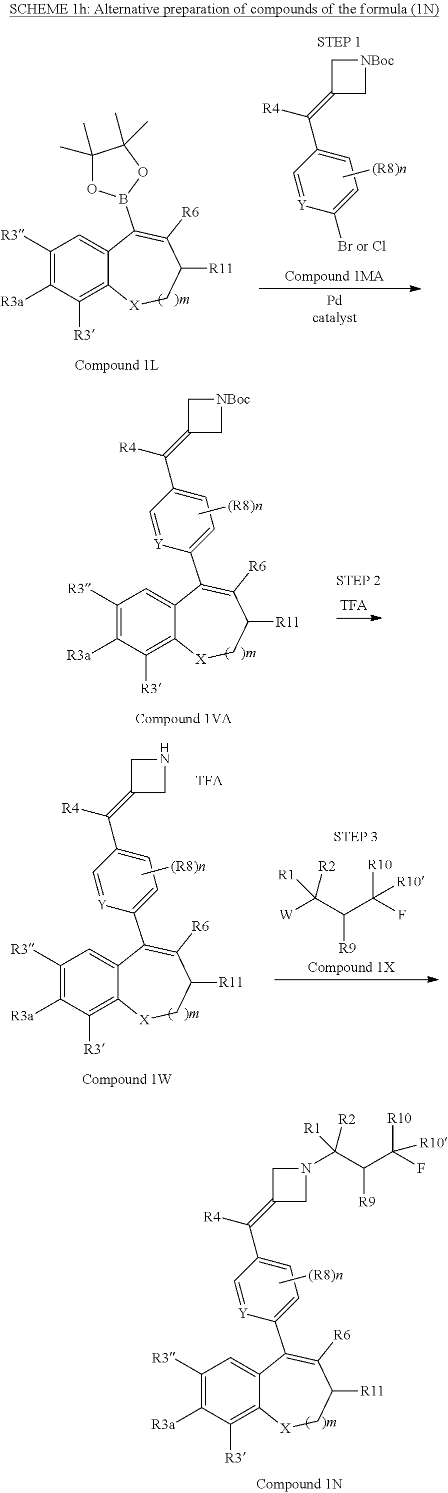

According to SCHEME 1 h, in which R3a is H, a carboxylic ester such as COOMe, COOEt or protected OH such as 0-pivaloyl, and R11 is a hydrogen atom, R1, R2, R3, R3′, R3″, R4, R6, R8, R9, R10, R10′, n, m, X and Y are defined as defined above, compound 1L can be converted in STEP 1 to compound 1VA by treatment with compound 1MA in the presence of a palladium catalyst, for example CataCXium A Pd G3 or tris(dibenzylideneacetone)dipalladium(0) (Pd₂(dba)₃), in solution in toluene by heating up to reflux of solvent, in presence of a base such as K₂CO₃ or Cs₂CO₃.

Compound 1VA can be converted in STEP 2 to compound 1W by treatment with TFA in solution in DCM or HCl in solution in dioxane.

Compound 1W can be converted in STEP 3 to compound 1N by treatment with compound 1X, wherein W is Cl, Br or I or OSO₂R with R═CH₃, PhMe, CF₃ or CF₂CF₂CF₂CF₃, in presence of a base such as potassium carbonate in DMF at 70° C. or in presence of sodium hydroxide or potassium hydroxide in THF at room temperature or in presence of aqueous sodium hydroxide in DCM at room temperature or in presence of DIEA in MeCN at room temperature.

The ¹H NMR Spectra at 400 and 500 MHz were performed on a Bruker Avance DRX-400 and Bruker Avance DPX-500 spectrometer, respectively, with the chemical shifts (6 in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HCO₂H.

The following tables 1a and 1b comprise respectively specific compounds of formula (I) (name and structure) in accordance with the present disclosure as well their characterization (¹H NMR and liquid chromatography/mass).

TABLE la
(the first column “Ex” corresponds to the compound and example number)

| Ex | Structure | Name |
|---|---|---|
| 1 | 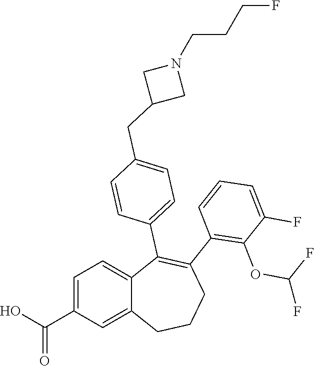 [SMILES: O=C(O)c1ccc2c(c1)CCCC(c1cccc(F)c1OC(F)F)=C2c1ccc(CC2CN(CCCF)C2)cc1] | 8-(2-(difluoromethoxy)- 3-fluorophenyl)-9-(4- ((1-(3- fluoropropyl)azetidin-3- yl)methyl)phenyl)-6,7- dihydro-5H- benzo[7]annulene-3- carboxylic acid |
| 2 | 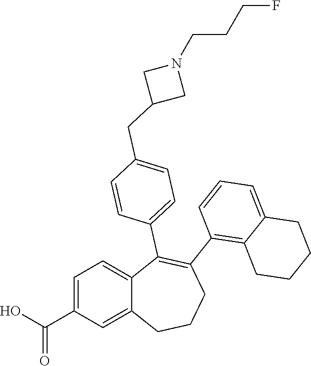 [SMILES: O=C(O)c1ccc2c(c1)CCCC(c1cccc3c1CCCC3)=C2c1ccc(CC2CN(CCCF)C2)cc1] | 9-(4-((1-(3- fluoropropyl)azetidin-3- yl)methyl)phenyl)-8- (5,6,7,8- tetrahydronaphthalen-1- yl)-6,7-dihydro-5H- benzo[7]annulene-3- carboxylic acid |
| 3 | 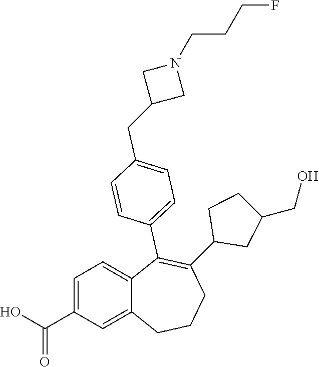 [SMILES: O=C(O)c1ccc2c(c1)CCCC(C1CCC(CO)C1)=C2c1ccc(CC2CN(CCCF)C2)cc1] | 9-(4-((1-(3- fluoropropyl)azetidin-3- yl)methyl)phenyl)-8-(3- (hydroxymethyl)cyclo- pentyl)-6,7-dihydro-5H- benzo[7]annulene-3- carboxylic acid |
| 4 | 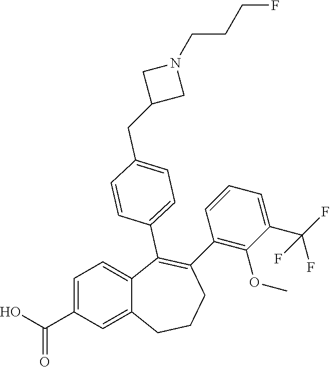 [SMILES: COc1c(C2=C(c3ccc(CC4CN(CCCF)C4)cc3)c3ccc(C(=O)O)cc3CCC2)cccc1C(F)(F)F] | 9-(4-((1-(3- fluoropropyl)azetidin-3- yl)methyl)phenyl)-8-(2- methoxy-3- (trifluoromethyl)phenyl)- 6,7-dihydro-5H- benzo[7]annulene-3- carboxylic acid |
| 5 | 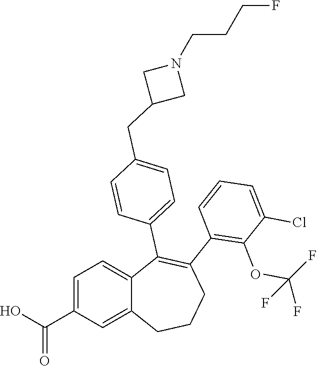 [SMILES: O=C(O)c1ccc2c(c1)CCCC(c1cccc(Cl)c1OC(F)(F)F)=C2c1ccc(CC2CN(CCCF)C2)cc1] | 8-(3-chloro-2- (trifluoromethoxy)phenyl)- 9-(4-((1-(3- fluoropropyl)azetidin-3- yl)methyl)phenyl)-6,7- dihydro-5H- benzo[7]annulene-3- carboxylic acid |
| 6 | 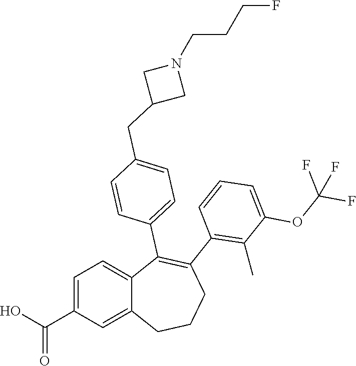 [SMILES: Cc1c(OC(F)(F)F)cccc1C1=C(c2ccc(CC3CN(CCCF)C3)cc2)c2ccc(C(=O)O)cc2CCC1] | 9-(4-((1-(3- fluoropropyl)azetidin-3- yl)methyl)phenyl)-8-(2- methyl-3- (trifluoromethoxy)phenyl)- 6,7-dihydro-5H- benzo[7]annulene-3- carboxylic acid |
| 7 | 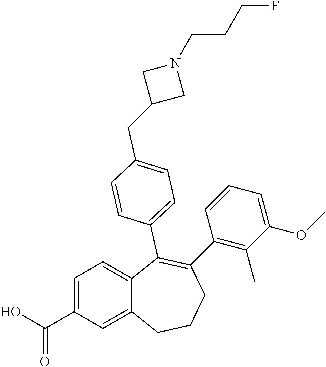 [SMILES: COc1cccc(C2=C(c3ccc(CC4CN(CCCF)C4)cc3)c3ccc(C(=O)O)cc3CCC2)c1C] | 9-(4-((1-(3- fluoropropyl)azetidin-3- yl)methyl)phenyl)-8-(3- methoxy-2- methylphenyl)-6,7- dihydro-5H- benzo[7]annulene-3- carboxylic acid |
| 8 | 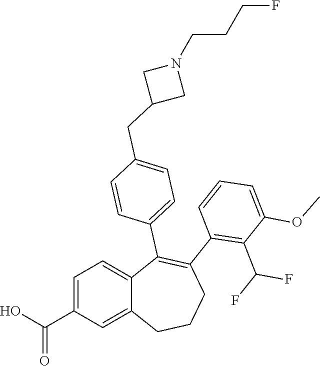 [SMILES: COc1cccc(C2=C(c3ccc(CC4CN(CCCF)C4)cc3)c3ccc(C(=O)O)cc3CCC2)c1C(F)F] | 8-(2-(difluoromethyl)-3- methoxyphenyl)-9-(4- ((1-(3- fluoropropyl)azetidin-3- yl)methyl)phenyl)-6,7- dihydro-5H- benzo[7]annulene-3- carboxylic acid |
| 9 | 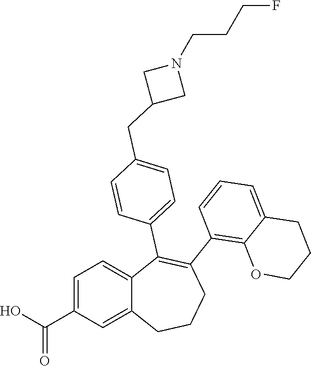 [SMILES: O=C(O)c1ccc2c(c1)CCCC(c1cccc3c1OCCC3)=C2c1ccc(CC2CN(CCCF)C2)cc1] | 8-(chroman-8-yl)-9-(4- ((1-(3- fluoropropyl)azetidin-3- yl)methyl)phenyl)-6,7- dihydro-5H- benzo[7]annulene-3- carboxylic acid |
| 10 | 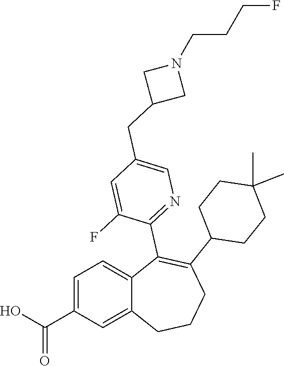 [SMILES: CC1(C)CCC(C2=C(c3ncc(CC4CN(CCCF)C4)cc3F)c3ccc(C(=O)O)cc3CCC2)CC1] | 8-(4,4- dimethylcyclohexyl)-9- (3-fluoro-5-((1-(3- fluoropropyl)azetidin-3- yl)methyl)pyridin-2-yl)- 6,7-dihydro-5H- benzo[7]annulene-3- carboxylic acid |
| 11 | 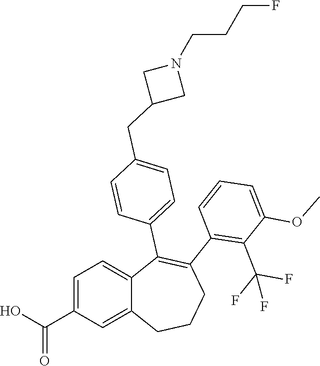 [SMILES: COc1cccc(C2=C(c3ccc(CC4CN(CCCF)C4)cc3)c3ccc(C(=O)O)cc3CCC2)c1C(F)(F)F] | 9-(4-((1-(3- fluoropropyl)azetidin-3- yl)methyl)phenyl)-8-(3- methoxy-2- (trifluoromethyl)phenyl)- 6,7-dihydro-5H- benzo[7]annulene-3- carboxylic acid |
| 12 | 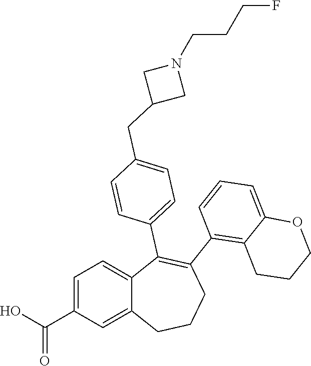 [SMILES: O=C(O)c1ccc2c(c1)CCCC(c1cccc3c1CCCO3)=C2c1ccc(CC2CN(CCCF)C2)cc1] | 8-(chroman-5-yl)-9-(4- ((1-(3- fluoropropyl)azetidin-3- yl)methyl)phenyl)-6,7- dihydro-5H- benzo[7]annulene-3- carboxylic acid |
| 13 | 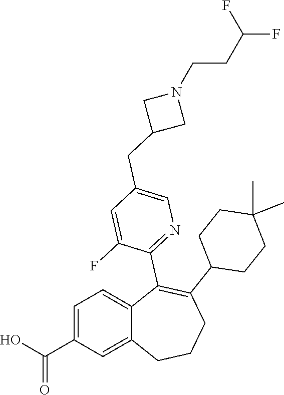 [SMILES: CC1(C)CCC(C2=C(c3ncc(CC4CN(CCC(F)F)C4)cc3F)c3ccc(C(=O)O)cc3CCC2)CC1] | 9-(5-((1-(3,3- difluoropropyl)azetidin- 3-yl)methyl)-3- fluoropyridin-2-yl)-8- (4,4- dimethylcyclohexyl)- 6,7-dihydro-5H- benzo[7]annulene-3- carboxylic acid |
| 14 | 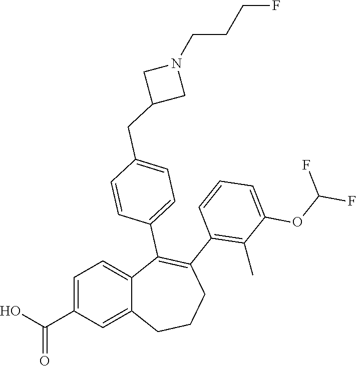 [SMILES: Cc1c(OC(F)F)cccc1C1=C(c2ccc(CC3CN(CCCF)C3)cc2)c2ccc(C(=O)O)cc2CCC1] | 8-(3-(difluoromethoxy)- 2-methylphenyl)-9-(4- ((1-(3- fluoropropyl)azetidin-3- yl)methyl)phenyl)-6,7- dihydro-5H- benzo[7]annulene-3- carboxylic acid |
| 15 | 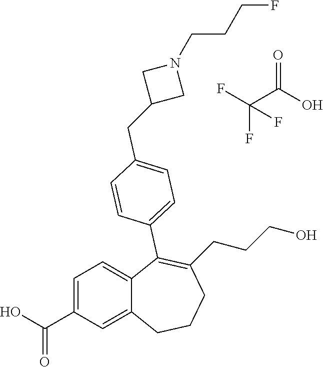 | 9-(4-((1-(3- fluoropropyl)azetidin-3- yl)methyl)phenyl)-8-(3- hydroxypropyl)-6,7- dihydro-5H- benzo[7]annulene-3- carboxylic acid, 2,2,2- 2,2,2-trifluoroacetic acid |
| 16 | 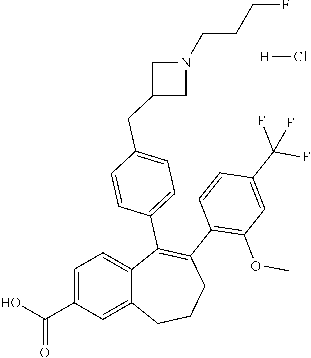 | 9-(4-((1-(3- fluoropropyl)azetidin-3- yl)methyl)phenyl)-8-(2- methoxy-4- (trifluoromethyl)phenyl)- 6,7-dihydro-5H- benzo[7]annulene-3- carboxylic acid, hydrochloride |
| 17 | 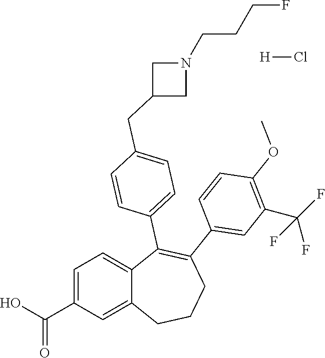 | 9-(4-((1-(3- fluoropropyl)azetidin-3- yl)methyl)phenyl)-8-(4- methoxy-3- (trifluoromethyl)phenyl)- 6,7-dihydro-5H- benzo[7]annulene-3- carboxylic acid, hydrochloride |
| 18 | 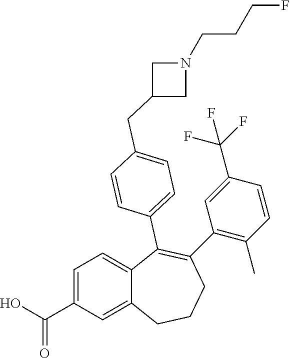 [SMILES: Cc1ccc(C(F)(F)F)cc1C1=C(c2ccc(CC3CN(CCCF)C3)cc2)c2ccc(C(=O)O)cc2CCC1] | 9-(4-((1-(3- fluoropropyl)azetidin-3- yl)methyl)phenyl)-8-(2- methyl-5- (trifluoromethyl)phenyl)- 6,7-dihydro-5H- benzo[7]annulene-3- carboxylic acid |
| 19 | 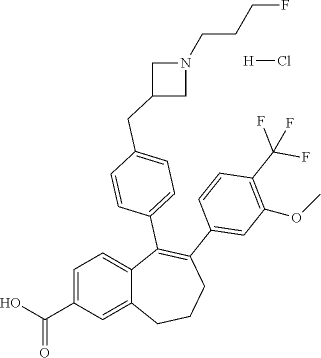 | 9-(4-((1-(3- fluoropropyl)azetidin-3- yl)methyl)phenyl)-8-(3- methoxy-4- (trifluoromethyl)phenyl)- 6,7-dihydro-5H- benzo[7]annulene-3- carboxylic acid, hydrochloride |
| 20 | 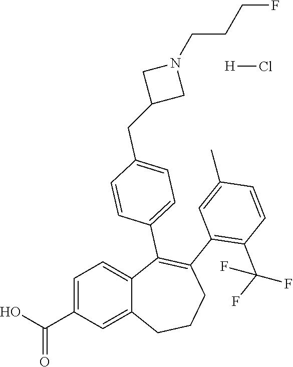 | 9-(4-((1-(3- fluoropropyl)azetidin-3- yl)methyl)phenyl)-8-(5- methyl-2- (trifluoromethyl)phenyl)- 6,7-dihydro-5H- benzo[7]annulene-3- carboxylic acid, hydrochloride |
| 21 | 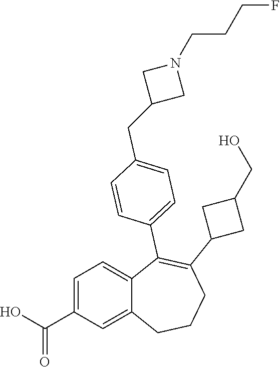 [SMILES: O=C(O)c1ccc2c(c1)CCCC(C1CC(CO)C1)=C2c1ccc(CC2CN(CCCF)C2)cc1] | 9-(4-((1-(3- fluoropropyl)azetidin-3- yl)methyl)phenyl)-8-(3- (hydroxymethyl)cyclo- butyl)-6,7-dihydro-5H- benzo[7]annulene-3- carboxylic acid |
| 22 | 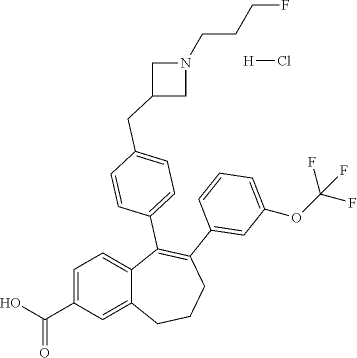 | 9-(4-((1-(3- fluoropropyl)azetidin-3- yl)methyl)phenyl)-8-(3- (trifluoromethoxy)phenyl)- 6,7-dihydro-5H- benzo[7]annulene-3- carboxylic acid, hydrochloride |
| 23 | 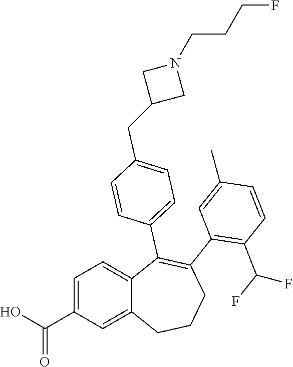 [SMILES: Cc1ccc(C(F)F)c(C2=C(c3ccc(CC4CN(CCCF)C4)cc3)c3ccc(C(=O)O)cc3CCC2)c1] | 8-(2-(difluoromethyl)-5- methylphenyl)-9-(4-((1- (3- fluoropropyl)azetidin-3- yl)methyl)phenyl)-6,7- dihydro-5H- benzo[7]annulene-3- carboxylic acid |
| 24 | 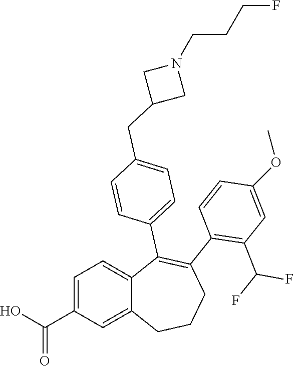 [SMILES: COc1ccc(C2=C(c3ccc(CC4CN(CCCF)C4)cc3)c3ccc(C(=O)O)cc3CCC2)c(C(F)F)c1] | 8-(2-(difluoromethyl)-4- methoxyphenyl)-9-(4- ((1-(3- fluoropropyl)azetidin-3- yl)methyl)phenyl)-6,7- dihydro-5H- benzo[7]annulene-3- carboxylic acid |
| 25 | 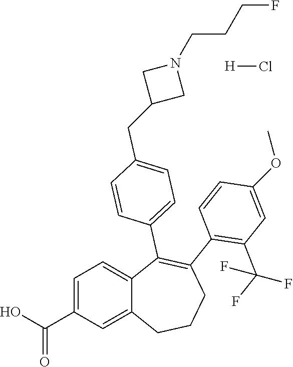 | 9-(4-((1-(3- fluoropropyl)azetidin-3- yl)methyl)phenyl)-8-(4- methoxy-2- (trifluoromethyl)phenyl)- 6,7-dihydro-5H- benzo[7]annulene-3- carboxylic acid, hydrochloride |
| 26 | 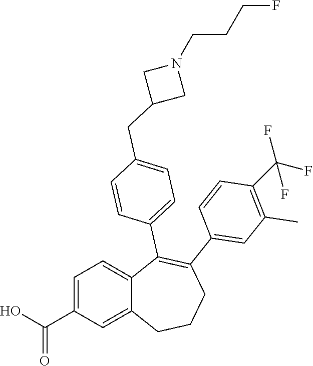 [SMILES: Cc1cc(C2=C(c3ccc(CC4CN(CCCF)C4)cc3)c3ccc(C(=O)O)cc3CCC2)ccc1C(F)(F)F] | 9-(4-((1-(3- fluoropropyl)azetidin-3- yl)methyl)phenyl)-8-(3- methyl-4- (trifluoromethyl)phenyl)- 6,7-dihydro-5H- benzo[7]annulene-3- carboxylic acid |
| 27 | 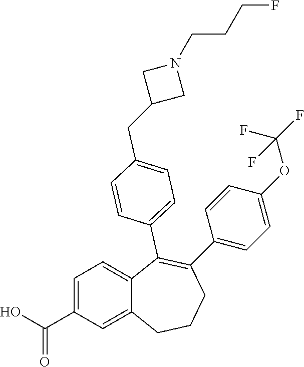 [SMILES: O=C(O)c1ccc2c(c1)CCCC(c1ccc(OC(F)(F)F)cc1)=C2c1ccc(CC2CN(CCCF)C2)cc1] | 9-(4-((1-(3- fluoropropyl)azetidin-3- yl)methyl)phenyl)-8-(4- (trifluoromethoxy)phenyl)- 6,7-dihydro-5H- benzo[7]annulene-3- carboxylic acid |
| 28 | 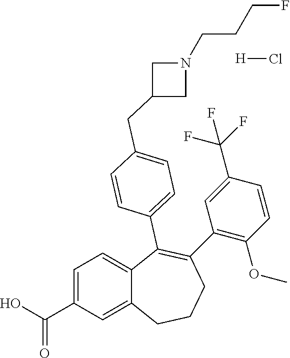 | 9-(4-((1-(3- fluoropropyl)azetidin-3- yl)methyl)phenyl)-8-(2- methoxy-5- (trifluoromethyl)phenyl)- 6,7-dihydro-5H- benzo[7]annulene-3- carboxylic acid, hydrochloride |
| 29 | 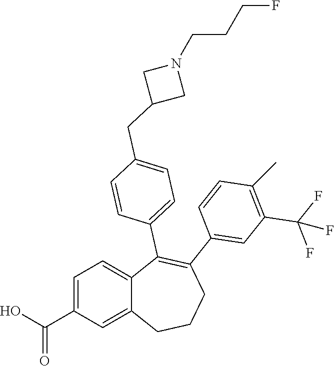 [SMILES: Cc1ccc(C2=C(c3ccc(CC4CN(CCCF)C4)cc3)c3ccc(C(=O)O)cc3CCC2)cc1C(F)(F)F] | 9-(4-((1-(3- fluoropropyl)azetidin-3- yl)methyl)phenyl)-8-(4- methyl-3- (trifluoromethyl)phenyl)- 6,7-dihydro-5H- benzo[7]annulene-3- carboxylic acid |
| 30 | 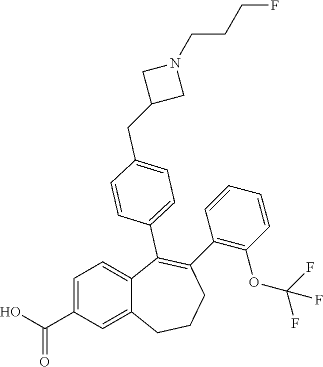 [SMILES: O=C(O)c1ccc2c(c1)CCCC(c1ccccc1OC(F)(F)F)=C2c1ccc(CC2CN(CCCF)C2)cc1] | 9-(4-((1-(3- fluoropropyl)azetidin-3- yl)methyl)phenyl)-8-(2- (trifluoromethoxy)phenyl)- 6,7-dihydro-5H- benzo[7]annulene-3- carboxylic acid |
| 31 | 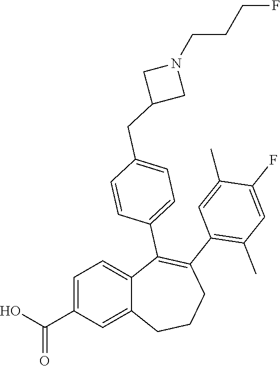 [SMILES: Cc1cc(C2=C(c3ccc(CC4CN(CCCF)C4)cc3)c3ccc(C(=O)O)cc3CCC2)c(C)cc1F] | 8-(4-fluoro-2,5- dimethylphenyl)-9-(4- ((1-(3- fluoropropyl)azetidin-3- yl)methyl)phenyl)-6,7- dihydro-5H- benzo[7]annulene-3- carboxylic acid |
| 32 | 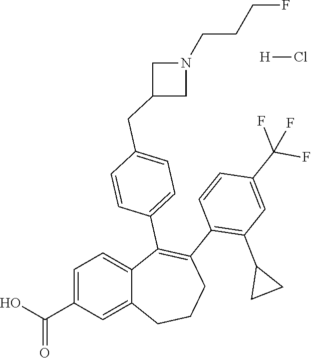 | 8-(2-cyclopropyl-4- (trifluoromethyl)phenyl)- 9-(4-((1-(3- fluoropropyl)azetidin-3- yl)methyl)phenyl)-6,7- dihydro-5H- benzo[7]annulene-3- carboxylic acid, hydrochloride |
| 33 | 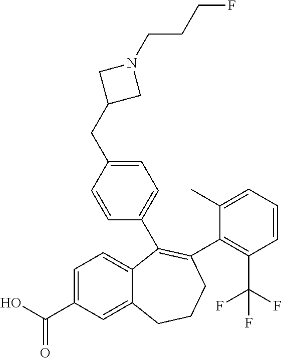 [SMILES: Cc1cccc(C(F)(F)F)c1C1=C(c2ccc(CC3CN(CCCF)C3)cc2)c2ccc(C(=O)O)cc2CCC1] | 9-(4-((1-(3- fluoropropyl)azetidin-3- yl)methyl)phenyl)-8-(2- methyl-6- (trifluoromethyl)phenyl)- 6,7-dihydro-5H- benzo[7]annulene-3- carboxylic acid |
| 34 | 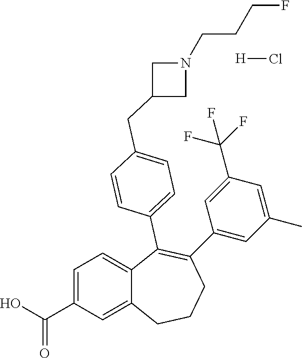 | 9-(4-((1-(3- fluoropropyl)azetidin-3- yl)methyl)phenyl)-8-(3- methyl-5- (trifluoromethyl)phenyl)- 6,7-dihydro-5H- benzo[7]annulene-3- carboxylic acid, hydrochloride |
| 35 | 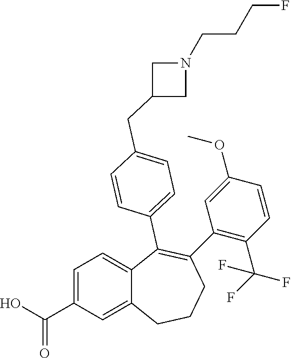 [SMILES: COc1ccc(C(F)(F)F)c(C2=C(c3ccc(CC4CN(CCCF)C4)cc3)c3ccc(C(=O)O)cc3CCC2)c1] | 9-(4-((1-(3- fluoropropyl)azetidin-3- yl)methyl)phenyl)-8-(5- methoxy-2- (trifluoromethyl)phenyl)- 6,7-dihydro-5H- benzo[7]annulene-3- carboxylic acid |
| 36 | 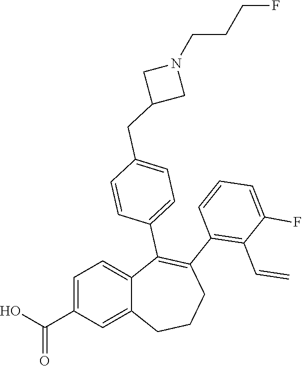 [SMILES: C=Cc1c(F)cccc1C1=C(c2ccc(CC3CN(CCCF)C3)cc2)c2ccc(C(=O)O)cc2CCC1] | 8-(3-fluoro-2- vinylphenyl)-9-(4-((1- (3- fluoropropyl)azetidin-3- yl)methyl)phenyl)-6,7- dihydro-5H- benzo[7]annulene-3- carboxylic acid |
| 37 | 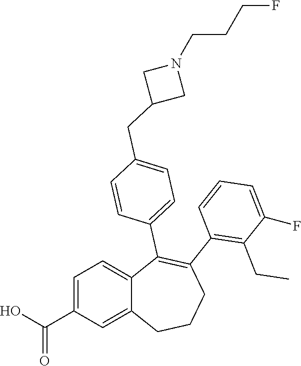 [SMILES: CCc1c(F)cccc1C1=C(c2ccc(CC3CN(CCCF)C3)cc2)c2ccc(C(=O)O)cc2CCC1] | 8-(2-ethyl-3- fluorophenyl)-9-(4-((1- (3- fluoropropyl)azetidin-3- yl)methyl)phenyl)-6,7- dihydro-5H- benzo[7]annulene-3- carboxylic acid |
| 38 | 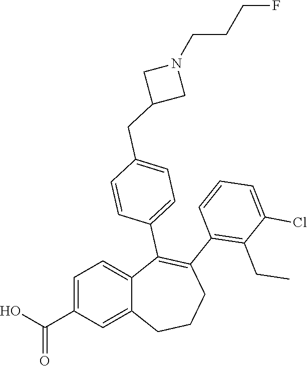 [SMILES: CCc1c(Cl)cccc1C1=C(c2ccc(CC3CN(CCCF)C3)cc2)c2ccc(C(=O)O)cc2CCC1] | 8-(3-chloro-2- ethylphenyl)-9-(4-((1- (3- fluoropropyl)azetidin-3- yl)methyl)phenyl)-6,7- dihydro-5H- benzo[7]annulene-3- carboxylic acid |
| 39 | 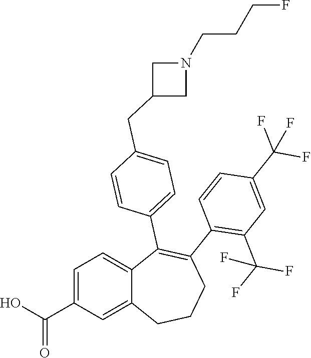 [SMILES: O=C(O)c1ccc2c(c1)CCCC(c1ccc(C(F)(F)F)cc1C(F)(F)F)=C2c1ccc(CC2CN(CCCF)C2)cc1] | 8-(2,4- bis(trifluoromethyl) phenyl)-9-(4-((1-(3- fluoropropyl)azetidin-3- yl)methyl)phenyl)-6,7- dihydro-5H- benzo[7]annulene-3- carboxylic acid |
| 40 | 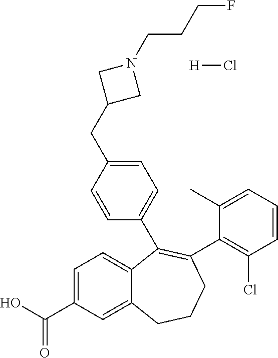 | 8-(2-chloro-6- methylphenyl)-9-(4-((1- (3- fluoropropyl)azetidin-3- yl)methyl)phenyl)-6,7- dihydro-5H- benzo[7]annulene-3- carboxylic acid, hydrochloride |
| 41 | 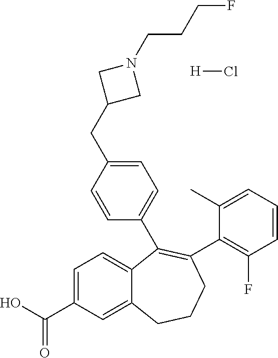 | 8-(2-fluoro-6- methylphenyl)-9-(4-((1- (3- fluoropropyl)azetidin-3- yl)methyl)phenyl)-6,7- dihydro-5H- benzo[7]annulene-3- carboxylic acid, hydrochloride |
| 42 | 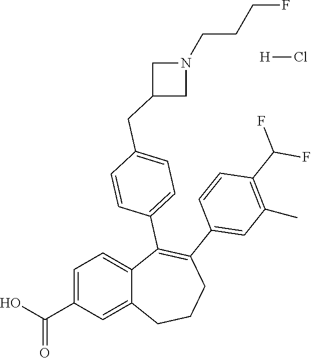 | 8-(4-(difluoromethyl)-3- methylphenyl)-9-(4-((1- (3- fluoropropyl)azetidin-3- yl)methyl)phenyl)-6,7- dihydro-5H- benzo[7]annulene-3- carboxylic acid, hydrochloride |
| 43 | 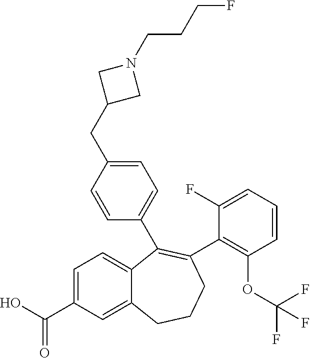 [SMILES: O=C(O)c1ccc2c(c1)CCCC(c1c(F)cccc1OC(F)(F)F)=C2c1ccc(CC2CN(CCCF)C2)cc1] | 8-(2-fluoro-6- (trifluoromethoxy) phenyl)-9-(4-((1-(3- fluoropropyl)azetidin-3- yl)methyl)phenyl)-6,7- dihydro-5H- benzo[7]annulene-3- carboxylic acid |
| 44 | 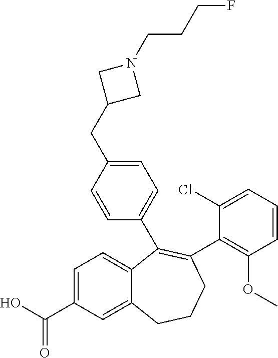 [SMILES: COc1cccc(Cl)c1C1=C(c2ccc(CC3CN(CCCF)C3)cc2)c2ccc(C(=O)O)cc2CCC1] | 8-(2-chloro-6- methoxyphenyl)-9-(4- ((1-(3- fluoropropyl)azetidin-3- yl)methyl)phenyl)-6,7- dihydro-5H- benzo[7]annulene-3- carboxylic acid |
| 45 | 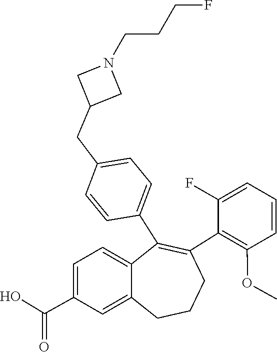 [SMILES: COc1cccc(F)c1C1=C(c2ccc(CC3CN(CCCF)C3)cc2)c2ccc(C(=O)O)cc2CCC1] | 8-(2-fluoro-6- methoxyphenyl)-9-(4- ((1-(3- fluoropropyl)azetidin-3- yl)methyl)phenyl)-6,7- dihydro-5H- benzo[7]annulene-3- carboxylic acid |
| 46 | 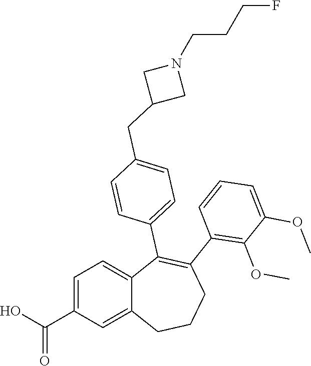 [SMILES: COc1cccc(C2=C(c3ccc(CC4CN(CCCF)C4)cc3)c3ccc(C(=O)O)cc3CCC2)c1OC] | 8-(2,3- dimethoxyphenyl)-9-(4- ((1-(3- fluoropropyl)azetidin-3- yl)methyl)phenyl)-6,7- dihydro-5H- benzo[7]annulene-3- carboxylic acid |
| 47 | 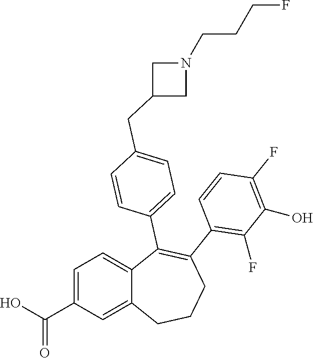 [SMILES: O=C(O)c1ccc2c(c1)CCCC(c1ccc(F)c(O)c1F)=C2c1ccc(CC2CN(CCCF)C2)cc1] | 8-(2,4-difluoro-3- hydroxyphenyl)-9-(4- ((1-(3- fluoropropyl)azetidin-3- yl)methyl)phenyl)-6,7- dihydro-5H- benzo[7]annulene-3- carboxylic acid |
| 48 | 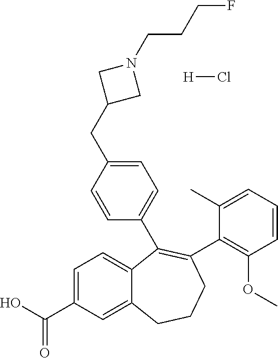 | 9-(4-((1-(3- fluoropropyl)azetidin-3- yl)methyl)phenyl)-8-(2- methoxy-6- methylphenyl)-6,7- dihydro-5H- benzo[7]annulene-3- carboxylic acid, hydrochloride |
| 49 | 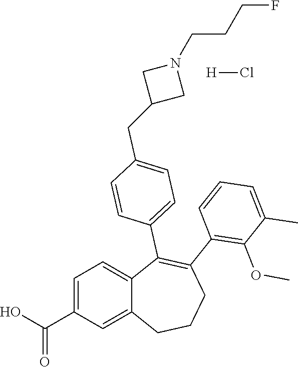 | 9-(4-((1-(3- fluoropropyl)azetidin-3- yl)methyl)phenyl)-8-(2- methoxy-3- methylphenyl)-6,7- dihydro-5H- benzo[7]annulene-3- carboxylic acid, hydrochloride |
| 50 | 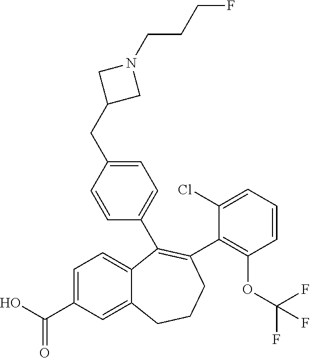 [SMILES: O=C(O)c1ccc2c(c1)CCCC(c1c(Cl)cccc1OC(F)(F)F)=C2c1ccc(CC2CN(CCCF)C2)cc1] | 8-(2-chloro-6- (trifluoromethoxy) phenyl)-9-(4-((1-(3- fluoropropyl)azetidin-3- yl)methyl)phenyl)-6,7- dihydro-5H- benzo[7]annulene-3- carboxylic acid |
| 51 | 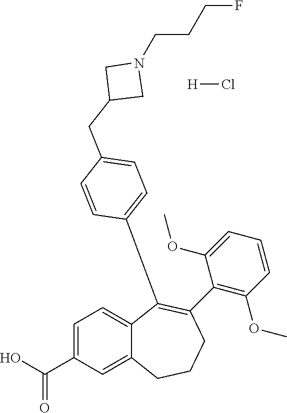 [SMILES: COc1cccc(OC)c1C1=C(c2ccc(CC3CN(CCCF)C3)cc2)c2ccc(C(=O)O)cc2CCC1 ; Cl] | 8-(2,6- dimethoxyphenyl)-9-(4- ((1-(3- fluoropropyl)azetidin-3- yl)methyl)phenyl)-6,7- dihydro-5H- benzo[7]annulene-3- carboxylic acid, hydrochloride |
| 52 | 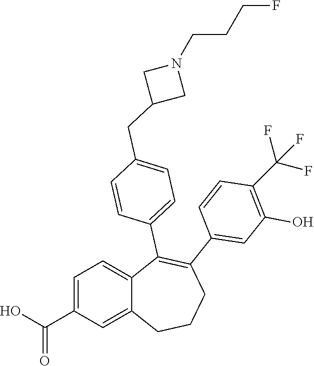 [SMILES: O=C(O)c1ccc2c(c1)CCCC(c1ccc(C(F)(F)F)c(O)c1)=C2c1ccc(CC2CN(CCCF)C2)cc1] | 9-(4-((1-(3- fluoropropyl)azetidin-3- yl)methyl)phenyl)-8-(3- hydroxy-4- (trifluoromethyl)phenyl)- 6,7-dihydro-5H- benzo[7]annulene-3- carboxylic acid |
| 53 | 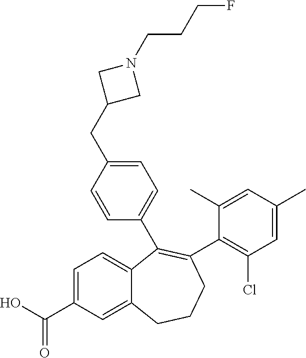 [SMILES: Cc1cc(C)c(C2=C(c3ccc(CC4CN(CCCF)C4)cc3)c3ccc(C(=O)O)cc3CCC2)c(Cl)c1] | 8-(2-chloro-4,6- dimethylphenyl)-9-(4- ((1-(3- fluoropropyl)azetidin-3- yl)methyl)phenyl)-6,7- dihydro-5H- benzo[7]annulene-3- carboxylic acid |
| 54 | 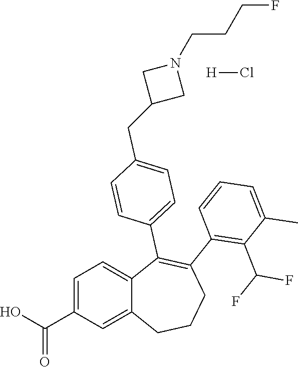 | 8-(2-(difluoromethyl)-3- methylphenyl)-9-(4-((1- (3- fluoropropyl)azetidin-3- yl)methyl)phenyl)-6,7- dihydro-5H- benzo[7]annulene-3- carboxylic acid, hydrochloride |
| 55 | 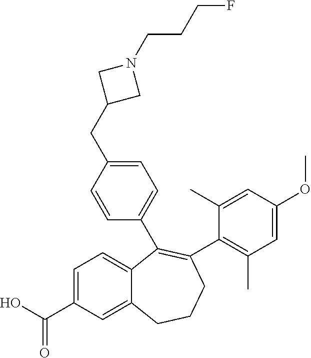 [SMILES: COc1cc(C)c(C2=C(c3ccc(CC4CN(CCCF)C4)cc3)c3ccc(C(=O)O)cc3CCC2)c(C)c1] | 9-(4-((1-(3- fluoropropyl)azetidin-3- yl)methyl)phenyl)-8-(4- methoxy-2,6- dimethylphenyl)-6,7- dihydro-5H- benzo[7]annulene-3- carboxylic acid |
| 56 | 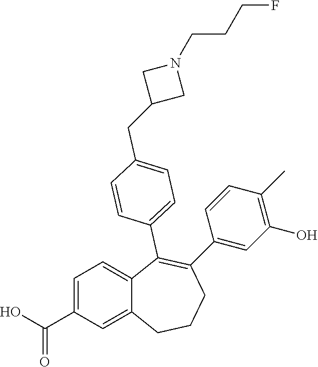 [SMILES: Cc1ccc(C2=C(c3ccc(CC4CN(CCCF)C4)cc3)c3ccc(C(=O)O)cc3CCC2)cc1O] | 9-(4-((1-(3- fluoropropyl)azetidin-3- yl)methyl)phenyl)-8-(3- hydroxy-4- methylphenyl)-6,7- dihydro-5H- benzo[7]annulene-3- carboxylic acid |
| 57 | 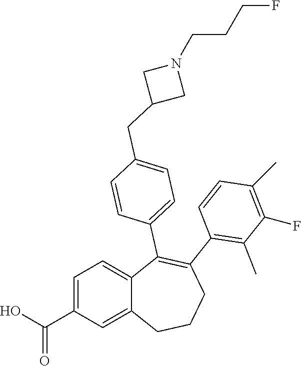 [SMILES: Cc1ccc(C2=C(c3ccc(CC4CN(CCCF)C4)cc3)c3ccc(C(=O)O)cc3CCC2)c(C)c1F] | 8-(3-fluoro-2,4- dimethylphenyl)-9-(4- ((1-(3- fluoropropyl)azetidin-3- yl)methyl)phenyl)-6,7- dihydro-5H- benzo[7]annulene-3- carboxylic acid |
| 58 | 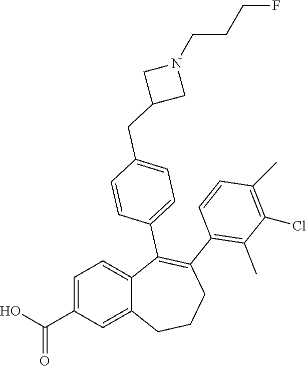 [SMILES: Cc1ccc(C2=C(c3ccc(CC4CN(CCCF)C4)cc3)c3ccc(C(=O)O)cc3CCC2)c(C)c1Cl] | 8-(3-chloro-2,4- dimethylphenyl)-9-(4- ((1-(3- fluoropropyl)azetidin-3- yl)methyl)phenyl)-6,7- dihydro-5H- benzo[7]annulene-3- carboxylic acid |
| 59 | 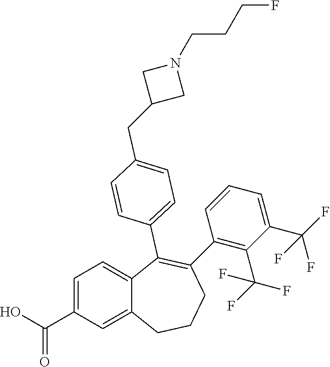 [SMILES: O=C(O)c1ccc2c(c1)CCCC(c1cccc(C(F)(F)F)c1C(F)(F)F)=C2c1ccc(CC2CN(CCCF)C2)cc1] | 8-(2,3- bis(trifluoromethyl) phenyl)-9-(4-((1-(3- fluoropropyl)azetidin-3- yl)methyl)phenyl)-6,7- dihydro-5H- benzo[7]annulene-3- carboxylic acid |
| 60 | 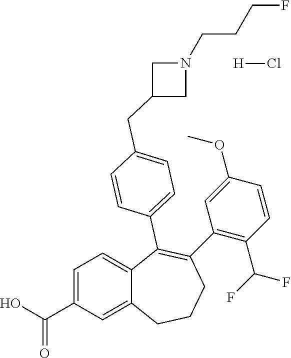 | 8-(2-(difluoromethyl)-5- methoxyphenyl)-9-(4- ((1-(3- fluoropropyl)azetidin-3- yl)methyl)phenyl)-6,7- dihydro-5H- benzo[7]annulene-3- carboxylic acid, hydrochloride |
| 61 | 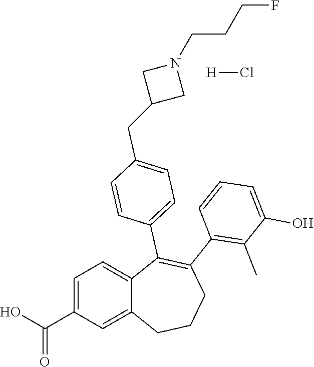 | 9-(4-((1-(3- fluoropropyl)azetidin-3- yl)methyl)phenyl)-8-(3- hydroxy-2- methylphenyl)-6,7- dihydro-5H- benzo[7]annulene-3- carboxylic acid, hydrochloride |
| 62 | 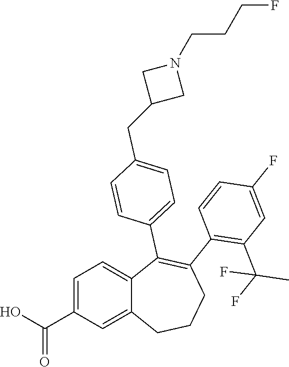 [SMILES: CC(F)(F)c1cc(F)ccc1C1=C(c2ccc(CC3CN(CCCF)C3)cc2)c2ccc(C(=O)O)cc2CCC1] | 8-(2-(1,1-difluoroethyl)- 4-fluorophenyl)-9-(4- ((1-(3- fluoropropyl)azetidin-3- yl)methyl)phenyl)-6,7- dihydro-5H- benzo[7]annulene-3- carboxylic acid |
| 63 | 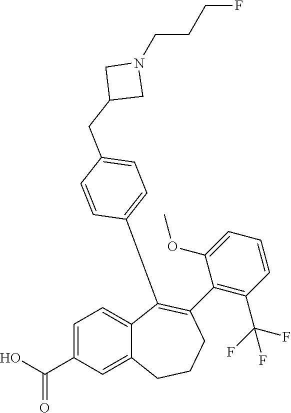 [SMILES: COc1cccc(C(F)(F)F)c1C1=C(c2ccc(CC3CN(CCCF)C3)cc2)c2ccc(C(=O)O)cc2CCC1] | 9-(4-((1-(3- fluoropropyl)azetidin-3- yl)methyl)phenyl)-8-(2- methoxy-6- (trifluoromethyl)phenyl)- 6,7-dihydro-5H- benzo[7]annulene-3- carboxylic acid |
| 64 | 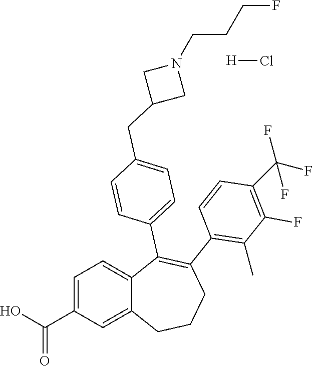 | 8-(3-fluoro-2-methyl-4- (trifluoromethyl)phenyl)- 9-(4-((1-(3- fluoropropyl)azetidin-3- yl)methyl)phenyl)-6,7- dihydro-5H- benzo[7]annulene-3- carboxylic acid, hydrochloride |
| 65 | 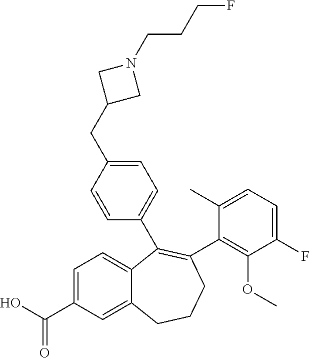 [SMILES: COc1c(F)ccc(C)c1C1=C(c2ccc(CC3CN(CCCF)C3)cc2)c2ccc(C(=O)O)cc2CCC1] | 8-(3-fluoro-2-methoxy- 6-methylphenyl)-9-(4- ((1-(3- fluoropropyl)azetidin-3- yl)methyl)phenyl)-6,7- dihydro-5H- benzo[7]annulene-3- carboxylic acid |
| 66 | 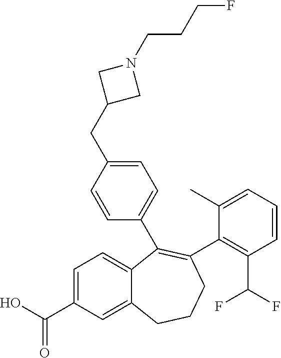 [SMILES: Cc1cccc(C(F)F)c1C1=C(c2ccc(CC3CN(CCCF)C3)cc2)c2ccc(C(=O)O)cc2CCC1] | 8-(2-(difluoromethyl)-6- methylphenyl)-9-(4-((1- (3- fluoropropyl)azetidin-3- yl)methyl)phenyl)-6,7- dihydro-5H- benzo[7]annulene-3- carboxylic acid |
| 67 | 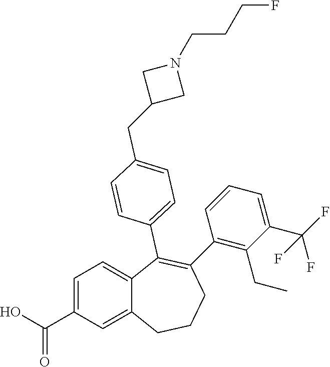 [SMILES: CCc1c(C2=C(c3ccc(CC4CN(CCCF)C4)cc3)c3ccc(C(=O)O)cc3CCC2)cccc1C(F)(F)F] | 8-(2-ethyl-3- (trifluoromethyl)phenyl)- 9-(4-((1-(3- fluoropropyl)azetidin-3- yl)methyl)phenyl)-6,7- dihydro-5H- benzo[7]annulene-3- carboxylic acid |
| 68 | 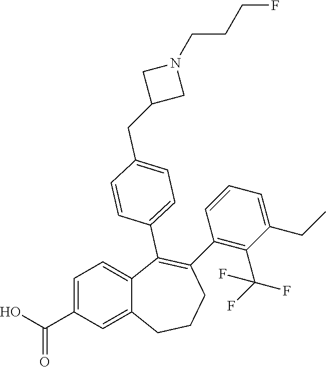 [SMILES: CCc1cccc(C2=C(c3ccc(CC4CN(CCCF)C4)cc3)c3ccc(C(=O)O)cc3CCC2)c1C(F)(F)F] | 8-(3-ethyl-2- (trifluoromethyl)phenyl)- 9-(4-((1-(3- fluoropropyl)azetidin-3- yl)methyl)phenyl)-6,7- dihydro-5H- benzo[7]annulene-3- carboxylic acid |
| 69 | 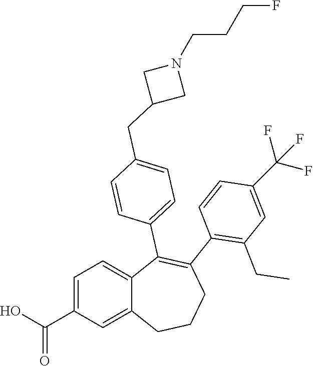 [SMILES: CCc1cc(C(F)(F)F)ccc1C1=C(c2ccc(CC3CN(CCCF)C3)cc2)c2ccc(C(=O)O)cc2CCC1] | 8-(2-ethyl-4- (trifluoromethyl)phenyl)- 9-(4-((1-(3- fluoropropyl)azetidin-3- yl)methyl)phenyl)-6,7- dihydro-5H- benzo[7]annulene-3- carboxylic acid |
| 70 | 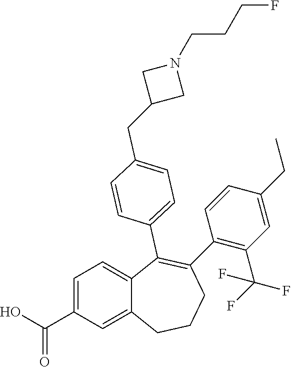 [SMILES: CCc1ccc(C2=C(c3ccc(CC4CN(CCCF)C4)cc3)c3ccc(C(=O)O)cc3CCC2)c(C(F)(F)F)c1] | 8-(4-ethyl-2- (trifluoromethyl)phenyl)- 9-(4-((1-(3- fluoropropyl)azetidin-3- yl)methyl)phenyl)-6,7- dihydro-5H- benzo[7]annulene-3- carboxylic acid |
| 71 | 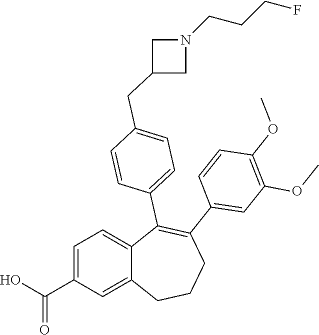 [SMILES: COc1ccc(C2=C(c3ccc(CC4CN(CCCF)C4)cc3)c3ccc(C(=O)O)cc3CCC2)cc1OC] | 8-(3,4- dimethoxyphenyl)-9-(4- ((1-(3- fluoropropyl)azetidin-3- yl)methyl)phenyl)-6,7- dihydro-5H- benzo[7]annulene-3- carboxylic acid |
| 72 | 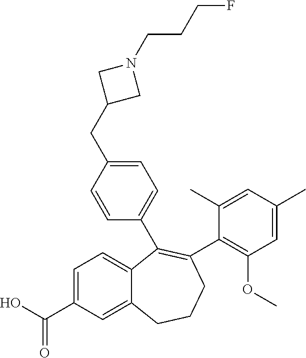 [SMILES: COc1cc(C)cc(C)c1C1=C(c2ccc(CC3CN(CCCF)C3)cc2)c2ccc(C(=O)O)cc2CCC1] | 9-(4-((1-(3- fluoropropyl)azetidin-3- yl)methyl)phenyl)-8-(2- methoxy-4,6- dimethylphenyl)-6,7- dihydro-5H- benzo[7]annulene-3- carboxylic acid |
| 73 | 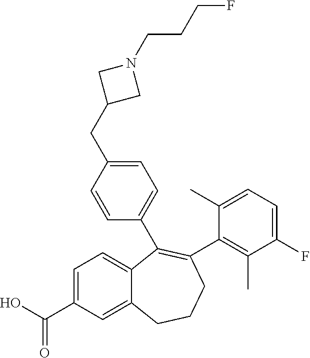 [SMILES: Cc1ccc(F)c(C)c1C1=C(c2ccc(CC3CN(CCCF)C3)cc2)c2ccc(C(=O)O)cc2CCC1] | 8-(3-fluoro-2,6- dimethylphenyl)-9-(4- ((1-(3- fluoropropyl)azetidin-3- yl)methyl)phenyl)-6,7- dihydro-5H- benzo[7]annulene-3- carboxylic acid |

TABLE 1b

| | | | MASS: |
|---|---|---|---|
| | | | LC/MS |
| | Preparation | | (m/z, |
| Ex | Method | NMR | MH+) |
| 1 | A | 1H NMR (400 MHz, DMSO-d6) δ ppm 1.51-1.69 (m, 2 H), | 554 |
| | | 2.07-2.28 (m, 4 H), 2.40 (t, J = 7 Hz, 2 H), 2.52-2.59 (m | |
| | | partially hidden, 1 H), 2.64-2.76 (m, 4 H), 2.89 (t, J = 6 Hz, | |
| | | 2 H), 3.23 (br t, J = 7 Hz, 2 H), 4.26-4.56 (m, 2 H), 6.75 (d, | |
| | | J = 8 Hz, 2 H), 6.83 (d, J = 8 Hz, 1 H), 6.92 (d, J = 8 Hz, 2 H), | |
| | | 6.93-7.27 (m, 4 H), 7.73 (dd, J = 8, 2 Hz, 1 H), 7.90 (d, J = 2 | |
| | | Hz, 1 H) | |
| 2 | A | 1H NMR (400 MHz, DMSO-d6) δ ppm 1.40-1.79 (m, 6 H), | 524 |
| | | 2.10-2.15 (m, 4 H), 2.39 (t, J = 7 Hz, 2 H), 2.53-2.77 (m, 9 | |
| | | H), 2.80-2.96 (m, 2 H), 3.21 (br t, J = 7 Hz, 2 H), 4.41 (dt, | |
| | | J = 47, 6 Hz, 2 H), 6.67 (d, J = 8 Hz, 2 H), 6.77-7.01 (m, 6 H), | |
| | | 7.73 (dd, J = 8, 2 Hz, 1 H), 7.89 (d, J = 2 Hz, 1 H), 11.06-.01 | |
| | | (m, 1 H) | |
| 3 | E | 1H NMR (400 MHz, DMSO-d6) δ ppm 1.16-1.78 (m, 8 | 492 |
| | | H), 1.80-1.99 (m, 3 H), 2.03-2.21 (m, 2 H), 2.43 (t, J = 7 | |
| | | Hz, 2 H), 2.58-2.87 (m, 8 H), 3.14-3.46 (m partially | |
| | | hidden, 5 H), 4.43 (dt, J = 47, 6 Hz, 2 H), 6.67 (d, J = 8 Hz, 1 | |
| | | H), 6.95 (d, J = 8 Hz, 2 H), 7.12 (d, J = 8 Hz, 2 H), 7.62 (dd, | |
| | | J = 8, 2 Hz, 1 H), 7.80 (d, J = 2 Hz, 1 H), 11.30-14.17 (m, 1 | |
| | | H) | |
| 4 | A | 1H NMR (400 MHz, DMSO-d6) δ ppm 1.54-1.67 (m, 2 | 568 |
| | | H), 2.06-2.25 (m, 4 H), 2.41 (t, J = 7 Hz, 2 H), 2.67-2.80 | |
| | | (m, 5 H), 2.81-2.99 (m, 2 H), 3.24 (br t, J = 7 Hz, 2 H), 3.90 | |
| | | (s, 3 H), 4.42 (dt, J = 47, 6 Hz, 2 H), 6.80-6.89 (m, 3 H), | |
| | | 6.95 (d, J = 8 Hz, 2 H), 7.02 (t, J = 8 Hz, 1 H), 7.20 (dd, J = 8, 2 | |
| | | Hz, 1 H), 7.47 (dd, J = 8, 2 Hz, 1 H), 7.74 (dd, J = 8, 2 Hz, 1 | |
| | | H), 7.91 (d, J = 2 Hz, 1 H) | |
| 5 | A | 1H NMR (400 MHz, DMSO-d6) δ ppm 1.53-1.66 (m, 2 | 588 |
| | | H), 2.14-2.26 (m, 4 H), 2.39 (t, J = 8 Hz, 2 H), 2.53-2.58 | |
| | | (m hidden, 1 H), 2.62-2.74 (m, 4 H), 2.95 (br t, J = 6 Hz, 2 | |
| | | H), 3.17-3.23 (m, 2 H), 4.41 (dt, J = 47, 6 Hz, 2 H), 6.74 (d, | |
| | | J-8 Hz, 2 H), 6.86 (d, J = 8 Hz, 1 H), 6.90 (d, J = 8 Hz, 2 H), | |
| | | 7.25 (m, 1 H), 7.30 (t, J = 8 Hz, 1 H), 7.36 (m, 1 H), 7.75 | |
| | | (dd, J = 8, 2 Hz, 1 H), 7.92 (d, J = 2 Hz, 1 H) | |
| 6 | A | 1H NMR (400 MHz, DMSO-d6) δ ppm 1.52-1.65 (m, 2 H), | 568 |
| | | 2.06 (s, 3 H), 2.12-2.25 (m, 4 H), 2.38 (t, J = 7 Hz, 2 H), | |
| | | 2.55 (m partially hidden, 1 H), 2.64-2.72 (m, 4 H), 2.79- | |
| | | 3.03 (m, 2 H), 3.16-3.22 (m, 2 H), 4.41 (dt, J = 47, 6 Hz, 2 | |
| | | H), 6.66 (d, J = 7 Hz, 2 H), 6.83-6.89 (m, 3 H), 7.10-7.22 | |
| | | (m, 3 H), 7.74 (dd, J = 8, 2 Hz, 1 H), 7.91 (d, J = 2 Hz, 1 H) | |
| 7 | A | 1H NMR (400 MHz, DMSO-d6) δ ppm 1.50-1.69 (m, 2 | 514 |
| | | H), 2.02 (s, 3 H), 2.06-2.22 (m, 4 H), 2.40 (t, J = 7 Hz, 2 H), | |
| | | 2.52-2.76 (m partially hidden, 5 H), 2.81-2.98 (m, 2 H), | |
| | | 3.20-3.25 (m, 2 H), 3.74 (s, 3 H), 4.41 (dt, J = 47, 6 Hz, 2 | |
| | | H), 6.61 (d, J = 7 Hz, 1 H), 6.70 (d, J = 8 Hz, 2 H), 6.76 (d, | |
| | | J = 8 Hz, 1 H), 6.83 (d, J = 8 Hz, 1 H), 6.86 (d, J = 8 Hz, 2 H), | |
| | | 6.99 (t, J = 8 Hz, 1 H), 7.73 (dd, J = 8, 2 Hz, 1 H), 7.89 (d, J = 2 | |
| | | Hz, 1 H) | |
| 8 | B | 1H NMR (400 MHz, DMSO-d6) δ ppm 1.54-1.67 (m, 2 | 550 |
| | | H), 2.07-2.24 (m, 4 H), 2.41 (t, J = 7 Hz, 2 H), 2.51-2.60 | |
| | | (m hidden, 2 H), 2.65-2.76 (m, 3 H), 2.77-2.85 (m, 1 H), | |
| | | 2.88-3.01 (m, 1 H), 3.24 (t, J = 7 Hz, 2 H), 3.81 (s, 3 H), | |
| | | 4.41 (dt, J = 47, 6 Hz, 2 H), 6.64 (d, J = 8 Hz, 1 H), 6.76 (d, | |
| | | J = 8 Hz, 2 H), 6.82 (d, J = 8 Hz, 1 H), 6.89 (d, J = 8 Hz, 2 H), | |
| | | 6.96 (d, J = 8 Hz, 1 H), 7.03 (t, J = 54 Hz, 1 H), 7.25 (t, J = 8 | |
| | | Hz, 1 H), 7.73 (dd, J = 8, 2 Hz, 1 H), 7.88 (d, J = 2 Hz, 1 H) | |
| 9 | A | 1H NMR (400 MHz, DMSO-d6) δ ppm 1.48-1.72 (m, 2 | 526 |
| | | H), 1.77-1.91 (m, 2 H), 2.01-2.21 (m, 4 H), 2.41 (t, J = 8 | |
| | | Hz, 2 H), 2.50-2.54 (m hidden, 2 H), 2.63-2.76 (m, 5 H), | |
| | | 2.85 (br t, J = 6 Hz, 2 H), 3.23 (t, J = 7 Hz, 2 H), 3.94-4.10 | |
| | | (m, 2 H), 4.41 (dt, J = 47, 6 Hz, 2 H), 6.58 (t, J = 8 Hz, 1 H), | |
| | | 6.68-6.77 (m, 3 H), 6.77-6.88 (m, 4 H), 7.71 (dd, J = 8, 2 | |
| | | Hz, 1 H), 7.87 (d, J = 2 Hz, 1 H) | |
| 10 | F | 1H NMR (400 MHz, DMSO-d6) δ ppm 0.80-1.65 (m, 8 | 523 |
| | | H), 0.82 (s, 3 H), 0.89 (s, 3 H), 1.69-1.90 (m, 2 H), 1.90- | |
| | | 2.04 (m, 4 H), 2.12-2.24 (m, 2 H), 2.54-2.61 (m, 1 H), | |
| | | 2.74 (t, J = 6 Hz, 2 H), 2.99 (br s, 3 H), 3.59 (br s, 2 H), 3.89 | |
| | | (br s, 2 H), 4.49 (dt, J = 48, 6 Hz, 2 H), 6.81 (d, J = 8 Hz, 1 | |
| | | H), 7.58 (dd, J = 10, 2 Hz, 1 H), 7.66 (dd, J = 8, 2 Hz, 1 H), | |
| | | 7.83 (d, J = 2 Hz, 1 H), 8.33 (s, 1 H), 10.36 (s, 1 H), 12.64 | |
| | | (br s, 1 H) | |
| 11 | B | 1H NMR (400 MHz, DMSO-d6) δ ppm 1.51-1.69 (m, 2 | 568 |
| | | H), 1.96-2.25 (m, 4 H), 2.39 (t, J = 8 Hz, 2 H), 2.52-2.75 | |
| | | (m partially hidden, 5 H), 2.81 (m, 1 H), 2.97 (m, 1 H), 3.22 | |
| | | (t, J = 8 Hz, 2 H), 3.86 (s, 3 H), 4.41 (dt, J = 47, 6 Hz, 2 H), | |
| | | 6.65 (d, J = 8 Hz, 1 H), 6.77 (d, J = 8 Hz, 2 H), 6.82 (d, J = 8 | |
| | | Hz, 1 H), 6.92 (d, J = 8 Hz, 2 H), 7.06 (d, J = 8 Hz, 1 H), 7.32 | |
| | | (t, J = 8 Hz, 1 H), 7.73 (dd, J = 8, 2 Hz, 1 H), 7.88 (d, J = 2 Hz, | |
| | | 1 H), 10.90-14.58 (m, 1 H) | |
| 12 | B | 1H NMR (400 MHz, DMSO-d6) δ ppm 1.51-1.74 (m, 3 | 526 |
| | | H), 1.87 (m, 1 H), 2.11-2.17 (m, 4 H), 2.40 (t, J = 7 Hz, 2 | |
| | | H), 2.52-2.69 (m, 5 H), 2.72 (t, J = 7 Hz, 2 H), 2.79-2.95 | |
| | | (m, 2 H), 3.23 (br t, J = 7 Hz, 2 H), 3.91-4.08 (m, 2 H), 4.41 | |
| | | (dt, J = 47, 6 Hz, 2 H), 6.52-6.60 (m, 2 H), 6.68 (d, J = 8 Hz, | |
| | | 2 H), 6.84 (d, J = 8 Hz, 1 H), 6.88 (d, J = 8 Hz, 2 H), 6.92 (m, | |
| | | 1 H), 7.73 (d, J = 8 Hz, 1 H), 7.89 (d, J = 2 Hz, 1 H), 11.28- | |
| | | 14.11 (m, 1 H) | |
| 13 | F | 1H NMR (400 MHz, DMSO-d6) δ ppm 0.82 (s, 3 H), 0.89 | 541 |
| | | (s, 3 H), 0.90-1.64 (m, 8 H), 1.76 (m, 1 H), 1.91-2.04 (m, | |
| | | 4 H), 2.09-2.24 (m, 2 H), 2.40-2.47 (m, 2 H), 2.59-2.67 | |
| | | (m hidden, 1 H), 2.74 (br t, J = 7 Hz, 2 H), 2.80 (t, J = 6 Hz, 2 | |
| | | H), 2.88 (br d, J = 8 Hz, 2 H), 3.23-3.26 (m hidden, 2 H), | |
| | | 6.04 (tt, J = 57, 6 Hz, 1 H), 6.81 (d, J = 8 Hz, 1 H), 7.54 (br d, | |
| | | J = 9 Hz, 1 H), 7.66 (d, J = 8 Hz, 1 H), 7.83 (s, 1 H), 8.30 (s, 1 | |
| | | H) | |
| 14 | B | 1H NMR (400 MHz, DMSO-d6) δ ppm 1.49-1.70 (m, 2 | 550 |
| | | H), 2.07 (s, 3 H), 2.09-2.23 (m, 4 H), 2.40 (t, J = 7 Hz, 2 H), | |
| | | 2.52 (m hidden, 2 H), 2.64-2.75 (m, 3 H), 2.81-3.00 (m, 2 | |
| | | H), 3.22 (br t, J = 7 Hz, 2 H), 4.41 (dt, J = 47, 6 Hz, 2 H), 6.68 | |
| | | (d, J = 8 Hz, 2 H), 6.85 (d, J = 8 Hz, 1 H), 6.88 (d, J = 8 Hz, 2 | |
| | | H), 6.90-7.31 (m, 4 H), 7.74 (dd, J = 8, 2 Hz, 1 H), 7.90 (d, | |
| | | J = 2 Hz, 1 H) | |
| 15 | C | 1H NMR (400 MHz, DMSO-d6) δ ppm 1.50-1.74 (m, 2 | 452 |
| | | H), 1.77-1.89 (m, 2 H), 1.91 (t, J = 7 Hz, 2 H), 2.09-2.25 | |
| | | (m, 4 H), 2.73 (t, J = 7 Hz, 2 H), 2.86-3.13 (m, 3 H), 3.21- | |
| | | 3.35 (m partially hidden, 4 H), 3.77-4.40 (m, 5 H), 4.51 | |
| | | (dt, J = 48, 6 Hz, 2 H), 6.74 (d, J = 8 Hz, 1 H), 7.02 (m, 2 H), | |
| | | 7.17 (m, 2 H), 7.66 (dd, J = 8, 2 Hz, 1 H), 7.83 (d, J = 2 Hz, 1 | |
| | | H), 9.70 (br s, 1 H), 12.76 (br s, 1 H) | |
| 16 | A | 1H NMR (400 MHz, DMSO-d6) δ ppm 1.76-1.94 (m, 2 | 568 |
| | | H), 2.14 (m, 4 H), 2.53-2.63 (m hidden, 2 H), 2.74-3.00 | |
| | | (m, 3 H), 3.09-3.25 (m, 2 H), 3.68-3.77 (m, 2 H), 3.81 (s, | |
| | | 3 H), 3.89-4.07 (m, 2 H), 4.50 (dt, J = 47, 6 Hz, 2 H), 6.77 | |
| | | (d, J = 8 Hz, 2 H), 6.84 (d, J = 8 Hz, 1 H), 6.94 (d, J = 8 Hz, 2 | |
| | | H), 7.07-7.16 (m, 2 H), 7.23 (d, J = 1 Hz, 1 H), 7.74 (dd, | |
| | | J = 8, 2 Hz, 1 H), 7.91 (d, J = 2 Hz, 1 H), 10.32 (br s, 1 H), | |
| | | 12.84 (br s, 1 H) | |
| 17 | A | 1H NMR (400 MHz, DMSO-d6) δ ppm 1.78-1.94 (m, 4 | 568 |
| | | H), 2.08-2.22 (m, 2 H), 2.30 (t, J = 6 Hz, 2 H), 2.79-2.94 | |
| | | (m, 3 H), 3.14-3.26 (m, 2 H), 3.71-3.81 (m, 2 H), 3.84 (s, | |
| | | 3 H), 3.93-4.01 (m, 2 H), 4.51 (dt, J = 47, 6 Hz, 2 H), 6.78- | |
| | | 6.86 (m, 3 H), 7.01 (d, J = 8 Hz, 2 H), 7.12 (d, J = 9 Hz, 1 H), | |
| | | 7.21 (d, J = 2 Hz, 1 H), 7.50 (dd, J = 9, 2 Hz, 1 H), 7.73 (dd, | |
| | | J = 8, 2 Hz, 1 H), 7.90 (d, J = 2 Hz, 1 H), 10.38 (br s, 1 H), | |
| | | 12.79 (br s, 1 H) | |
| 18 | A | 1H NMR (400 MHz, DMSO-d6) δ ppm 1.59-1.79 (m, 2 | 552 |
| | | H), 2.10-2.24 (m, 4 H), 2.25 (s, 3 H), 2.62-2.74 (m, 5 H), | |
| | | 2.94 (br dd, J = 21, 6 Hz, 2 H), 3.10 (br s, 2 H), 3.42-3.59 | |
| | | (m, 2 H), 4.44 (dt, J = 47, 6 Hz, 2 H), 6.70 (d, J = 8 Hz, 2 H), | |
| | | 6.86-6.92 (m, 3 H), 7.27 (d, J = 2 Hz, 1 H), 7.35 (d, J = 8 Hz, | |
| | | 1 H), 7.42 (dd, J = 8, 2 Hz, 1 H), 7.75 (dd, J = 8, 2 Hz, 1 H), | |
| | | 7.93 (d, J = 2 Hz, 1 H), 12.21 (s, 1 H) | |
| 19 | A | 1H NMR (400 MHz, DMSO-d6) δ ppm 1.52-1.70 (m, 2 | 568 |
| | | H), 2.05-2.27 (m, 2 H), 2.29-2.37 (m, 2 H), 2.40 (t, J = 7 | |
| | | Hz, 2 H), 2.57 (m, 1 H), 2.68-2.74 (m, 4 H), 2.87 (t, J = 7 | |
| | | Hz, 2 H), 3.22 (t, J = 7 Hz, 2 H), 3.62 (s, 3 H), 4.43 (dt, J = 47, | |
| | | 6 Hz, 2 H), 6.78-6.85 (m, 3 H), 6.88 (d, J = 9 Hz, 1 H), 6.91- | |
| | | 7.00 (m, 3 H), 7.40 (d, J = 8 Hz, 1 H), 7.72 (dd, J = 8, 2 Hz, | |
| | | 1 H), 7.89 (d, J = 2 Hz, 1 H), 13.08 (s, 1 H) | |
| 20 | A | 1H NMR (400 MHz, DMSO-d6) δ ppm 1.62-1.79 (m, 2 | 552 |
| | | H), 2.12-2.19 (m, 4 H), 2.20 (s, 3 H), 2.62-2.79 (m, 5 H), | |
| | | 2.87 (m, 1 H), 2.99 (m, 1 H), 3.07-3.17 (m, 2 H), 3.46- | |
| | | 3.58 (m, 2 H), 4.45 (dt, J = 48, 6 Hz, 2 H), 6.76 (d, J = 8 Hz, 2 | |
| | | H), 6.85 (d, J = 8 Hz, 1 H), 6.91 (d, J = 8 Hz, 2 H), 7.00 (s, 1 | |
| | | H), 7.21 (d, J = 8 Hz, 1 H), 7.57 (d, J = 8 Hz, 1 H), 7.74 (dd, | |
| | | J = 8, 2 Hz, 1 H), 7.91 (d, J = 2 Hz, 1 H), 12.22 (br s, 1 H) | |
| 21 | D | 1H NMR (400 MHz, DMSO-d6) δ ppm 1.60-2.06 (m, 8 | 478 |
| | | H), 2.07-2.23 (m, 3 H), 2.65-2.76 (m, 2 H), 2.85-3.09 | |
| | | (m, 3 H), 3.11-3.23 (m, 3 H), 3.31-3.35 (m hidden, 2 H), | |
| | | 3.70-3.88 (m, 2 H), 3.92-4.18 (m, 2 H), 4.41 (m, 1 H), | |
| | | 4.50 (dt, J = 47, 6 Hz, 2 H), 6.68 (d, J = 8 Hz, 1 H), 6.94 (d, | |
| | | J = 8 Hz, 2 H), 7.17 (d, J = 8 Hz, 2 H), 7.65 (dd, J = 8, 2 Hz, 1 | |
| | | H), 7.83 (d, J = 2 Hz, 1 H), 10.46 (br s, 1 H), 12.75 (br s, 1 | |
| | | H) | |
| 22 | A | 1H NMR (400 MHz, DMSO-d6) δ ppm 1.79-1.93 (m, 2 | 554 |
| | | H), 2.11-2.21 (m, 2 H), 2.26-2.33 (m, 2 H), 2.87 (br t, | |
| | | J = 7 Hz, 4 H), 2.86-2.99 (m, 1 H), 3.20 (br t, J = 7 Hz, 2 H), | |
| | | 3.67-3.82 (m, 2 H), 3.93-4.06 (m, 2 H), 4.50 (dt, J = 47, 6 | |
| | | Hz, 2 H), 6.78-6.82 (m, 2 H), 6.84 (d, J = 8 Hz, 1 H), 6.95- | |
| | | 7.02 (m, 3 H), 7.08-7.16 (m, 1 H), 7.26 (dt, J = 8, 1 Hz, 1 | |
| | | H), 7.37 (t, J = 9 Hz, 1 H), 7.74 (dd, J = 8, 2 Hz, 1 H), 7.91 (d, | |
| | | J = 2 Hz, 1 H), 10.38 (br s, 1 H), 12.83 (br s, 1 H) | |
| 23 | A | 1H NMR (400 MHz, DMSO-d6) δ ppm 1.65-1.87 (m, 2 | 534 |
| | | H), 2.09-2.24 (m, 4 H), 2.25 (s, 3 H), 2.70-3.02 (m, 7 H), | |
| | | 3.21-3.30 (m hidden, 2 H), 3.58-3.78 (m, 2 H), 4.47 (dt, | |
| | | J = 47, 6 Hz, 2 H), 6.85 (t, J = 55 Hz, 1 H), 6.77 (d, J = 8 Hz, 2 | |
| | | H), 6.85 (d, J = 8 Hz, 1 H), 6.92 (d, J = 8 Hz, 2 H), 7.05 (s, 1 | |
| | | H), 7.17 (d, J = 9 Hz, 1 H), 7.38 (d, J = 8 Hz, 1 H), 7.75 (dd, | |
| | | J = 8, 2 Hz, 1 H), 7.91 (d, J = 2 Hz, 1 H), 11.95 (br s, 1 H) | |
| 24 | B | 1H NMR (400 MHz, DMSO-d6) δ ppm 1.52-1.65 (m, 2 | 550 |
| | | H), 2.10-2.25 (m, 4 H), 2.37 (t, J = 7 Hz, 2 H), 2.53-2.71 | |
| | | (m partially hidden, 5 H), 2.78-2.94 (m, 2 H), 3.19 (br t, | |
| | | J = 7 Hz, 2 H), 3.76 (s, 3 H), 4.41 (dt, J = 47, 6 Hz, 2 H), 6.65- | |
| | | 7.06 (m, 8 H), 7.12 (d, J = 9 Hz, 1 H), 7.69 (dd, J = 8, 2 Hz, | |
| | | 1 H), 7.85 (d, J = 2 Hz, 1 H) | |
| 25 | A | 1H NMR (400 MHz, DMSO-d6) δ ppm 1.73-1.95 (m, 2 | 568 |
| | | H), 2.15 (br s, 4 H), 2.73-3.03 (m, 5 H), 3.12-3.23 (m, 2 | |
| | | H), 3.68-3.77 (m, 2 H), 3.78 (s, 3 H), 3.92-4.07 (m, 2 H), | |
| | | 4.49 (dt, J = 46, 5 Hz, 2 H), 6.78 (d, J = 8 Hz, 2 H), 6.83 (d, | |
| | | J = 8 Hz, 1 H), 6.95 (d, J = 8 Hz, 2 H), 7.02 (dd, J = 9, 3 Hz, 1 | |
| | | H), 7.10 (d, J-9 Hz, 1 H), 7.19 (d, J = 3 Hz, 1 H), 7.74 (dd, | |
| | | J = 8, 2 Hz, 1 H), 7.90 (d, J = 2 Hz, 1 H), 9.88-10.68 (m, 1 | |
| | | H), 12.85 (br s, 1 H) | |
| 26 | A | 1H NMR (400 MHz, DMSO-d6) δ ppm 1.75-1.93 (m, 2 | 552 |
| | | H), 2.16 (br quin, J = 7 Hz, 2 H), 2.29 (s, 3 H), 2.30-2.37 | |
| | | (m, 2 H), 2.81-2.92 (m, 5 H), 3.19 (br t, J = 7 Hz, 2 H), 3.65- | |
| | | 3.81 (m, 2 H), 3.83-4.06 (m, 2 H), 4.50 (dt, J = 47, 6 Hz, 2 | |
| | | H), 6.80 (d, J = 8 Hz, 2 H), 6.86 (d, J = 8 Hz, 1 H), 6.99 (d, | |
| | | J = 8 Hz, 2 H), 7.10 (s, 1 H), 7.30 (s, 1 H), 7.35 (s, 1 H), 7.74 | |
| | | (dd, J = 8, 2 Hz, 1 H), 7.91 (d, J = 2 Hz, 1 H), 10.23 (s, 1 H), | |
| | | 12.95 (s, 1 H) | |
| 27 | A | 1H NMR (400 MHz, DMSO-d6) δ ppm 1.67-1.83 (m, 2 | 554 |
| | | H), 2.09-2.22 (m, 2 H), 2.28 (t, J = 8 Hz, 2 H), 2.72-2.94 | |
| | | (m, 7 H), 3.30 (m hidden, 2 H), 3.67 (m, 2 H), 4.47 (dt, | |
| | | J = 47, 6 Hz, 2 H), 6.78 (d, J = 8 Hz, 2 H), 6.84 (d, J = 8 Hz, 1 | |
| | | H), 6.95 (d, J = 8 Hz, 2 H), 7.14-7.19 (m, 2 H), 7.23-7.28 | |
| | | (m, 2 H), 7.74 (dd, J = 8, 2 Hz, 1 H), 7.91 (d, J = 2 Hz, 1 H), | |
| | | 9.83-13.29 (m, 1 H) | |
| 28 | A | 1H NMR (400 MHz, DMSO-d6) δ ppm 1.62-1.78 (m, 2 | 56 |
| | | H), 2.06-2.22 (m, 4 H), 2.56-2.82 (m, 5 H), 2.89 (br t, | |
| | | J = 6 Hz, 2 H), 3.04-3.21 (m, 2 H), 3.46-3.68 (m, 2 H), | |
| | | 3.79 (s, 3 H), 4.45 (dt, J = 47, 6 Hz, 2 H), 6.73 (d, J = 8 Hz, 2 | |
| | | H), 6.86 (d, J-8 Hz, 1 H), 6.91 (d, J = 8 Hz, 2 H), 7.08-7.19 | |
| | | (m, 2 H), 7.51 (d, J = 9 Hz, 1 H), 7.74 (dd, J = 8, 2 Hz, 1 H), | |
| | | 7.91 (d, J = 2 Hz, 1 H), 10.87-13.46 (m, 1 H) | |
| 29 | A | 1H NMR (400 MHz, DMSO-d6) δ ppm 1.69-1.84 (m, 2 | 552 |
| | | H), 2.06-2.23 (m, 2 H), 2.26-2.32 (m, 2 H), 2.36 (br d, | |
| | | J = 2 Hz, 3 H), 2.71-2.93 (m, 7 H), 3.21-3.31 (m hidden, 2 | |
| | | H), 3.59-3.76 (m, 2 H), 4.48 (dt, J = 47, 6 Hz, 2 H), 6.79 (d, | |
| | | J = 8 Hz, 2 H), 6.85 (d, J = 8 Hz, 1 H), 6.99 (d, J = 8 Hz, 2 H), | |
| | | 7.25-7.32 (m, 2 H), 7.40 (dd, J = 8, 2 Hz, 1 H), 7.74 (dd, | |
| | | J = 8, 2 Hz, 1 H), 7.90 (d, J = 2 Hz, 1 H), 12.00 (br s, 1 H) | |
| 30 | A | 1H NMR (400 MHz, DMSO-d6) δ ppm 1.63-1.80 (m, 2 | 554 |
| | | H), 2.10-2.28 (m, 4 H), 2.59-2.78 (m, 5 H), 2.89 (br t, | |
| | | J = 7 Hz, 2 H), 3.10-3.22 (m, 2 H), 3.52-3.62 (m, 2 H), | |
| | | 4.45 (dt, J = 48, 6 Hz, 2 H), 6.72 (d, J = 8 Hz, 2 H), 6.84 (d, | |
| | | J = 8 Hz, 1 H), 6.92 (d, J = 8 Hz, 2 H), 7.16-7.38 (m, 4 H), | |
| | | 7.74 (dd, J = 8, 2 Hz, 1 H), 7.92 (d, J = 2 Hz, 1 H), 12.10 (br s, | |
| | | 1 H) | |
| 31 | A | 1H NMR (400 MHz, DMSO-d6) δ ppm 1.64-1.80 (m, 2 | 516 |
| | | H), 2.07 (s, 3 H), 2.09 (s, 3 H), 2.14 (m, 4 H), 2.68-2.82 | |
| | | (m, 5 H), 2.83-2.98 (m, 2 H), 3.13-3.28 (m, 2 H), 3.59 | |
| | | (m, 2 H), 4.46 (dt, J = 47, 6 Hz, 2 H), 6.71 (d, J = 8 Hz, 2 H), | |
| | | 6.79-7.02 (m, 5 H), 7.74 (dd, J = 8, 2 Hz, 1 H), 7.91 (d, J = 2 | |
| | | Hz, 1 H), 10.54-13.39 (m, 1 H) | |
| 32 | A | 1H NMR (400 MHz, DMSO-d6) δ ppm 0.65-0.86 (m, 2 | 578 |
| | | H), 0.90-1.07 (m, 2 H), 1.59-1.82 (m, 2 H), 1.99 (m, 1 | |
| | | H), 2.09-2.32 (m, 4 H), 2.63-2.78 (m, 5 H), 2.83-3.06 | |
| | | (m, 2 H), 3.09-3.20 (m, 2 H), 3.55 (br s, 2 H), 4.45 (dt, | |
| | | J = 48, 6 Hz, 2 H), 6.75-6.82 (m, 2 H), 6.87 (d, J = 8 Hz, 1 | |
| | | H), 6.92 (d, J = 8 Hz, 2 H), 7.01 (s, 1 H), 7.16 (d, J = 8 Hz, 1 | |
| | | H), 7.29 (dd, J = 8, 1 Hz, 1 H), 7.75 (dd, J = 8, 2 Hz, 1 H), | |
| | | 7.93 (d, J = 2 Hz, 1 H), 11.94 (br s, 1 H) | |
| 33 | A | 1H NMR (400 MHz, DMSO-d6) δ ppm 1.59-1.74 (m, 2 | 552 |
| | | H), 2.07-2.26 (m, 4 H), 2.21 (s, 3 H), 2.52-2.64 (m | |
| | | partially hidden, 5 H), 2.68-2.74 (m, 2 H), 2.90-3.03 (m, | |
| | | 4 H), 4.43 (dt, J = 47, 7 Hz, 2 H), 6.63-6.68 (m, 2 H), 6.79 | |
| | | (d, J = 8 Hz, 1 H), 6.87 (m, 2 H), 7.35 (t, J = 8 Hz, 1 H), 7.44 | |
| | | (d, J = 8 Hz, 1 H), 7.54 (d, J = 7 Hz, 1 H), 7.73 (dd, J = 8, 2 Hz, | |
| | | 1 H), 7.92 (d, J = 2 Hz, 1 H) | |
| 34 | A | 1H NMR (400 MHz, DMSO-d6) δ ppm 1.79-1.91 (m, 2 | 552 |
| | | H), 2.12-2.20 (m, 2 H), 2.29 (s, 3 H), 2.30-2.35 (m, 2 H), | |
| | | 2.81-2.92 (m, 5 H), 3.19 (br t, J = 7 Hz, 2 H), 3.67-3.81 | |
| | | (m, 2 H), 3.95 (s, 2 H), 4.50 (dt, J = 47, 6 Hz, 2 H), 6.80 (d, | |
| | | J = 8 Hz, 2 H), 6.86 (d, J = 8 Hz, 1 H), 6.99 (d, J = 8 Hz, 2 H), | |
| | | 7.10 (s, 1 H), 7.30 (s, 1 H), 7.35 (s, 1 H), 7.74 (dd, J = 8, 2 | |
| | | Hz, 1 H), 7.91 (d, J = 2 Hz, 1 H), 10.32 (br s, 1 H), 12.20- | |
| | | 13.74 (m, 1 H) | |
| 35 | B | 1H NMR (400 MHz, DMSO-d6) δ ppm 1.53-1.65 (m, 2 | 568 |
| | | H), 2.10-2.20 (m, 4 H), 2.37 (t, J = 7 Hz, 2 H), 2.55 (m | |
| | | hidden, 1 H), 2.61-2.72 (m, 4 H), 2.83 (m, 1 H), 2.98 (m, 1 | |
| | | H), 3.15-3.22 (m, 2 H), 3.66 (s, 3 H), 4.41 (dt, J = 47, 7 Hz, | |
| | | 2 H), 6.63 (d, J = 3 Hz, 1 H), 6.73-6.82 (m, 3 H), 6.88-6.95 | |
| | | (m, 3 H), 7.62 (d, J = 9 Hz, 1 H), 7.71 (dd, J = 8, 2 Hz, 1 H), | |
| | | 7.87 (d, J = 2 Hz, 1 H) | |
| 36 | B | 1H NMR (400 MHz, DMSO-d6) δ ppm 1.46-1.74 (m, 2 | 514 |
| | | H), 2.02-2.22 (m, 4 H), 2.40 (br t, J = 7 Hz, 2 H), 2.53- | |
| | | 2.77 (m, 5 H), 2.84 (br s, 1 H), 2.90-3.00 (m, 1 H), 3.16- | |
| | | 3.27 (m, 2 H), 4.35 (t, J = 6 Hz, 1 H), 4.47 (t, J = 6 Hz, 1 H), | |
| | | 5.54 (dt, J = 12, 2 Hz, 1 H), 5.83 (dt, J = 18, 1 Hz, 1 H), 6.67 | |
| | | (dd, J = 18, 12 Hz, 1 H), 6.73 (d, J = 8 Hz, 2 H), 6.80-6.86 | |
| | | (m, 2 H), 6.90 (d, J = 8 Hz, 2 H), 6.98-7.14 (m, 2 H), 7.74 | |
| | | (dd, J = 8, 2 Hz, 1 H), 7.89 (d, J = 2 Hz, 1 H) | |
| 37 | B | 1H NMR (400 MHz, DMSO-d6) δ ppm 1.02 (t, J = 8 Hz, 3 | 516 |
| | | H), 1.46-1.73 (m, 2 H), 2.09-2.23 (m, 4 H), 2.40 (t, J = 7 | |
| | | Hz, 2 H), 2.44-2.63 (m partially hidden, 3 H), 2.67 (d, J = 7 | |
| | | Hz, 2 H), 2.72 (br t, J = 7 Hz, 2 H), 2.80-2.96 (m, 2 H), 3.22 | |
| | | (br t, J = 7 Hz, 2 H), 4.41 (dt, J = 47, 6 Hz, 2 H), 6.70 (d, J = 7 | |
| | | Hz, 2 H), 6.84 (d, J = 8 Hz, 1 H), 6.86-6.92 (m, 3 H), | |
| | | 6.95(m, 1 H), 7.09 (td, J = 8, 6 Hz, 1 H), 7.73 (dd, J = 8, 2 Hz, | |
| | | 1 H), 7.90 (d, J = 2 Hz, 1 H) | |
| 38 | B | 1H NMR (400 MHz, DMSO-d6) δ ppm 1.02-1.18 (m, 3 | 532 |
| | | H), 1.52-1.67 (m, 2 H), 2.15-2.23 (m, 4 H), 2.40 (t, J = 8 | |
| | | Hz, 2 H), 2.51-2.81 (m partially hidden, 7 H), 2.85 (m, 1 | |
| | | H), 2.94 (m, 1 H), 3.22 (br t, J = 7 Hz, 2 H), 4.41 (dt, J = 47, 6 | |
| | | Hz, 2 H), 6.70 (d, J = 8 Hz, 2 H), 6.83-6.92 (m, 3 H), 7.01- | |
| | | 7.14 (m, 2 H), 7.25 (dd, J = 8, 2 Hz, 1 H), 7.74 (dd, J = 8, 2 | |
| | | Hz, 1 H), 7.91 (d, J = 2 Hz, 1 H), 10.72-14.09 (m, 1 H) | |
| 39 | A | 1H NMR (500 MHz, DMSO-d6) δ ppm 1.60-1.78 (m, 2 | 606 |
| | | H), 2.10-2.29 (m, 4 H), 2.64-2.75 (m, 5 H), 2.83-2.94 | |
| | | (m, 1 H), 2.97-3.07 (m, 1 H), 3.07-3.17 (m, 2 H), 3.45- | |
| | | 3.57 (m, 2 H), 4.44 (dt, J = 46, 6 Hz, 2 H), 6.75-6.78 (m, 2 | |
| | | H), 6.87 (d, J = 8 Hz, 1 H), 6.94 (m, 2 H), 7.47 (d, J = 8 Hz, 1 | |
| | | H), 7.76 (dd, J = 8, 2 Hz, 1 H), 7.85 (d, J = 8 Hz, 1 H), 7.93 | |
| | | (d, J = 2 Hz, 1 H), 8.04 (s, 1 H), 11.35-13.40 (m, 1 H) | |
| 40 | A | 1H NMR (400 MHz, DMSO-d6) δ ppm 1.69-1.88 (m, 2 | 518 |
| | | H), 2.08-2.24 (m, 4 H), 2.18 (s, 3 H), 2.73-2.86 (m, 3 H), | |
| | | 2.91-3.08 (m, 4 H), 3.30-3.54 (m partially hidden, 2 H), | |
| | | 3.71-3.84 (m, 2 H), 4.47 (dt, J = 47, 6 Hz, 2 H), 6.73-6.86 | |
| | | (m, 3 H), 6.92 (d, J = 8 Hz, 2 H), 7.04-7.16 (m, 2 H), 7.25 | |
| | | (dd, J = 8, 1 Hz, 1 H), 7.74 (dd, J = 8, 2 Hz, 1 H), 7.93 (d, J = 2 | |
| | | Hz, 1 H), 9.95-13.37 (m, 1 H) | |
| 41 | A | 1H NMR (400 MHz, DMSO-d6) δ ppm 1.63-1.83 (m, 2 | 502 |
| | | H), 2.09-2.24 (m, 4 H), 2.18 (s, 3 H), 2.64-2.80 (m | |
| | | partially hidden, 5 H), 2.87-3.00 (m, 2 H), 3.13-3.22 (m, | |
| | | 2 H), 3.59 (br s, 2 H), 4.45 (dt, J = 47, 6 Hz, 2 H), 6.75 (d, | |
| | | J = 8 Hz, 2 H), 6.85 (d, J = 8 Hz, 1 H), 6.88-6.99 (m, 4 H), | |
| | | 7.13 (td, J = 8, 6 Hz, 1 H), 7.75 (dd, J = 8, 2 Hz, 1 H), 7.93 (d, | |
| | | J = 2 Hz, 1 H), 9.99-13.71 (m, 2 H) | |
| 42 | A | 1H NMR (400 MHz, DMSO-d6) δ ppm 1.69-1.84 (m, 2 | 534 |
| | | H), 2.13 (m, 2 H), 2.26 (s, 3 H), 2.26-2.31 (m, 2 H), 2.73- | |
| | | 2.94 (m, 7 H), 3.20-3.26 (m hidden, 2 H), 3.70 (br s, 2 H), | |
| | | 4.47 (dt, J = 47, 6 Hz, 2 H), 6.80 (d, J = 9 Hz, 2 H), 6.84 (d, | |
| | | J = 8 Hz, 1 H), 7.06 (br t, J = 55 Hz, 1 H), 6.97 (d, J = 8 Hz, 2 | |
| | | H), 7.05 (d, J = 6 Hz, 1 H), 7.11 (s, 1 H), 7.29 (d, J = 8 Hz, 1 | |
| | | H), 7.74 (dd, J = 8, 2 Hz, 1 H), 7.90 (d, J = 2 Hz, 1 H) | |
| 43 | A | 1H NMR (500 MHz, DMSO-d6) δ ppm 1.61-1.77 (m, 2 | 572 |
| | | H), 2.12-2.22 (m, 4 H), 2.51-2.53 (m, 2 H), 2.65-2.71 | |
| | | (m, 1 H), 2.71-2.78 (m, 2 H), 2.86-2.95 (m, 2 H), 2.98- | |
| | | 3.17 (m, 2 H), 3.46-3.53 (m, 2 H), 4.44 (dt, J = 47, 6 Hz, 2 | |
| | | H), 6.73 (d, J = 8 Hz, 2 H), 6.86 (d, J = 8 Hz, 1 H), 6.94 (d, | |
| | | J = 9 Hz, 2 H), 7.09 (br d, J = 8 Hz, 1 H), 7.18 (t, J = 8 Hz, 1 | |
| | | H), 7.37-7.42 (m, 1 H), 7.76 (dd, J = 8, 2 Hz, 1 H), 7.93 (d, | |
| | | J = 2 Hz, 1 H), 11.41-13.70 (m, 1 H) | |
| 44 | A | 1H NMR (400 MHz, DMSO-d6) δ ppm 1.63-1.79 (m, 2 | 534 |
| | | H), 2.05-2.19 (m, 4 H), 2.64-2.78 (m, 5 H), 2.92-3.08 | |
| | | (m, 2 H), 3.13-3.22 (m, 2 H), 3.53-3.61 (m, 2 H), 3.68 (s, | |
| | | 3 H), 4.45 (dt, J = 47, 7 Hz, 2 H), 6.77 (d, J = 8 Hz, 2 H), 6.82 | |
| | | (d, J = 8 Hz, 1 H), 6.85-6.96 (m, 4 H), 7.17 (t, J = 8 Hz, 1 H), | |
| | | 7.72 (dd, J = 8, 2 Hz, 1 H), 7.90 (d, J = 2 Hz, 1 H), 9.94- | |
| | | 13.33 (m, 1 H) | |
| 45 | A | 1H NMR (400 MHz, DMSO-d6) δ ppm 1.58-1.76 (m, 2 H), | 518 |
| | | 2.03-2.18 (m, 4 H), 2.53-2.66 (m patially hidden, 3 H), | |
| | | 2.70-2.73 (m, 2 H), 2.84-2.95 (m, 2 H), 2.95-3.04 (m, 2 | |
| | | H), 3.44 (m, 2 H), 3.72 (s, 3 H), 4.44 (dt, J = 47, 6 Hz, 2 H), | |
| | | 6.62 (t, J = 8 Hz, 1 H), 6.74 (d, J = 8 Hz, 2 H), 6.79 (d, J = 9 Hz, | |
| | | 1 H), 6.84 (d, J = 8 Hz, 1 H), 6.89 (d, J = 8 Hz, 2 H), 7.18 (td, | |
| | | J = 8, 7 Hz, 1 H), 7.73 (dd, J = 8, 2 Hz, 1 H), 7.90 (d, J = 2 Hz, 1 | |
| | | H), 10.93-13.74 (m, 1 H) | |
| 46 | A | 1H NMR (400 MHz, DMSO-d6) δ ppm 1.61-1.76 (m, 2 | 530 |
| | | H), 2.07-2.22 (m, 4 H), 2.59-2.78 (m, 5 H), 2.80-2.93 | |
| | | (m, 2 H), 2.98-3.10 (m, 2 H), 3.44-3.51 (m, 2 H), 3.67 (s, | |
| | | 3 H), 3.77 (s, 3 H), 4.44 (dt, J = 46, 6 Hz, 2 H), 6.56 (dd, | |
| | | J = 7, 2 Hz, 1 H), 6.74-6.91 (m, 7 H), 7.73 (dd, J = 8, 2 Hz, 1 | |
| | | H), 7.89 (d, J = 2 Hz, 1 H) | |
| 47 | A | 1H NMR (400 MHz, DMSO-d6) δ ppm 1.67 (dquin, J = 27, | 522 |
| | | 6 Hz, 2 H), 2.06-2.23 (m, 4 H), 2.56-2.67 (m, 3 H), 2.70- | |
| | | 2.78 (m, 2 H), 2.86 (t, J = 7 Hz, 2 H), 2.96 (t, J = 7 Hz, 2 H), | |
| | | 3.42 (br t, J = 7 Hz, 2 H), 4.44 (dt, J = 47, 6 Hz, 2 H), 6.58 (td, | |
| | | J = 8, 6 Hz, 1 H), 6.77 (d, J = 8 Hz, 2 H), 6.80-6.91 (m, 2 H), | |
| | | 6.94 (d, J = 8 Hz, 2 H), 7.74 (dd, J = 8, 2 Hz, 1 H), 7.90 (d, | |
| | | J = 2 Hz, 1 H), 8.78-11.63 (m, 1 H) | |
| 48 | A | 1H NMR (400 MHz, DMSO-d6) δ ppm 1.79-1.93 (m, 2 | 514 |
| | | H), 2.12 (br s, 2 H), 2.21 (s, 3 H), 2.55-3.26 (m partially | |
| | | hidden, 9 H), 3.73-3.79 (m, 2 H), 3.74 (s, 3 H), 3.96-4.06 | |
| | | (m, 2 H), 4.50 (dt, J = 47, 6 Hz, 2 H), 6.73-6.79 (m, 2 H), | |
| | | 6.82-6.88 (m, 3 H), 6.92-6.98 (m, 2 H), 6.98-7.04 (m, 1 | |
| | | H), 7.74 (dd, J = 8, 2 Hz, 1 H), 7.90 (d, J = 2 Hz, 1 H), 9.55- | |
| | | 11.00 (m, 1 H), 12.19-13.50 (m, 1 H) | |
| 49 | A | 1H NMR (400 MHz, DMSO-d6) δ ppm 1.71-1.95 (m, 2 | 514 |
| | | H), 2.05 (s, 3 H), 2.04-2.20 (m, 4 H), 2.72-3.02 (m, 5 H), | |
| | | 3.04-3.17 (m, 2 H), 3.57-3.67 (m, 2 H), 3.69 (s, 3 H), | |
| | | 3.93 (br s, 2 H), 4.49 (dt, J = 47, 6 Hz, 2 H), 6.65 (d, J = 8 Hz, | |
| | | 1 H), 6.73-6.82 (m, 4 H), 6.89 (d, J = 8 Hz, 2 H), 7.06 (t, | |
| | | J = 8 Hz, 1 H), 7.72 (dd, J = 8, 2 Hz, 1 H), 7.91 (d, J = 2 Hz, 1 | |
| | | H), 9.58-11.22 (m, 1 H), 11.97-13.51 (m, 1 H) | |
| 50 | A | 1H NMR (400 MHz, DMSO-d6) δ ppm 1.67 (dquin, J = 27, | 588 |
| | | 6 Hz, 2 H), 2.17 (m, 4 H), 2.56-2.68 (m, 3 H), 2.70-2.75 | |
| | | (m, 2 H), 2.93-3.08 (m, 4 H), 3.38-3.52 (m, 2 H), 4.44 | |
| | | (dt, J = 47, 6 Hz, 2 H), 6.74 (d, J = 8 Hz, 2 H), 6.85 (d, J = 8 | |
| | | Hz, 1 H), 6.93 (d, J = 8 Hz, 2 H), 7.19 (d, J = 8 Hz, 1 H), 7.37 | |
| | | (t, J = 8 Hz, 1 H), 7.47 (d, J = 8 Hz, 1 H), 7.75 (dd, J = 8, 2 Hz, | |
| | | 1 H), 7.93 (d, J = 2 Hz, 1 H), 10.40-13.45 (m, 1 H) | |
| 51 | A | 1H NMR (400 MHz, DMSO-d6) δ ppm 1.74-1.90 (m, 2 | 530 |
| | | H), 1.99-2.13 (m, 4 H), 2.72-3.19 (m, 7 H), 3.25-3.28 | |
| | | (m, 2 H), 3.58 (s, 6 H), 3.88 (br s, 2 H), 4.48 (dt, J = 47, 6 | |
| | | Hz, 2 H), 6.52 (d, J = 8 Hz, 2 H), 6.73 (d, J = 8 Hz, 2 H), 6.81 | |
| | | (d, J = 8 Hz, 1 H), 6.87 (d, J = 8 Hz, 2 H), 7.11 (t, J = 8 Hz, 1 | |
| | | H), 7.71 (dd, J = 8, 2 Hz, 1 H), 7.88 (d, J = 2 Hz, 1 H), 9.20- | |
| | | 11.24 (m, 1 H), 12.20-13.53 (m, 1 H) | |
| 52 | A | 1H NMR (400 MHz, DMSO-d6) δ ppm 1.51-1.70 (m, 2 | 554 |
| | | H), 2.03-2.26 (m, 4 H), 2.37-2.44 (m, 2 H), 2.45-2.62 | |
| | | (m partially hidden, 1 H), 2.68-2.76 (m, 4 H), 2.81 (br t, | |
| | | J = 7 Hz, 2 H), 3.24 (t, J = 7 Hz, 2 H), 4.42 (dt, J = 47, 6 Hz, 2 | |
| | | H), 6.65 (d, J = 8 Hz, 1 H), 6.73-6.81 (m, 3 H), 6.84 (s, 1 | |
| | | H), 6.94 (d, J = 8 Hz, 2 H), 7.26 (d, J = 8 Hz, 1 H), 7.72 (dd, | |
| | | J = 8 Hz, 1 H), 7.88 (d, J = 2 Hz, 1 H), 9.92-11.42 (m, 1 H) | |
| 53 | A | 1H NMR (400 MHz, DMSO-d6) δ ppm 1.66-1.82 (m, 2 H), | 532 |
| | | 2.05-2.25 (m, 10 H), 2.71-2.88 (m, 5 H), 2.88-3.08 (m, 2 | |
| | | H), 3.10-3.50 (m partially hidden, 2 H), 3.60-3.74 (m, 2 | |
| | | H), 4.51 (dt, J = 47, 6 Hz, 2 H), 6.74-6.83 (m, 3 H), 6.86- | |
| | | 6.96 (m, 3 H), 7.08 (d, J = 2 Hz, 1 H), 7.73 (dd, J = 8, 2 Hz, 1 | |
| | | H), 7.92 (d, J = 2 Hz, 1 H) | |
| 54 | B | 1H NMR (400 MHz, DMSO-d6) δ ppm 1.78-1.92 (m, 2 H), | 534 |
| | | 2.02-2.30 (m, 4 H), 2.44 (s, 3 H), 2.79-3.02 (m, 5 H), 3.10- | |
| | | 3.21 (m, 2 H), 3.64-3.77 (m, 2 H), 3.90-4.03 (m, 2 H), | |
| | | 4.49 (dt, J = 47, 6 Hz, 2 H), 6.78 (d, J = 7 Hz, 2 H), 6.87 (d, | |
| | | J = 8 Hz, 1 H), 6.90-7.26 (m, 6 H), 7.76 (dd, J = 8, 2 Hz, 1 H), | |
| | | 7.92 (d, J = 2 Hz, 1 H), 10.13-10.89 (m, 1 H), 12.47-13.23 | |
| | | (m, 1 H) | |
| 55 | A | 1H NMR (400 MHz, DMSO-d6) δ ppm 1.76-1.96 (m, 2 H), | 528 |
| | | 2.00-2.09 (m, 2 H), 2.12 (s, 6 H), 2.14-2.24 (m, 2 H), 2.71- | |
| | | 3.01 (m, 5 H), 3.10-3.23 (m, 2 H), 3.68 (s, 3 H), 3.71- | |
| | | 3.86 (m, 2 H), 3.93-4.13 (m, 2 H), 4.49 (dt, J = 47, 6 Hz, 2 | |
| | | H), 6.55 (s, 2 H), 6.71 (d, J = 8 Hz, 2 H), 6.80 (d, J = 8 Hz, 1 | |
| | | H), 6.92 (d, J = 8 Hz, 2 H), 7.73 (dd, J = 8, 2 Hz, 1 H), 7.92 (d, | |
| | | J = 2 Hz, 1 H), 9.90-10.50 (m, 1 H), 12.63-12.94 (m, 1 H) | |
| 56 | A | 1H NMR (400 MHz, DMSO-d6) δ ppm 1.77-2.00 (m, 2 H), | 500 |
| | | 2.03 (s, 3 H), 2.07-2.16 (m, 2 H), 2.20 (m, 2 H), 2.75-2.88 | |
| | | (m, 3 H), 2.88-3.01 (m, 2 H), 3.14-3.27 (m, 2 H), 3.68- | |
| | | 3.87 (m, 2 H), 3.92-4.11 (m, 2 H), 4.41-4.61 (m, 2 H), | |
| | | 6.46 (dd, J = Hz, 1 H), 6.60 (d, J = 2 Hz, 1 H), 6.79-6.85 (m, 4 | |
| | | H), 6.90-7.03 (m, 2 H), 7.72 (dd, J = 8, 2 Hz, 1 H), 7.88 (d, | |
| | | J = 2 Hz, 1 H), 9.08 (s, 1 H), 10.38-10.92 (m, 1 H), 12.81 (br | |
| | | s, 1 H) | |
| 57 | A | 1H NMR (400 MHz, DMSO-d6) δ ppm 1.64-1.79 (m, 2 H), | 516 |
| | | 2.04 (d, J = 2 Hz, 3 H), 2.10-2.21 (m, 7 H), 2.70-2.79 (m, 5 | |
| | | H), 2.80-2.97 (m, 2 H), 3.07-3.21 (m, 2 H), 3.51-3.62 | |
| | | (m, 2 H), 4.45 (dt, J = 47, 6 Hz, 2 H), 6.72 (d, J = 8 Hz, 2 H), | |
| | | 6.77 (d, J = 8 Hz, 1 H), 6.84 (d, J = 8 Hz, 1 H), 6.87-7.00 (m, | |
| | | 3 H), 7.74 (dd, J = Hz, 1 H), 7.91 (d, J = 2 Hz, 1 H) | |
| 58 | A | 1H NMR (400 MHz, DMSO-d6) δ ppm 1.65-1.82 (m, 2 H), | 532 |
| | | 2.07-2.20 (m, 4 H), 2.23 (s, 3 H), 2.27 (s, 3 H), 2.68-2.79 | |
| | | (m, 5 H), 2.80-3.03 (m, 2 H), 3.09-3.22 (m, 2 H), 3.50- | |
| | | 3.63 (m, 2 H), 4.45 (dt, J = 47, 6 Hz, 2 H), 6.71 (d, J = 8 Hz, 2 | |
| | | H), 6.82-6.95 (m, 4 H), 7.02 (d, J = 8 Hz, 1 H), 7.74 (dd, | |
| | | J = Hz, 1 H), 7.91 (d, J = 2 Hz, 1 H), 10.70-12.78 (m, 1 H) | |
| 59 | B | 1H NMR (400 MHz, DMSO-d6) δ ppm 1.64-1.78 (m, 2 H), | 606 |
| | | 2.10-2.29 (m, 4 H), 2.68-2.79 (m, 5 H), 2.80-2.92 (m, 1 | |
| | | H), 2.94-3.09 (m, 1 H), 3.12-3.25 (m partially hidden, 2 | |
| | | H), 3.49-3.70 (m, 2 H), 4.45 (dt, J = 47, 6 Hz, 2 H), 6.77 (d, | |
| | | J = 8 Hz, 2 H), 6.86 (d, J = 8 Hz, 1 H), 6.96 (d, J = 8 Hz, 2 H), | |
| | | 7.52 (d, J = 8 Hz, 1 H), 7.64 (t, J = 8 Hz, 1 H), 7.76 (dd, | |
| | | J = 8Hz, 1 H), 7.87-7.95 (m, 2 H) | |
| 60 | B | 1H NMR (400 MHz, DMSO-d6) δ ppm 1.77-1.93 (m, 2 H), | 550 |
| | | 2.08-2.31 (m, 4 H), 2.78-3.00 (m, 5 H), 3.07-3.24 (m, 2 | |
| | | H), 3.63-3.74 (m, 2 H), 3.77 (s, 3 H), 3.88-4.06 (m, 2 H), | |
| | | 4.50 (dt, J = 47, 6 Hz, 1 H), 6.71-7.21 (m, 9 H), 7.74 (dd, | |
| | | J = 8, 2 Hz, 1 H), 7.91 (d, J = 2 Hz, 1 H), 10.06-10.95 (m, 1 | |
| | | H), 12.30-13.28 (m, 1 H) | |
| 61 | A | 1H NMR (400 MHz, DMSO-d6) δ ppm 1.79-1.93 (m, 2 H), | 500 |
| | | 1.99 (s, 3 H), 2.04-2.17 (m, 4 H), 2.73-2.99 (m, 5 H), 3.06- | |
| | | 3.21 (m, 2 H), 3.61-3.83 (m, 2 H), 3.89-4.09 (m, 2 H), | |
| | | 4.49 (dt, J = 47, 6 Hz, 2 H), 6.45 (d, J = 8 Hz, 1 H), 6.62 (d, | |
| | | J = 8 Hz, 1 H), 6.74 (d, J = 8 Hz, 2 H), 6.77-6.86 (m, 2 H), | |
| | | 6.91 (d, J = 8 Hz, 2 H), 7.73 (dd, J = 8, 2 Hz, 1 H), 7.90 (d, J = 2 | |
| | | Hz, 1 H), 9.21 (s, 1 H), 10.36-11.03 (m, 1 H), 12.49-13.06 | |
| | | (m, 1 H) | |
| 62 | B | 1H NMR (400 MHz, DMSO-d6) δ ppm 1.24 (s, 1 H), 1.50- | 552 |
| | | 1.68 (m, 2 H), 1.96 (t, J = 20 Hz, 3 H), 2.08-2.22 (m, 4 H), | |
| | | 2.38 (t, J = 7 Hz, 2 H), 2.47-2.57 (m partially hidden, 1H), | |
| | | 2.62-2.73 (m, 4 H), 2.77-3.02 (m, 2 H), 3.09-3.25 (m | |
| | | partially hidden, 2 H), 4.41 (dt, J-47, 6 Hz, 2 H), 6.74 (d, | |
| | | J = 8 Hz, 2 H), 6.82 (d, J = 8 Hz, 1 H), 6.90 (d, J = 8 Hz, 2 H), | |
| | | 7.05-7.14 (m, 2 H), 7.42 (dd, J = Hz, 1 H), 7.73 (dd, J = 8, 2 | |
| | | Hz, 1 H), 7.88 (d, J = 2 Hz, 1 H) | |
| 63 | A | 1H NMR (400 MHz, DMSO-d6) δ ppm 1.59-1.82 (m, 2 H), | 568 |
| | | 2.04-2.22 (m, 4 H), 2.52-2.82 (m, 5 H), 2.83-2.93 (m, 1 | |
| | | H), 2.97-3.25 (m partially hidden, 3 H), 3.48-3.62 (m, 2 | |
| | | H), 3.69 (s, 3 H), 4.45 (dt, J = 47, 6 Hz, 2 H), 6.75 (d, J = 8 Hz, | |
| | | 2 H), 6.83 (d, J = 8 Hz, 1 H), 6.88 (d, J = 8 Hz, 2 H), 7.18- | |
| | | 7.26 (m, 2 H), 7.40 (t, J = 8 Hz, 1 H), 7.73 (dd, J = 8, 2 Hz, 1 | |
| | | H), 7.91 (d, J = 2 Hz, 1 H) | |
| 64 | A | 1H NMR (400 MHz, DMSO-d6) δ ppm 1.68-1.91 (m, 2 H), | 623 |
| | | 2.08-2.27 (m, 7 H), 2.76-3.11 (m, 7 H), 3.48-3.65 (m, 2 | |
| | | H), 3.86 (m, 2 H), 4.49 (dt, J = 47, 6 Hz, 2 H), 6.76 (d, J = 8 | |
| | | Hz, 2 H), 6.87 (d, J = 8 Hz, 1 H), 6.97 (d, J = 8 Hz, 2 H), 7.10 | |
| | | (d, J = 8 Hz, 1 H), 7.45 (t, J = 8 Hz, 1 H), 7.77 (dd, J = Hz, 1 H), | |
| | | 7.95 (d, J = 2 Hz, 1 H), 9.38-13.65 (m, 2 H) | |
| 65 | A | 1H NMR (400 MHz, DMSO-d6) δ ppm 1.61-1.81 (m, 2 H), | 632 |
| | | 2.02-2.23 (m, 4 H), 2.10 (s, 3 H), 2.65-2.78 (m, 5 H), 2.90 | |
| | | (m, 1 H), 2.99 (m, 1 H), 3.07-3.21 (m, 2 H), 3.48-3.57 (m, | |
| | | 2 H), 3.63-3.67 (m, 3 H), 4.45 (dt, J = 47, 6 Hz, 2 H), 6.74 | |
| | | (d, J = 8 Hz, 2 H), 6.79-6.87 (m, 1 H), 6.85 (d, J = 8 Hz, 1 H), | |
| | | 6.92 (d, J = 8 Hz, 2 H), 7.00 (dd, J = Hz, 1 H), 7.75 (dd, J = Hz, | |
| | | 1 H), 7.93 (d, J = 2 Hz, 1 H) | |
| 66 | B | 1H NMR (400 MHz, DMSO-d6) δ ppm 1.62-1.82 (m, 2 H), | 534 |
| | | 2.06-2.27 (m, 4 H), 2.24 (s, 3 H), 2.67-2.85 (m, 5 H), 2.96 | |
| | | (br t, J = 7 Hz, 2 H), 3.11-3.30 (m partially hidden, 2 H), | |
| | | 3.55-3.71 (m, 2 H), 4.46 (dt, J = 47, 6 Hz, 2 H), 6.72 (d, J-8 | |
| | | Hz, 2 H), 6.85 (d, J = 8 Hz, 1 H), 6.91 (d, J = 8 Hz, 2 H), 6.93 | |
| | | (t, J = 55 Hz, 1 H), 7.26-7.48 (m, 3 H), 7.76 (dd, J = 8Hz, 1 | |
| | | H), 7.94 (d, J = 2 Hz, 1 H) | |
| 67 | G | 1H NMR (400 MHz, DMSO-d6) δ ppm 1.15 (t, J = 7 Hz, 3 | 566 |
| | | H), 1.62-1.84 (m, 2 H), 2.17-2.31 (m, 4 H), 2.39-3.05 | |
| | | (m, 9 H), 3.07-3.46 (m hidden, 2 H), 3.60-3.77 (m, 2 H), | |
| | | 4.47 (dt, J = 47, 6 Hz, 2 H), 6.69 (d, J = 8 Hz, 2 H), 6.86-6.95 | |
| | | (m, 3 H), 7.30 (t, J = 8 Hz 1 H), 7.41 (d, J = 8 Hz, 1 H), 7.56 | |
| | | (d, J = 8 Hz, 1 H), 7.76 (dd, J = Hz, 1 H), 7.94 (d, J = 2 Hz, 1 H) | |
| 68 | G | 1H NMR (400 MHz, DMSO-d6) δ ppm 1.21 (t, J = 7 Hz, 3 | 566 |
| | | H), 1.60-1.77 (m, 2 H), 1.99-2.27 (m, 4 H), 2.58-3.14 | |
| | | (m, 11 H), 3.14-3.70 (m hidden, 2 H), 4.44 (dt, J = 47, 6 Hz, | |
| | | 2 H), 6.77 (d, J = 8 Hz, 2 H), 6.84 (d, J = 8 Hz, 1 H), 6.89- | |
| | | 7.01 (m, 3 H), 7.24-7.43 (m, 2 H), 7.75 (dd, J = Hz, 1 H), | |
| | | 7.90 (d, J = 2 Hz, 1 H) | |
| 69 | G | 1H NMR (400 MHz, DMSO-d6) δ ppm 1.13 (t, J = 8 Hz, 3 | 566 |
| | | H), 1.70-1.89 (m, 2 H), 2.08-2.26 (m, 4 H), 2.39-3.18 | |
| | | (m, 9 H), 3.49-3.69 (m, 2 H), 3.79-3.99 (m, 2 H), 4.49 (dt, | |
| | | J = 47, 6 Hz, 2 H), 6.74 (d, J = 8 Hz, 2 H), 6.87 (d, J = 8 Hz, 1 | |
| | | H), 6.94 (d, J = 8 Hz, 2 H), 7.26 (d, J = 8 Hz, 1 H), 7.40 (dd, | |
| | | J = 8Hz, 1 H), 7.52 (t, J = 1 Hz, 1 H), 7.77 (dd, J = 8, 2 Hz, 1 H), | |
| | | 7.94 (d, J = 2 Hz, 1 H) | |
| 70 | G | 1H NMR (400 MHz, DMSO-d6) δ ppm 1.17 (t, J = 8 Hz, 3 | 566 |
| | | H), 1.67-1.86 (m, 2 H), 2.08-2.22 (m, 4 H), 2.59-2.65 | |
| | | (m, 2 H), 2.72-3.11 (m, 7 H), 3.35-3.57 (s partially | |
| | | hidden, 2 H), 3.57-3.86 (m, 5 H), 4.48 (dt, J = 47, 6 Hz, 2 | |
| | | H), 6.78 (d, J = 8 Hz, 2 H), 6.85 (d, J = 8 Hz, 1 H), 6.93 (d, J = 8 | |
| | | Hz, 2 H), 7.10 (d, J = 8 Hz, 1 H), 7.29 (br d, J = 8 Hz, 1 H), | |
| | | 7.54 (d, J = 1 Hz, 1 H), 7.75 (dd, J = Hz, 1 H), 7.92 (d, J = 2 Hz, | |
| | | 1 H), 10-13.5 (m, 1H) | |
| 71 | A | 1H NMR (400 MHz, DMSO-d6) δ ppm 1.73-1.92 (m, 2 H), | 530 |
| | | 2.07-2.20 (m, 2 H), 2.27-2.32 (m, 2 H), 2.77-2.97 (m, 5 | |
| | | H), 3.05-3.17 (m, 2 H), 3.43 (s partially hidden, 3 H), 3.59- | |
| | | 3.77 (m, 2 H), 3.71 (s, 3 H), 3.86-4.00 (m, 2 H), 4.50 (dt, | |
| | | J = 47, 6 Hz, 2 H), 6.63 (d, J = 2 Hz, 1 H), 6.71-6.86 (m, 5 H), | |
| | | 7.00 (d, J = 8 Hz, 2 H), 7.73 (dd, J = Hz, 1 H), 7.89 (d, J = 2 Hz, | |
| | | 1 H) | |
| 72 | A | 1H NMR (400 MHz, DMSO-d6) δ ppm 1.61-1.83 (m, 2 H), | 528 |
| | | 2.02 (s, 3 H), 2.00-2.18 (m, 4 H), 2.22 (s, 3 H), 2.68-2.85 | |
| | | (m, 5 H), 2.87-3.00 (m , 2 H), 3.13-3.32 (m partially | |
| | | hidden, 2 H), 3.54-3.65 (m, 2 H), 3.69 (s, 3 H), 4.46 (dt, | |
| | | J = 47, 6 Hz, 2 H), 6.47 (s, 1 H), 6.62 (s, 1 H), 6.75 (d, J = 8 | |
| | | Hz, 2 H), 6.80 (d, J = 8 Hz, 1 H), 6.89 (d, J = 8 Hz, 2 H), 7.72 | |
| | | (dd, J = Hz, 1 H), 7.90 (d, J = 2 Hz, 1 H) | |
| 73 | A | 1H NMR (400 MHz, DMSO-d6) δ ppm 1.65-1.83 (m, 2 H), | 516 |
| | | 2.05-2.28 (m, 4 H), 2.07 (d, J = 2 Hz, 3 H), 2.13 (s, 3 H), | |
| | | 2.72-2.88 (m, 5 H), 2.98 (br t, J = 7 Hz, 2 H), 3.11-3.35 (m | |
| | | partially hidden, 2 H), 3.60-3.83 (m, 2 H), 4.47 (dt, J = 47, 6 | |
| | | Hz, 2 H), 6.70 (d, J = 8 Hz, 2 H), 6.84 (d, J = 8 Hz, 1 H), 6.87- | |
| | | 6.94 (m, 3 H), 6.96-7.02 (m, 1 H), 7.75 (dd, J = 8, 2 Hz, 1 | |
| | | H), 7.94 (d, J = 2 Hz, 1 H) | |

The examples which follow describe the preparation of some compounds of formula (I) described herein. The numbers of the compounds exemplified below match those given in the Table 1a above. All reactions are performed under inert atmosphere, unless otherwise stated.

In the following examples, when the source of the starting products is not specified, it should be understood that said products are known compounds.

### INTERMEDIATES

### Intermediate 1: 3-(4-bromobenzyl)-1-(3-fluoropropyl)azetidine

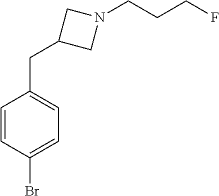 [SMILES: FCCCN1CC(Cc2ccc(Br)cc2)C1]

### Method 1

A suspension of 3-(4-bromobenzyl)azetidine, 2,2,2-trifluoroacetic acid (4.5 g, 13.23 mmol) in DMF (45 ml), K₂CO₃ (5.67 g, 41.01 mmol) and 1-fluoro-3-iodopropane (2.49 g, 13.32 mmol) was heated to 70° C. for 2 hours. After cooling to room temperature, water (500 ml) was added and the reaction mixture was extracted three times with 200 ml of EtOAc. The organic phases were gathered, washed with water (150 ml), dried over MgSO₄, filtered and concentrated under reduced pressure. The residue obtained was purified by flash chromatography, eluting with a gradient of DCM/MeOH: from 100/00 to 95/05 to give 2.3 g (61%) of 3-(4-bromobenzyl)-1-(3-fluoropropyl)azetidine as a viscous oil.

LC/MS (m/z, MH+): 286

### Method 2

A mixture of 3-(4-bromobenzyl)azetidine, 2,2,2-trifluoroacetic acid (4 g, 11.76 mmol) in THF (20 ml), 1-fluoro-3-iodopropane (2.21 g, 11.76 mmol) and NaOH 5N (7.06 ml, 35.28 mmol) was stirred at room temperature for 18 hours. The reaction mixture was concentrated under reduced pressure. To the resulting residue was added water (150 ml) and the reaction mixture was extracted three times with 150 ml of EtOAc. The organic phases were gathered, washed with water (150 ml), dried over MgSO₄, filtered and concentrated under reduced pressure. The residue obtained was purified by flash chromatography, eluting with a gradient of DCM/MeOH: from 100/00 to 95/05 to give 1.58 g (47%) of 3-(4-bromobenzyl)-1-(3-fluoropropyl)azetidine as a viscous oil.

### Intermediate 2: Methyl 9-(4,4,5,5-tetramethyl-1,3,2-dioxaborolan-2-yl)-6,7-dihydro-5H-benzo[7]annulene-3-carboxylate

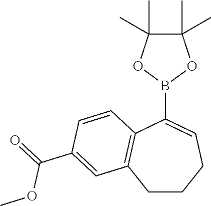

A mixture of methyl 9-(((trifluoromethyl)sulfonyl)oxy)-6,7-dihydro-5H-benzo[7]annulene-3-carboxylate (15 g, 42.82 mmol) (prepared according to WO2017140669), in toluene (150 ml), Pd(PPh₃)₂Cl₂ (1.53 g, 2.14 mmol), PPh₃ (673.87 mg, 2.57 mmol), bis(pinacolato)diboron (144.08 g, 52.67 mmol) and PhOK (8.04 g, 60.80 mmol) was heated to 75° C. during 1.5 hours. The yellow suspension becomes orange then brown. After cooling to room temperature, DCM (150 ml) and water (150 ml) were added, and decantation was done by hydrophobic column. The organic phase was concentrated under reduced pressure. The residue obtained was purified by flash chromatography, eluting with a gradient of heptane/DCM: from 85/15 to 20/80 to give 10.1 g (72%) of methyl 9-(4,4,5,5-tetramethyl-1,3,2-dioxaborolan-2-yl)-6,7-dihydro-5H-benzo[7]annulene-3-carboxylate as a white solid.

LC/MS (m/z, MH+): 329

### Intermediate 3: Methyl 8-bromo-9-(4-((1-(3-fluoropropyl)azetidin-3-yl)methyl)phenyl)-6,7-dihydro-5H-benzo[7]annulene-3-carboxylate

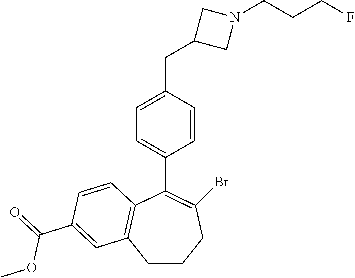 [SMILES: COC(=O)c1ccc2c(c1)CCCC(Br)=C2c1ccc(CC2CN(CCCF)C2)cc1]

### Step 1: Methyl 9-(4-((1-(3-fluoropropyl)azetidin-3-yl)methyl)phenyl)-6,7-dihydro-5H-benzo[7]annulene-3-carboxylate

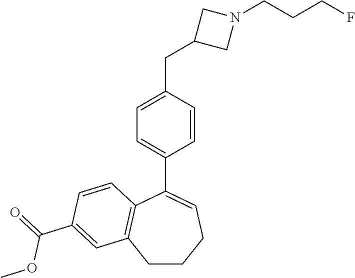 [SMILES: COC(=O)c1ccc2c(c1)CCCC=C2c1ccc(CC2CN(CCCF)C2)cc1]

A mixture of methyl 9-(4,4,5,5-tetramethyl-1,3,2-dioxaborolan-2-yl)-6,7-dihydro-5H-benzo[7]annulene-3-carboxylate (Intermediate 2) (17 g, 52.41 mmol), 3-(4-bromobenzyl)-1-(3-fluoropropyl)azetidine (Intermediate 1) (15 g, 52.41 mmol), Pd(dppf)Cl₂ complex with DCM (2.42 g, 3.14 mmol), Cs₂CO₃ (43.56 g, 134 mmol) in dioxane (120 ml) and water (50 ml) was heated to reflux for 1 hour. After cooling to room temperature, DCM (500 ml) and water (300 ml) were added, and decantation was done by hydrophobic column. The organic phase was concentrated under reduced pressure. The residue obtained was purified by flash chromatography, eluting with a gradient of DCM/MeOH: from 100/00 to 98/02 to give 16.19 g (90%) of methyl 9-(4-((1-(3-fluoropropyl)azetidin-3-yl)methyl)phenyl)-6,7-dihydro-5H-benzo[7]annulene-3-carboxylate as an orange viscous oil.

LC/MS (m/z, MH+): 408

### Step 2: Methyl 8-bromo-9-(4-((1-(3-fluoropropyl)azetidin-3-yl)methyl)phenyl)-6,7-dihydro-5H-benzo[7]annulene-3-carboxylate

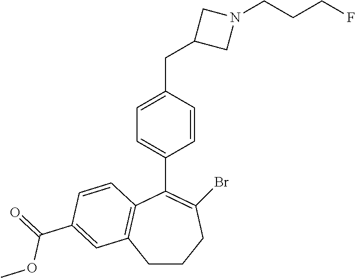 [SMILES: COC(=O)c1ccc2c(c1)CCCC(Br)=C2c1ccc(CC2CN(CCCF)C2)cc1]

To a mixture of methyl 9-(4-((1-(3-fluoropropyl)azetidin-3-yl)methyl)phenyl)-6,7-dihydro-5H-benzo[7]annulene-3-carboxylate (19 g, 46.62 mmol) in DCM (150 ml) was added pyridinium tribromide (21.54 g, 60.61 mmol). The reaction mixture was stirred for 2 hours at room temperature. Water (100 ml) and DCM (150 ml) were added and pH was adjusted to 8 with concentrated solution of NaHCO₃. The aqueous phase was washed 3 times with DCM and the gathered organic phases were dried over MgSO₄, filtered and concentrated under reduced pressure. The residue obtained was purified by flash chromatography eluting with a gradient of DCM/MeOH: from 100/00 to 95/05 to give 7.48 g (33%) of methyl 8-bromo-9-(4-((1-(3-fluoropropyl)azetidin-3-yl)methyl)phenyl)-6,7-dihydro-5H-benzo[7]annulene-3-carboxylate as a brown viscous oil.

LC/MS (m/z, MH+): 486

### Intermediate 4: Methyl 9-(4-((1-(3-fluoropropyl)azetidin-3-yl)methyl)phenyl)-8-(4,4,5,5-tetramethyl-1,3,2-dioxaborolan-2-yl)-6,7-dihydro-5H-benzo[7]annulene-3-carboxylate

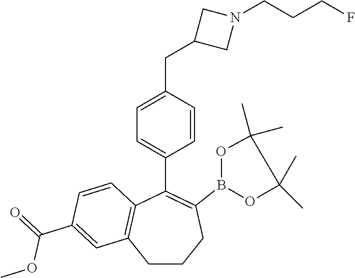 [SMILES: COC(=O)c1ccc2c(c1)CCCC(B1OC(C)(C)C(C)(C)O1)=C2c1ccc(CC2CN(CCCF)C2)cc1]

A mixture of methyl 8-bromo-9-(4-((1-(3-fluoropropyl)azetidin-3-yl)methyl)phenyl)-6,7-dihydro-5H-benzo[7]annulene-3-carboxylate (Intermediate 3) (9.7 g, 20 mmol) in toluene (150 ml), Pd(PPh₃)₂Cl₂ (1.5 g, 2 mmol), PPh₃ (1 g, 4 mmol), bis(pinacolato)diboron (13 g, 50 mmol) and PhOK (7.9 g, 60 mmol) was heated to 100° C. for 3 hours. After cooling to room temperature, a saturated solution of Na₂CO₃ (50 ml) was added. After decantation, the organic phase was washed with water (25 ml), dried over MgSO₄, filtered and concentrated under reduced pressure. The residue obtained was purified by flash chromatography, eluting with a gradient of DCM/MeOH: from 100/00 to 95/05 to give 8.2 g (75%) of methyl 9-(4-((1-(3-fluoropropyl)azetidin-3-yl)methyl)phenyl)-8-(4,4,5,5-tetramethyl-1,3,2-dioxaborolan-2-yl)-6,7-dihydro-5H-benzo[7]annulene-3-carboxylate.

LC/MS (m/z, MH+): 534

### Intermediate 5: 2-(3-((Benzyloxy)methyl)cyclobut-1-en-1-yl)-4,4,5,5-tetramethyl-1,3,2-dioxaborolane

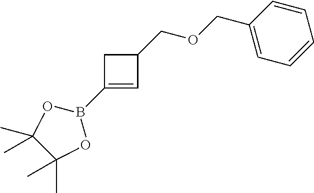 [SMILES: CC1(C)OB(C2=CC(COCc3ccccc3)C2)OC1(C)C]

### Step 1: 3-((Benzyloxy)methyl)cyclobut-1-en-1-yl trifluoromethanesulfonate

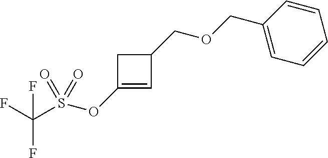 [SMILES: O=S(=O)(OC1=CC(COCc2ccccc2)C1)C(F)(F)F]

A solution of 3.9 ml (6.3 mmol) of butyllithium 1.6N in heptane was added to a solution of diisopropylamine (638 mg, 6.3 mmol) in 5 ml of THF at 0° C. under argon. To this mixture was slowly added a solution of 3-((benzyloxy)methyl)cyclobutan-1-one (1 g, 5.26 mmol) in 10 ml of THF at −78° C. under argon and the reaction mixture was stirred for 2 h. Then 1,1,1-trifluoro-N-phenyl-N-((trifluoromethyl)sulfonyl)methanesulfonamide (2.06 g, 5.78 mmol) were portionwise added and the mixture was allowed to raise to RT for 15 min then poured onto 50 ml of saturated aqueous NH₄Cl and 50 ml of Et₂O. The organic layer was washed with water and brine, dried over Na₂SO₄ and concentrated under reduced pressure. The residue was purified by flash chromatography eluting with a gradient of EtOAc in cyclohexane (100/0 to 95/05, v/v) to give 650 mg (39%) of 3-((benzyloxy)methyl)cyclobut-1-en-1-yl trifluoromethanesulfonate

LC/MS (m/z, MH+): 323.

### Step 2: 2-(3-((Benzyloxy)methyl)cyclobut-1-en-1-yl)-4,4,5,5-tetramethyl-1,3,2-dioxaborolane

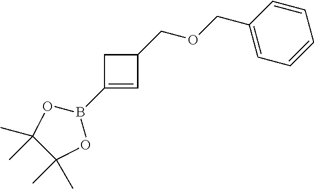 [SMILES: CC1(C)OB(C2=CC(COCc3ccccc3)C2)OC1(C)C]

To a solution of 3-((benzyloxy)methyl)cyclobut-1-en-1-yl trifluoromethanesulfonate (300 mg, 0.93 mmole), potassium acetate (274 mg, 2.79 mmol) and 4,4,4′,4′,5,5,5′,5′-octamethyl-2,2′-bi(1,3,2-dioxaborolane) (354 mg, 1.4 mmol) in anhydrous DMF (10 ml) under argon were added PdCl₂(dppf) (34 mg, 46 μmol). The mixture was stirred for 2 h at 80° C. then poured onto water and Et₂O (50 ml). The organic layer was washed with water and brine, dried over Na₂SO₄ and concentrated under reduced pressure. The residue obtained was used as such in the following step.

### Intermediate 6: Tert-butyldimethyl((3-(4,4,5,5-tetramethyl-1,3,2-dioxaborolan-2-yl)cyclopent-3-en-1-yl)methoxy)silane

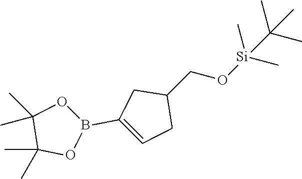 [SMILES: CC1(C)OB(C2=CCC(CO[Si](C)(C)C(C)(C)C)C2)OC1(C)C]

### Step 1: 4-(((Tert-butyldimethylsilyl)oxy)methyl)cyclopent-1-en-1-yl trifluoromethanesulfonate

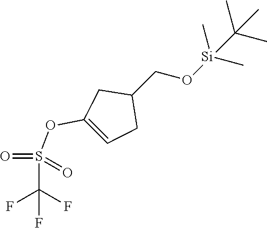 [SMILES: CC(C)(C)[Si](C)(C)OCC1CC=C(OS(=O)(=O)C(F)(F)F)C1]

Step 1 of Intermediate 6 was prepared following a similar procedure to that of step 1 of Intermediate 5 from 3-(((tert-butyldimethylsilyl)oxy)methyl)cyclopentan-1-one (Tetrahedron Assymetry (2013) 449-456) to give 4 g (56%) of 4-(((tert-butyldimethylsilyl)oxy)methyl)cyclopent-1-en-1-yl trifluoromethanesulfonate.

LC/MS (m/z, MH+): 361.

### Step 2: Tert-butyldimethyl((3-(4,4,5,5-tetramethyl-1,3,2-dioxaborolan-2-yl)cyclopent-3-en-1-yl)methoxy)silane

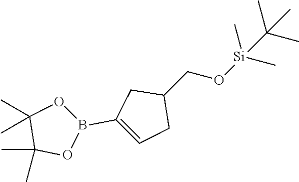 [SMILES: CC1(C)OB(C2=CCC(CO[Si](C)(C)C(C)(C)C)C2)OC1(C)C]

Step 2 of Intermediate 6 was prepared following a similar procedure to that of step 2 of Intermediate 5 from 4-(((tert-butyldimethylsilyl)oxy)methyl)cyclopent-1-en-1-yl trifluoromethanesulfonate to give 1.26 g (67%) of tert-butyldimethyl((3-(4,4,5,5-tetramethyl-1,3,2-dioxaborolan-2-yl)cyclopent-3-en-1-yl)methoxy)silane after purification by flash chromatography eluting with a gradient of EtOAc in cyclohexane (100/0 to 95/05, v/v).

LC/MS (m/z, MH+): 339.

### Intermediate 7: Methyl 8-(4,4-dimethylcyclohexyl)-9-(4,4,5,5-tetramethyl-1,3,2-dioxaborolan-2-yl)-6,7-dihydro-5H-benzo[7]annulene-3-carboxylate

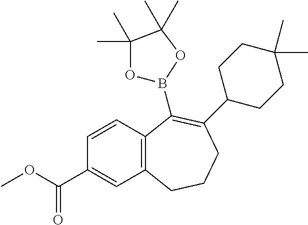

### Step 1: Methyl 6-bromo-5-oxo-6,7,8,9-tetrahydro-5H-benzo[7]annulene-2-carboxylate

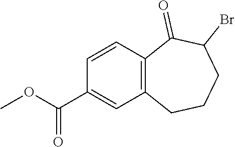 [SMILES: COC(=O)c1ccc2c(c1)CCCC(Br)C2=O]

To a mixture of methyl 5-oxo-6,7,8,9-tetrahydro-5H-benzo[7]annulene-2-carboxylate (9.42 g, 43.2 mmol) in DCM (400 mL) was portionwise added pyridinium tribromide (16.12 g, 45.4 mmol). The reaction mixture was stirred overnight at room temperature. Water (500 ml) and ethyl ether (1 L) were added. After decantation, the organic phase was washed twice with water, dried over MgSO₄, filtered and concentrated under reduced pressure to give 14.4 g (90%) of methyl 6-bromo-5-oxo-6,7,8,9-tetrahydro-5H-benzo[7]annulene-2-carboxylate.

LC/MS (m/z, MH+): 297

### Step 2: Methyl 9-acetoxy-8-bromo-6,7-dihydro-5H-benzo[7]annulene-3-carboxylate

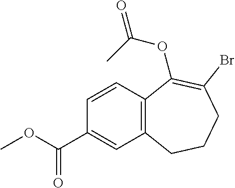 [SMILES: COC(=O)c1ccc2c(c1)CCCC(Br)=C2OC(C)=O]

To a solution of methyl 6-bromo-5-oxo-6,7,8,9-tetrahydro-5H-benzo[7]annulene-2-carboxylate (7.4 g, 25 mmol) in THF (80 mL) at −78° C. under Ar atmosphere was added LiHMDS (1 M, 27 mL). The mixture was stirred for 2 hours then treated with acetic anhydride (8.8 mL, 75 mmol) allowing the temperature to warmed up to 0° C. After pouring onto diisopropyl ether and water, the aqueous layer was separated and extracted with diisopropyl ether. After decantation, the combined organic layers were washed with water, dried over Na₂SO₄, filtered, and concentrated under reduced pressure. The residue was purified by flash chromatography with a 0-50% gradient of EtOAc in cyclohexane to give 6.97 g (83%) of methyl 9-acetoxy-8-bromo-6,7-dihydro-5H-benzo[7]annulene-3-carboxylate.

LC/MS (m/z, MH+): 339

### Step 3: Methyl 9-acetoxy-8-(4,4-dimethylcyclohex-1-en-1-yl)-6,7-dihydro-5H-benzol[7]annulene-3-carboxylate

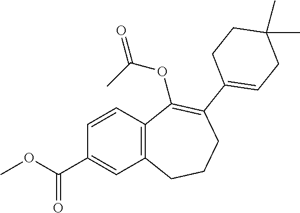

Step 3 of Intermediate 7 was prepared following a similar procedure to that of step 1 of Example 6 from methyl 9-acetoxy-8-bromo-6,7-dihydro-5H-benzo[7]annulene-3-carboxylate and 2-(4,4-dimethylcyclohex-1-en-1-yl)-4,4,5,5-tetramethyl-1,3,2-dioxaborolane to give 1.55 g (65%) of methyl 9-acetoxy-8-(4,4-dimethylcyclohex-1-en-1-yl)-6,7-dihydro-5H-benzo[7]annulene-3-carboxylate LC/MS (m/z, MH+): 369

### Step 4: Methyl 9-acetoxy-8-(4,4-dimethylcyclohexyl)-6,7-dihydro-5H-benzo[7]annulene-3-carboxylate

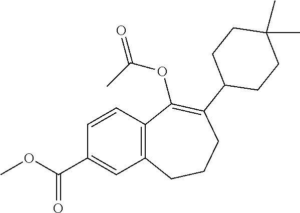

A mixture of methyl 9-acetoxy-8-(4,4-dimethylcyclohex-1-en-1-yl)-6,7-dihydro-5H-benzo[7]annulene-3-carboxylate (2.08 g, 5.65 mmol) and Pd/C 10% (400 mg) in EtOAc (50 ml) was hydrogenated at room temperature and 2.5 bars of H₂ for 5 hours. The reaction mixture was filtered. The filtrate was evaporated under reduced pressure to give 1.96 g (94%) of methyl 9-acetoxy-8-(4,4-dimethylcyclohexyl)-6,7-dihydro-5H-benzo[7]annulene-3-carboxylate LC/MS (m/z, MH+): 371

### Step 5: Methyl 6-(4,4-dimethylcyclohexyl)-5-oxo-6,7,8,9-tetrahydro-5H-benzo[7]annulene-2-carboxylate

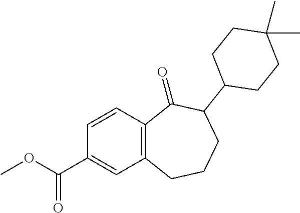

To a solution of methyl 9-acetoxy-8-(4,4-dimethylcyclohexyl)-6,7-dihydro-5H-benzo[7]annulene-3-carboxylate (1.96 g, 5.29 mmol) in MeOH (32 mL) and DCM (16 mL) was added a 12 N solution of HCl (5.29 mL, 63.5 mmol). The resulting reaction mixture was stirred overnight at room temperature. After pouring onto diethyl ether (20 ml), EtOAc (30 ml) and water (50 ml), the organic layer was separated, washed with water (50 ml), a 5% aqueous solution of Na₂CO₃ (50 ml) and water (50 ml) then dried over Na₂SO₄, filtered, and concentrated under reduced pressure. The residue obtained was purified by flash chromatography with a 0-20% gradient of Ethyl acetate in cyclohexane to give 1.59 g (87%) of methyl 6-(4,4-dimethylcyclohexyl)-5-oxo-6,7,8,9-tetrahydro-5H-benzo[7]annulene-2-carboxylate

LC/MS (m/z, MH+): 329

### Step 6: Methyl 8-(4,4-dimethylcyclohexyl)-9-(((trifluoromethyl)sulfonyl)oxy)-6,7-dihydro-5H-benzo[7]annulene-3-carboxylate

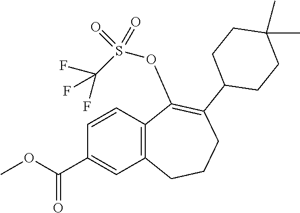

To a solution of methyl 6-(4,4-dimethylcyclohexyl)-5-oxo-6,7,8,9-tetrahydro-5H-benzo[7]annulene-2-carboxylate (1.35 g, 4.11 mmol) in THF (30 mL) at −55° C. under Ar atmosphere was added KHMDS (1 M, 4.93 ml, 4.93 mmol). After stirring at −55° C. for 30 minutes, N,N-bis(trifluoromethylsulfonyl)aniline (1.51 g, 4.23 mmol) was added. The reaction mixture was stirred for 30 minutes. Diethyl ether (50 ml) and an 5% aqueous solution of Na₂CO₃ (30 ml) were added. After decantation, the organic layer was dried over anhydrous Na₂SO₄, filtered, and concentrated under reduced pressure. The residue was purified by flash chromatography eluting with cyclohexane/EtOAc (95/05) to give 1.75 g (93%) of methyl 8-(4,4-dimethylcyclohexyl)-9-(((trifluoromethyl)sulfonyl)oxy)-6,7-dihydro-5H-benzo[7]annulene-3-carboxylate.

LC/MS (m/z, MH+): 461

### Step 7: Methyl 8-(4,4-dimethylcyclohexyl)-9-(4,4,5,5-tetramethyl-1,3,2-dioxaborolan-2-yl)-6,7-dihydro-5H-benzo[7]annulene-3-carboxylate

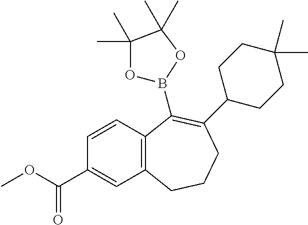

Step 7 of Intermediate 7 was prepared following a similar procedure to that of Intermediate 2 from methyl 8-(4,4-dimethylcyclohexyl)-9-(((trifluoromethyl)sulfonyl)oxy)-6,7-dihydro-5H-benzo[7]annulene-3-carboxylate to give 1.08 g (73%) of methyl 8-(4,4-dimethylcyclohexyl)-9-(4,4,5,5-tetramethyl-1,3,2-dioxaborolan-2-yl)-6,7-dihydro-5H-benzo[7]annulene-3-carboxylate

LC/MS (m/z, MH+): 439

### Intermediate 8: Tert-butyl 3-((6-chloro-5-fluoropyridin-3-yl)methylene)azetidine-1-carboxylate

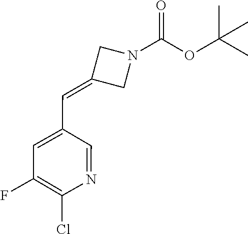 [SMILES: CC(C)(C)OC(=O)N1CC(=Cc2cnc(Cl)c(F)c2)C1]

To a mixture of 2-chloro-3-fluoro-5-iodopyridine (4.82 g, 18.7 mmol), tert-butyl 3-methyleneazetidine-1-carboxylate (3.17 g, 18.7 mmol), K₂CO₃ (5.18 g, 37.4 mmol) and t-butyl ammonium bromide (6 g, 18.7 mmol) in anhydrous DMF (100 ml) under argon were added palladium(II) acetate (420 mg, 1.87 mmol) and the mixture was heated at 55° C. for 16 h then cooled to RT. After dilution with Et₂O (500 ml), the mixture was washed with water (3×500 ml), dried over Na₂SO₄ and concentrated under reduced pressure. The residue was purified by flash chromatography eluting with a gradient of EtOAc in cyclohexane (100/0 to 0/100, v/v) to give 3.06 g (55%) of tert-butyl 3-((6-chloro-5-fluoropyridin-3-yl)methylene)azetidine-1-carboxylate after trituration in pentane.

LC/MS (m/z, MH+): 299

### Intermediate 9: 3,3-Difluoropropyl trifluoromethanesulfonate

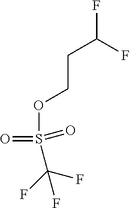 [SMILES: O=S(=O)(OCCC(F)F)C(F)(F)F]

To a solution of 3,3-difluoropropan-1-ol (1 g, 10.41 mmol) and 2,6-lutidine (2.66 mL, 22.9 mmol) in DCM (20 mL) at 0° C. was dropwise added trifluoromethanesulfonic anhydride (1.93 mL, 11.45 mmol). The mixture was stirred at 0° C. for 30 minutes. Ether and water were added. The aqueous layer was separated and extracted three times with ether. The combined organic layers were twice washed with a 10% aqueous solution of citric acid then water and dried over Na₂SO₄, filtered and concentrated under reduced pressure to give 2.06 g (86%) of 3,3-difluoropropyl trifluoromethanesulfonate which was used in the next step without further purification.

### Intermediate 10: Methyl 8-bromo-9-(4-((1-(3-fluoropropyl)azetidin-3-ylidene)methyl)phenyl)-6,7-dihydro-5H-benzo[7]annulene-3-carboxylate

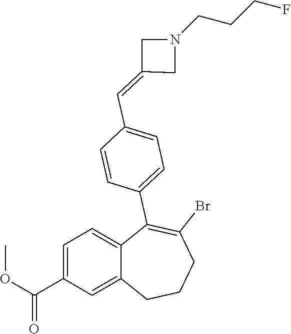 [SMILES: COC(=O)c1ccc2c(c1)CCCC(Br)=C2c1ccc(C=C2CN(CCCF)C2)cc1]

### Method 1

### Step 1: Methyl 9-(4-aminophenyl)-6,7-dihydro-5H-benzo[7]annulene-3-carboxylate

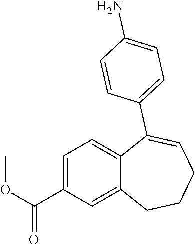 [SMILES: COC(=O)c1ccc2c(c1)CCCC=C2c1ccc(N)cc1]

A mixture of methyl 9-(((trifluoromethyl)sulfonyl)oxy)-6,7-dihydro-5H-benzo[7]annulene-3-carboxylate (20 g, 57.09 mmol) (prepared according to WO2017140669), 4-(4,4,5,5-tetramethyl-1,3,2-dioxaborolan-2-yl)aniline (13.13 g, 59.95 mmol), Cs₂CO₃ (37.21 g, 114.2 mmol), and Pd(dppf)Cl₂, complex with DCM (1.25 g, 1.71 mmol) in dioxane (160 mL) and water (40 mL) was heated to 95° C. for 1 hour. Water (200 mL) and EtOAc (500 mL) were added. After decantation, the organic phase was dried over MgSO₄, filtered, concentrated under reduced pressure and the residue obtained was purified by flash chromatography eluting with cyclohexane/EtOAc: 85/15 to give 14.5 g (87%) of methyl 9-(4-aminophenyl)-6,7-dihydro-5H-benzo[7]annulene-3-carboxylate.

LC/MS (m/z, MH+): 294

### Step 2: Methyl 9-(4-iodophenyl)-6,7-dihydro-5H-benzo[7]annulene-3-carboxylate

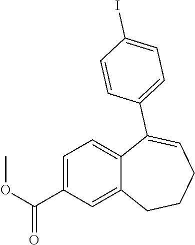 [SMILES: COC(=O)c1ccc2c(c1)CCCC=C2c1ccc(I)cc1]

To a mixture of methyl 9-(4-aminophenyl)-6,7-dihydro-5H-benzo[7]annulene-3-carboxylate (14.5 g, 49.4 mmol) in MeCN (270 mL) and 4N HCl (300 mL, 1200 mmol) cooled at 0° C., was slowly added a solution of sodium nitrite (3.58 g, 51.9 mmol) in water (20 mL). After stirring of the reaction mixture for 1 hour at 0° C., a solution of sodium iodide (14.8 g, 98.9 mmol) in water (40 mL) was added. The cooling bath was removed allowing the temperature to warm up to room temperature. After stirring for 4 hours at room temperature, Et₂O (500 mL) and a 2N solution of NaHSO₃ (200 mL) were added. After decantation, the organic phase was washed twice with water (100 mL), then with brine (100 mL), dried over MgSO₄, filtered, concentrated under reduced pressure and the residue obtained was purified by flash chromatography eluting with cyclohexane/EtOAc: 95/05 to give 14.8 g (74%) of methyl 9-(4-iodophenyl)-6,7-dihydro-5H-benzo[7]annulene-3-carboxylate.

LC/MS (m/z, MH+): 405

### Step 3: Methyl 8-bromo-9-(4-iodophenyl)-6,7-dihydro-5H-benzo[7]annulene-3-carboxylate

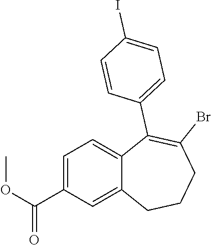 [SMILES: COC(=O)c1ccc2c(c1)CCCC(Br)=C2c1ccc(I)cc1]

To a mixture of methyl 9-(4-iodophenyl)-6,7-dihydro-5H-benzo[7]annulene-3-carboxylate (14.8 g, 36.6 mmol) in DCM (500 mL) was added pyridinium tribromide (12.9 g, 40.3 mmol). The reaction mixture was stirred for 18 hours at room temperature then diluted with Et₂O (500 mL) and pentane (500 mL) and washed with a 0.2N solution of NaHSO₃ (100 mL) and twice with water (200 mL). After decantation, the organic phase was dried over MgSO₄, filtered, concentrated under reduced pressure to give 17.7 g (100%) of methyl 8-bromo-9-(4-iodophenyl)-6,7-dihydro-5H-benzo[7]annulene-3-carboxylate.

LC/MS (m/z, MH+): 484

### Step 4: Tert-butyl 3-(4-(8-bromo-3-(methoxycarbonyl)-6,7-dihydro-5H-benzo[7]annulen-9-yl)benzylidene)azetidine-1-carboxylate

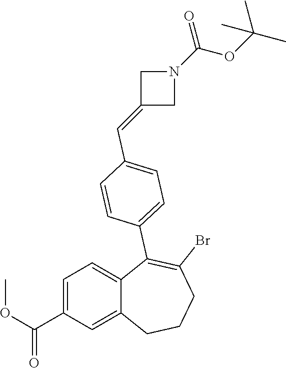 [SMILES: COC(=O)c1ccc2c(c1)CCCC(Br)=C2c1ccc(C=C2CN(C(=O)OC(C)(C)C)C2)cc1]

A solution of tert-butyl 3-methyleneazetidine-1-carboxylate (7.44 g, 44 mmol) and methyl 8-bromo-9-(4-iodophenyl)-6,7-dihydro-5H-benzo[7]annulene-3-carboxylate (17.7 g, 36.6 mmol) in DMF (200 mL) was degassed and purged with Ar for 5 min To this solution under stirring was added K₂CO₃ (10.1 g, 73.3 mmol), tetrabutylammonium bromide (11.8 g, 36.6 mmol) and palladium(II) acetate (0.83 g, 3.66 mmol). The mixture was heated to 50° C. for 30 hours then cooled to room temperature. Et₂O (300 mL) and water (300 mL) were added. After decantation, the aqueous phase was extracted with another 300 mL of Et₂O and the combined organic phases were washed twice with water (200 mL), dried over MgSO₄, filtered, concentrated under reduced pressure and purified by flash chromatography, eluting with a gradient of cyclohexane/EtOAc (95/05 to 80/20) to give 14.7 g (77%) tert-butyl 3-(4-(8-bromo-3-(methoxycarbonyl)-6,7-dihydro-5H-benzo[7]annulen-9-yl)benzylidene)azetidine-1-carboxylate.

LC/MS (m/z, MH+): 525

### Step 5: Methyl 9-(4-(azetidin-3-ylidenemethyl)phenyl)-8-bromo-6,7-dihydro-5H-benzo[7]annulene-3-carboxylate, 2,2,2-trifluoroacetic acid

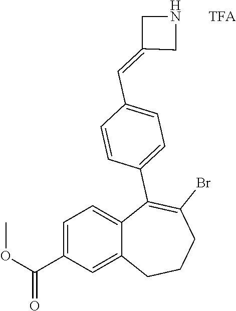 [SMILES: COC(=O)c1ccc2c(c1)CCCC(Br)=C2c1ccc(C=C2CNC2)cc1]

To a solution of tert-butyl 3-(4-(8-bromo-3-(methoxycarbonyl)-6,7-dihydro-5H-benzo[7]annulen-9-yl)benzylidene)azetidine-1-carboxylate (9.5 g, 18.1 mmol) in DCM (95 mL) was dropwise added TFA (24 mL, 0.33 mol). The reaction mixture was stirred at room temperature for 30 minutes and it was concentrated under reduced pressure. The residue was taken twice with DCM (30 ml) and concentrated under reduced pressure to give 10.4 g (100%) of methyl 9-(4-(azetidin-3-ylidenemethyl)phenyl)-8-bromo-6,7-dihydro-5H-benzo[7]annulene-3-carboxylate, 2,2,2-trifluoroacetic acid.

LC/MS (m/z, MH+): 425

### Step 6: Methyl 8-bromo-9-(4-((1-(3-fluoropropyl)azetidin-3-ylidene)methyl)phenyl)-6,7-dihydro-5H-benzo[7]annulene-3-carboxylate

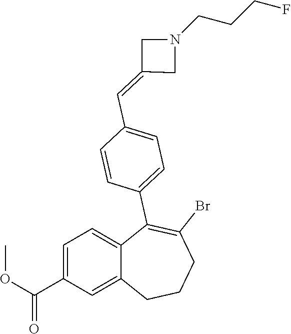 [SMILES: COC(=O)c1ccc2c(c1)CCCC(Br)=C2c1ccc(C=C2CN(CCCF)C2)cc1]

A mixture of 1-fluoro-3-iodopropane (2.88 g, 15.3 mmol) and methyl 9-(4-(azetidin-3-ylidenemethyl)phenyl)-8-bromo-6,7-dihydro-5H-benzo[7]annulene-3-carboxylate, 2,2,2-trifluoroacetic acid (8.24 g, 15.3 mmol) in a mixture of NaOH 1N (46 mL, 46 mmol) and DCM (70 mL) was stirred at room temperature for 48 hours. DCM (200 mL) and water (100 mL) were added. After decantation, the organic phase was dried over MgSO₄, filtered and concentrated under reduced pressure to give a residue. The residue obtained was purified by flash chromatography, eluting with a gradient of cyclohexane/EtOAc: from 100/00 to 00/100 to give 4.41 g (59% yield) of methyl 8-bromo-9-(4-((1-(3-fluoropropyl)azetidin-3-ylidene)methyl)phenyl)-6,7-dihydro-5H-benzo[7]annulene-3-carboxylate.

LC/MS (m/z, MH+): 485

### Method 2

### Step 1: Tert-butyl 3-(4-bromobenzoyl)azetidine-1-carboxylate

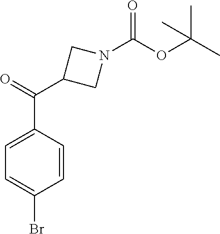 [SMILES: CC(C)(C)OC(=O)N1CC(C(=O)c2ccc(Br)cc2)C1]

To a solution of 1,4-dibromobenzene (290 g, 1.23 mol, 157 mL) in THF (1050 mL) was added n-BuLi (2.5 M, 491 mL) at −70° C. The mixture was stirred for 30 minutes before addition of tert-butyl 3-(methoxy(methyl)carbamoyl)azetidine-1-carboxylate (200 g, 819 mmol) in THF (420 mL) at −70° C. The reaction mixture was stirred for 1.5 hours. The solution was warmed up to −25° C. and slowly quenched by aqueous solution of saturated NH₄Cl (2000 mL). The mixture was extracted twice with MTBE (800 mL), dried over Na₂SO₄, filtered, and concentrated under reduced pressure. The obtained residue was purified by flash chromatography eluting with a gradient of petroleum ether/EtOAc from 10/1 to 0/1 to give 180 g (65%) of tert-butyl 3-(4-bromobenzoyl)azetidine-1-carboxylate as a white solid.

LC/MS (m/z, MH+): 340

### Step 2: Tert-butyl 3-(4-(3-(methoxycarbonyl)-6,7-dihydro-5H-benzo[7]annulen-9-yl)benzoyl)azetidine-1-carboxylate

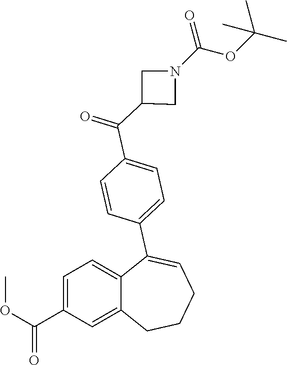 [SMILES: COC(=O)c1ccc2c(c1)CCCC=C2c1ccc(C(=O)C2CN(C(=O)OC(C)(C)C)C2)cc1]

Step 2 of Intermediate 10 (Method 2) was prepared following a similar procedure to that of step 1 of Intermediate 10 (Method 1) from methyl 9-(4,4,5,5-tetramethyl-1,3,2-dioxaborolan-2-yl)-6,7-dihydro-5H-benzo[7]annulene-3-carboxylate (Intermediate 3) and tert-butyl 3-(4-bromobenzoyl)azetidine-1-carboxylate to give 8.5 g (99%) of tert-butyl 3-(4-(3-(methoxycarbonyl)-6,7-dihydro-5H-benzo[7]annulen-9-yl)benzoyl)azetidine-1-carboxylate

LC/MS (m/z, MH+): 462

### Step 3: Tert-butyl 3-(4-(8-bromo-3-(methoxycarbonyl)-6,7-dihydro-5H-benzo[7]annulen-9-yl)benzoyl)azetidine-1-carboxylate

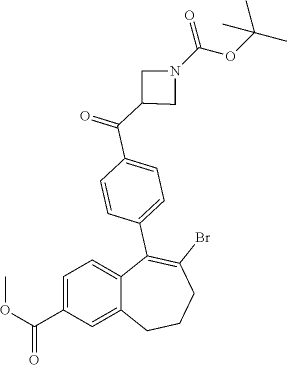 [SMILES: COC(=O)c1ccc2c(c1)CCCC(Br)=C2c1ccc(C(=O)C2CN(C(=O)OC(C)(C)C)C2)cc1]

Step 3 of Intermediate 10 (Method 2) was prepared following a similar procedure to that of step 3 of Intermediate 10 (Method 1) from tert-butyl 3-(4-(3-(methoxycarbonyl)-6,7-dihydro-5H-benzo[7]annulen-9-yl)benzoyl)azetidine-1-carboxylate to give 6.1 g (88%) of tert-butyl 3-(4-(8-bromo-3-(methoxycarbonyl)-6,7-dihydro-5H-benzo[7]annulen-9-yl)benzoyl)azetidine-1-carboxylate.

LC/MS (m/z, MH+): 540

### Step 4: Methyl 9-(4-(azetidine-3-carbonyl)phenyl)-8-bromo-6,7-dihydro-5H-benzo[7]annulene-3-carboxylate, 2,2,2-trifluoroacetic acid

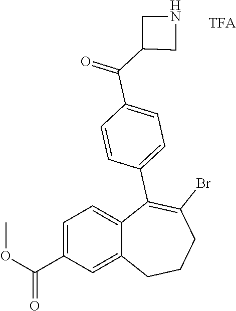 [SMILES: COC(=O)c1ccc2c(c1)CCCC(Br)=C2c1ccc(C(=O)C2CNC2)cc1]

Step 4 of Intermediate 10 (Method 2) was prepared following a similar procedure to that of step 5 of Intermediate 10 (Method 1) from tert-butyl 3-(4-(8-bromo-3-(methoxycarbonyl)-6,7-dihydro-5H-benzo[7]annulen-9-yl)benzoyl)azetidine-1-carboxylate to give 5 g (100%) of methyl 9-(4-(azetidine-3-carbonyl)phenyl)-8-bromo-6,7-dihydro-5H-benzo[7]annulene-3-carboxylate, 2,2,2-trifluoroacetic acid.

LC/MS (m/z, MH+): 440

### Step 5: Methyl 8-bromo-9-(4-(1-(3-fluoropropyl)azetidine-3-carbonyl)phenyl)-6,7-dihydro-5H-benzo[7]annulene-3-carboxylate

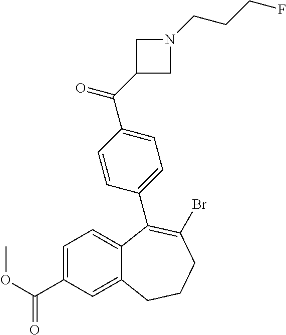 [SMILES: COC(=O)c1ccc2c(c1)CCCC(Br)=C2c1ccc(C(=O)C2CN(CCCF)C2)cc1]

A mixture of 1-fluoro-3-iodopropane (4.27 g, 22.7 mmol), methyl 9-(4-(azetidine-3-carbonyl)phenyl)-8-bromo-6,7-dihydro-5H-benzo[7]annulene-3-carboxylate, 2,2,2-trifluoroacetic acid (5 g, 9.24 mmol), K₂CO₃ (4.71 g, 34 mmol) in MeCN (200 mL) was heated to 70° C. for 1 hour. The reaction mixture was quenched by addition of water (200 mL), and then extracted with EtOAc (500 mL). After decantation, the organic phase was dried over MgSO₄, filtered, concentrated under reduced pressure, and the residue obtained was purified by flash chromatography, eluting with a gradient of cyclohexane/EtOAc: from 100/00 to 00/100 to give 3 g (53%) of methyl 8-bromo-9-(4-(1-(3-fluoropropyl)azetidine-3-carbonyl)phenyl)-6,7-dihydro-5H-benzo[7]annulene-3-carboxylate.

LC/MS (m/z, MH+): 500

### Step 6: Methyl 8-bromo-9-(4-((1-(3-fluoropropyl)azetidin-3-yl)(hydroxy)methyl)phenyl)-6,7-dihydro-5H-benzo[7]annulene-3-carboxylate

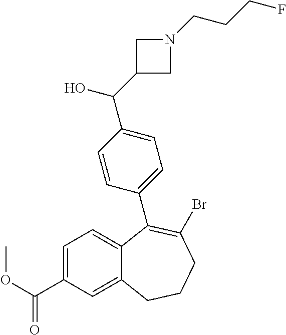 [SMILES: COC(=O)c1ccc2c(c1)CCCC(Br)=C2c1ccc(C(O)C2CN(CCCF)C2)cc1]

To a mixture of methyl 8-bromo-9-(4-(1-(3-fluoropropyl)azetidine-3-carbonyl)phenyl)-6,7-dihydro-5H-benzo[7]annulene-3-carboxylate (3 g, 6 mmol) in methanol (5 mL) cooled at 0° C. was added NaBH₄ (340 mg, 9 mmol). The reaction mixture was stirred at 0° C. for 30 minutes. 10% Citric acid aqueous solution (20 mL) and DCM (250 mL) were added. After decantation, the organic phase was dried over MgSO₄, filtered, concentrated under reduced pressure, and the residue obtained was purified by flash chromatography, eluting with a gradient of DCM/MeOH: from 100/00 to 05/95 to give 3 g (99%) of methyl 8-bromo-9-(4-((1-(3-fluoropropyl)azetidin-3-yl)(hydroxy)methyl)phenyl)-6,7-dihydro-5H-benzo[7]annulene-3-carboxylate.

LC/MS (m/z, MH+): 502

### Step 7: Methyl 8-bromo-9-(4-((1-(3-fluoropropyl)azetidin-3-ylidene)methyl)phenyl)-6,7-dihydro-5H-benzo[7]annulene-3-carboxylate

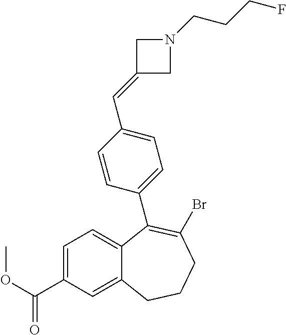 [SMILES: COC(=O)c1ccc2c(c1)CCCC(Br)=C2c1ccc(C=C2CN(CCCF)C2)cc1]

To a mixture of methyl 8-bromo-9-(4-((1-(3-fluoropropyl)azetidin-3-yl)(hydroxy)methyl)phenyl)-6,7-dihydro-5H-benzo[7]annulene-3-carboxylate (3 g, 5.97 mmol) in DCM (200 mL) were added pyridine (945 mg, 11.94 mmol, 0.96 mL) and trifluoromethylsulfonyl trifluoromethanesulfonate (3.37 g, 11.94 mmol, 2 mL). The reaction mixture was stirred at room temperature for 18 hours. DCM (400 mL) and a saturated aqueous solution of hydrogenocarbonate (300 mL) were added. After decantation, the organic phase was dried over MgSO₄, filtered and concentrated under reduced pressure to give a residue. The residue obtained was purified by flash chromatography, eluting with a gradient of DCM/MeOH: from 100/00 to 05/95 to give 1.9 g (66%) of methyl 8-bromo-9-(4-((1-(3-fluoropropyl)azetidin-3-ylidene)methyl)phenyl)-6,7-dihydro-5H-benzo[7]annulene-3-carboxylate.

LC/MS (m/z, MH+): 484

### Intermediate 11: Methyl 9-(4-((1-(3-fluoropropyl)azetidin-3-ylidene)methyl)phenyl)-8-(4,4,5,5-tetramethyl-1,3,2-dioxaborolan-2-yl)-6,7-dihydro-5H-benzo[7]annulene-3-carboxylate

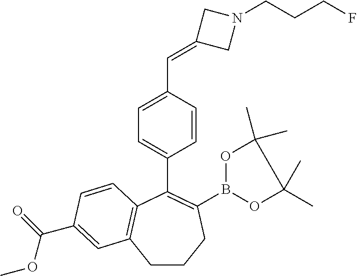 [SMILES: COC(=O)c1ccc2c(c1)CCCC(B1OC(C)(C)C(C)(C)O1)=C2c1ccc(C=C2CN(CCCF)C2)cc1]

A mixture of methyl 8-bromo-9-(4-((1-(3-fluoropropyl)azetidin-3-ylidene)methyl)phenyl)-6,7-dihydro-5H-benzo[7]annulene-3-carboxylate (Intermediate 10) (605 mg, 1.25 mmol) in toluene (30 mL), Pd(PPh₃)₂Cl₂ (35 mg, 50 μmol), PPh₃ (26 mg, 100 μmol), bis(pinacolato)diboron (793 mg, 3.12 mmol), K₂CO₃ (38 mg, 0.27 mmol) and PhOK (413 mg, 3.12 mmol) was degassed and purged with Ar for 5 min. then heated to 75° C. for 6 hours. After cooling to room temperature, Et₂O (100 mL) and a 5% solution of Na₂CO₃ (50 mL) were added. After decantation, the organic phase was washed with water (50 mL), dried over MgSO₄, filtered, concentrated under reduced pressure, and the residue obtained was purified by flash chromatography, eluting with a gradient of cyclohexane/EtOAc: from 100/00 to 00/100 to give 500 mg (75%) of methyl 9-(4-((1-(3-fluoropropyl)azetidin-3-ylidene)methyl)phenyl)-8-(4,4,5,5-tetramethyl-1,3,2-dioxaborolan-2-yl)-6,7-dihydro-5H-benzo[7]annulene-3-carboxylate.

LC/MS (m/z, MH+): 532

### EXAMPLES: COMPOUNDS OF FORMULA (I)

### Method A

### Example 6: 9-(4-((1-(3-Fluoropropyl)azetidin-3-yl)methyl)phenyl)-8-(2-methyl-3-(trifluoromethoxy)phenyl)-6,7-dihydro-5H-benzo[7]annulene-3-carboxylic acid

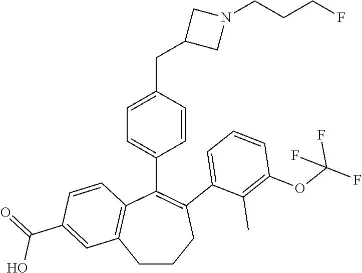 [SMILES: Cc1c(OC(F)(F)F)cccc1C1=C(c2ccc(CC3CN(CCCF)C3)cc2)c2ccc(C(=O)O)cc2CCC1]

### Step 1: Methyl 9-(4-((1-(3-fluoropropyl)azetidin-3-yl)methyl)phenyl)-8-(2-methyl-3-(trifluoromethoxy)phenyl)-6,7-dihydro-5H-benzo[7]annulene-3-carboxylate

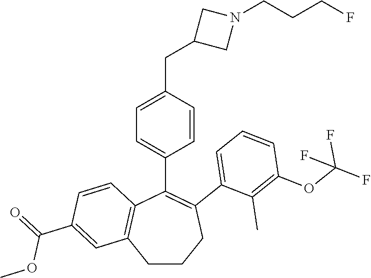 [SMILES: COC(=O)c1ccc2c(c1)CCCC(c1cccc(OC(F)(F)F)c1C)=C2c1ccc(CC2CN(CCCF)C2)cc1]

A mixture of methyl 8-bromo-9-(4-((1-(3-fluoropropyl)azetidin-3-yl)methyl)phenyl)-6,7-dihydro-5H-benzo[7]annulene-3-carboxylate (Intermediate 3) (100 mg, 205.6 μmol), 4,4,5,5-tetramethyl-2-[2-methyl-3-(trifluoromethoxy)phenyl]-1,3,2-dioxaborolane (93 mg, 308.4 μmol), Cs₂CO₃ (141 mg, 432 μmol), and Pd(dppf)Cl₂, complex with DCM (15 mg, 20.6 μmol) in dioxane (4 ml) and water (1 ml) was heated to reflux under microwave irradiation for 30 minutes. After cooling to room temperature, EtOAc (20 ml) and water (10 ml) were added. After decantation, the organic phase was dried over Na₂SO₄, filtered, and concentrated under reduced pressure. The residue obtained was purified by flash chromatography eluting with a gradient of MeOH in DCM (100/0 to 90/10, v/v) to give 48 mg (40%) of methyl 9-(4-((1-(3-fluoropropyl)azetidin-3-yl)methyl)phenyl)-8-(2-methyl-3-(trifluoromethoxy)phenyl)-6,7-dihydro-5H-benzo[7]annulene-3-carboxylate.

LC/MS (m/z, MH+): 582

### Step 2: 9-(4-((1-(3-Fluoropropyl)azetidin-3-yl)methyl)phenyl)-8-(2-methyl-3-(trifluoromethoxy)phenyl)-6,7-dihydro-5H-benzo[7]annulene-3-carboxylic acid

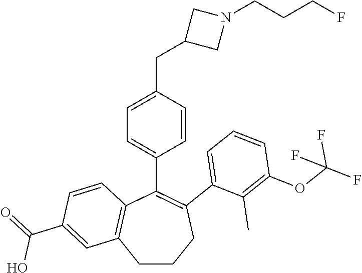 [SMILES: Cc1c(OC(F)(F)F)cccc1C1=C(c2ccc(CC3CN(CCCF)C3)cc2)c2ccc(C(=O)O)cc2CCC1]

To a solution of methyl 9-(4-((1-(3-fluoropropyl)azetidin-3-yl)methyl)phenyl)-8-(2-methyl-3-(trifluoromethoxy)phenyl)-6,7-dihydro-5H-benzo[7]annulene-3-carboxylate (48 mg, 82.5 μmol) in MeOH (5 ml) and water (1 ml) was added LiOH (8 mg, 330 PM) and the reaction mixture was stirred at room temperature for 18 hours. Water (10 ml), EtOAc (20 ml) and ethyl ether (20 ml) were added and pH was adjusted to 7 with HCl 0.1N. After decantation, the organic phase was dried over Na₂SO₄, filtered, and concentrated under reduced pressure. The residue obtained was purified by flash chromatography eluting with a gradient of MeOH in DCM (100/0 to 80/20, v/v) to give 46 mg (98%) of 9-(4-((1-(3-fluoropropyl)azetidin-3-yl)methyl)phenyl)-8-(2-methyl-3-(trifluoromethoxy)phenyl)-6,7-dihydro-5H-benzo[7]annulene-3-carboxylic acid.

### Example 46: 8-(2,3-Dimethoxyphenyl)-9-(4-((1-(3-fluoropropyl)azetidin-3-yl)methyl)phenyl)-6,7-dihydro-5H-benzo[7]annulene-3-carboxylic acid

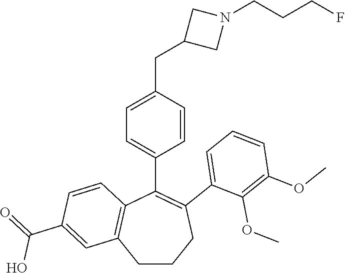 [SMILES: COc1cccc(C2=C(c3ccc(CC4CN(CCCF)C4)cc3)c3ccc(C(=O)O)cc3CCC2)c1OC]

### Step 1: Methyl 8-(2,3-dimethoxyphenyl)-9-(4-((1-(3-fluoropropyl)azetidin-3-yl)methyl)phenyl)-6,7-dihydro-5H-benzo[7]annulene-3-carboxylate

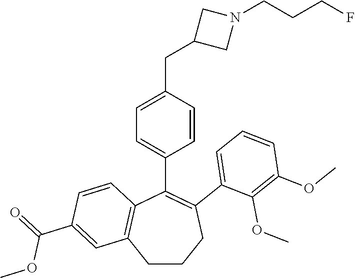 [SMILES: COC(=O)c1ccc2c(c1)CCCC(c1cccc(OC)c1OC)=C2c1ccc(CC2CN(CCCF)C2)cc1]

Step 1 of Example 46 was prepared following a similar procedure to that of step 1 of Example 6 from methyl 8-bromo-9-(4-((1-(3-fluoropropyl)azetidin-3-yl)methyl)phenyl)-6,7-dihydro-5H-benzo[7]annulene-3-carboxylate (Intermediate 3) and 2,3-dimethoxyphenylboronic acid to give 194 mg (87%) of methyl 8-(2,3-dimethoxyphenyl)-9-(4-((1-(3-fluoropropyl)azetidin-3-yl)methyl)phenyl)-6,7-dihydro-5H-benzo[7]annulene-3-carboxylate.

LC/MS (m/z, MH+): 544

### Step 2: 8-(2,3-Dimethoxyphenyl)-9-(4-((1-(3-fluoropropyl)azetidin-3-yl)methyl)phenyl)-6,7-dihydro-5H-benzo[7]annulene-3-carboxylic acid

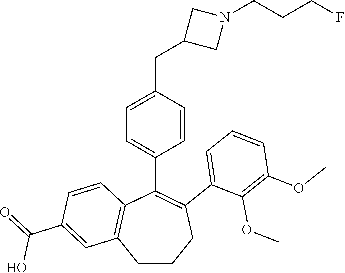 [SMILES: COc1cccc(C2=C(c3ccc(CC4CN(CCCF)C4)cc3)c3ccc(C(=O)O)cc3CCC2)c1OC]

Step 2 of example 46 was prepared following a similar procedure to that of step 2 of Example 6 from methyl 8-(2,3-dimethoxyphenyl)-9-(4-((1-(3-fluoropropyl)azetidin-3-yl)methyl)phenyl)-6,7-dihydro-5H-benzo[7]annulene-3-carboxylate to give 85.5 mg (45%) of 8-(2,3-dimethoxyphenyl)-9-(4-((1-(3-fluoropropyl)azetidin-3-yl)methyl)phenyl)-6,7-dihydro-5H-benzo[7]annulene-3-carboxylic acid.

### Method B

### Example 14: 8-(3-(Difluoromethoxy)-2-methylphenyl)-9-(4-((1-(3-fluoropropyl)azetidin-3-yl)methyl)phenyl)-6,7-dihydro-5H-benzo[7]annulene-3-carboxylic acid

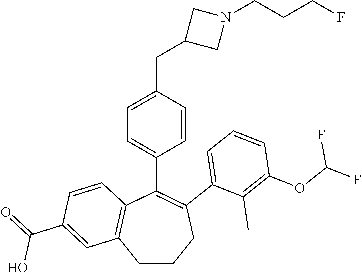 [SMILES: Cc1c(OC(F)F)cccc1C1=C(c2ccc(CC3CN(CCCF)C3)cc2)c2ccc(C(=O)O)cc2CCC1]

### Step 1: Methyl 8-(3-(difluoromethoxy)-2-methylphenyl)-9-(4-((1-(3-fluoropropyl)azetidin-3-yl)methyl)phenyl)-6,7-dihydro-5H-benzo[7]annulene-3-carboxylate

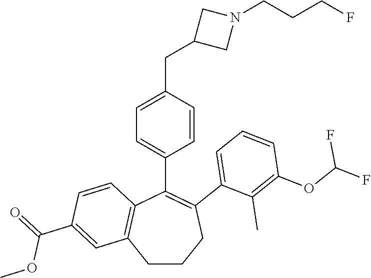 [SMILES: COC(=O)c1ccc2c(c1)CCCC(c1cccc(OC(F)F)c1C)=C2c1ccc(CC2CN(CCCF)C2)cc1]

Step 1 of Example 14 was prepared following a similar procedure to that of step 1 of Example 6 from methyl 9-(4-((1-(3-fluoropropyl)azetidin-3-yl)methyl)phenyl)-8-(4,4,5,5-tetramethyl-1,3,2-dioxaborolan-2-yl)-6,7-dihydro-5H-benzo[7]annulene-3-carboxylate (Intermediate 4) and 1-bromo-3-(difluoromethoxy)-2-methyl-benzene to give 99 mg (62%) of methyl 8-(3-(difluoromethoxy)-2-methylphenyl)-9-(4-((1-(3-fluoropropyl)azetidin-3-yl)methyl)phenyl)-6,7-dihydro-5H-benzo[7]annulene-3-carboxylate.

LC/MS (m/z, MH+): 564

### Step 2: 8-(3-(Difluoromethoxy)-2-methylphenyl)-9-(4-((1-(3-fluoropropyl)azetidin-3-yl)methyl)phenyl)-6,7-dihydro-5H-benzo[7]annulene-3-carboxylic acid

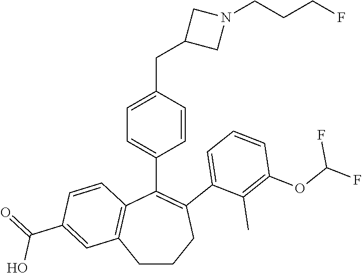 [SMILES: Cc1c(OC(F)F)cccc1C1=C(c2ccc(CC3CN(CCCF)C3)cc2)c2ccc(C(=O)O)cc2CCC1]

Step 2 of example 14 was prepared following a similar procedure to that of step 2 of Example 6 from methyl 8-(3-(difluoromethoxy)-2-methylphenyl)-9-(4-((1-(3-fluoropropyl)azetidin-3-yl)methyl)phenyl)-6,7-dihydro-5H-benzo[7]annulene-3-carboxylate to give 53 mg (55%) of 8-(3-(difluoromethoxy)-2-methylphenyl)-9-(4-((1-(3-fluoropropyl)azetidin-3-yl)methyl)phenyl)-6,7-dihydro-5H-benzo[7]annulene-3-carboxylic acid.

### Method C

### Example 15: 9-(4-((1-(3-Fluoropropyl)azetidin-3-yl)methyl)phenyl)-8-(3-hydroxypropyl)-6,7-dihydro-5H-benzo[7]annulene-3-carboxylic acid, 2,2,2-trifluoroacetic acid

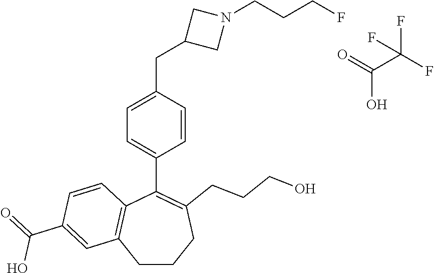

### Step 1: Methyl (E)-9-(4-((1-(3-fluoropropyl)azetidin-3-yl)methyl)phenyl)-8-(3-hydroxyprop-1-en-1-yl)-6,7-dihydro-5H-benzo[7]annulene-3-carboxylate

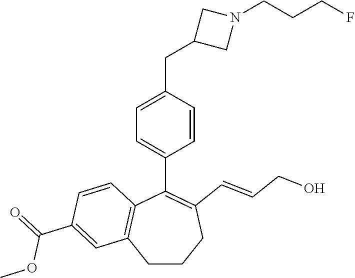 [SMILES: COC(=O)c1ccc2c(c1)CCCC(/C=C/CO)=C2c1ccc(CC2CN(CCCF)C2)cc1]

Step 1 of Example 15 was prepared following a similar procedure to that of step 1 of Example 6 from methyl 8-bromo-9-(4-((1-(3-fluoropropyl)azetidin-3-yl)methyl)phenyl)-6,7-dihydro-5H-benzo[7]annulene-3-carboxylate (Intermediate 3) and (E)-3-(4,4,5,5-tetramethyl-1,3,2-dioxaborolan-2-yl)prop-2-en-1-ol to give 140 mg (98%) of methyl (E)-9-(4-((1-(3-fluoropropyl)azetidin-3-yl)methyl)phenyl)-8-(3-hydroxyprop-1-en-1-yl)-6,7-dihydro-5H-benzo[7]annulene-3-carboxylate.

LC/MS (m/z, MH+): 464.

### Step 2: Methyl 9-(4-((1-(3-fluoropropyl)azetidin-3-yl)methyl)phenyl)-8-(3-hydroxypropyl)-6,7-dihydro-5H-benzo[7]annulene-3-carboxylate

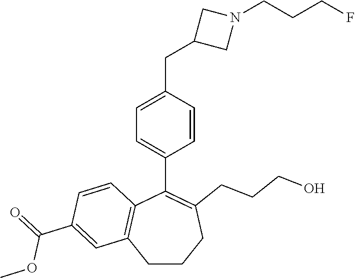 [SMILES: COC(=O)c1ccc2c(c1)CCCC(CCCO)=C2c1ccc(CC2CN(CCCF)C2)cc1]

A mixture of methyl (E)-9-(4-((1-(3-fluoropropyl)azetidin-3-yl)methyl)phenyl)-8-(3-hydroxyprop-1-en-1-yl)-6,7-dihydro-5H-benzo[7]annulene-3-carboxylate (100 mg, 215.7 μmol), Pd/C 10% (20 mg, 187 μmol) in DCM (10 ml) and MeOH (10 ml) was hydrogenated at room temperature and 4.5 bars of H₂ for 5 hours. The reaction mixture was filtered and the filtrate was evaporated under reduced pressure to give 99 mg (99%) of methyl 9-(4-((1-(3-fluoropropyl)azetidin-3-yl)methyl)phenyl)-8-(3-hydroxypropyl)-6,7-dihydro-5H-benzo[7]annulene-3-carboxylate.

LC/MS (m/z, MH+): 466

### Step 3: 9-(4-((1-(3-Fluoropropyl)azetidin-3-yl)methyl)phenyl)-8-(3-hydroxypropyl)-6,7-dihydro-5H-benzo[7]annulene-3-carboxylic acid, 2,2,2-trifluoroacetic acid

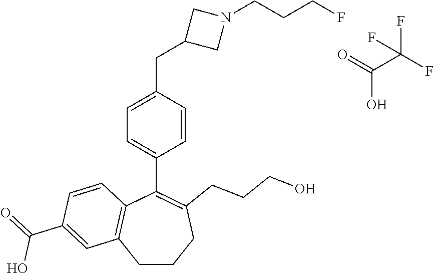

Step 3 of Example 15 was prepared following a similar procedure to that of step 2 of Example 6 from methyl 9-(4-((1-(3-fluoropropyl)azetidin-3-yl)methyl)phenyl)-8-(3-hydroxypropyl)-6,7-dihydro-5H-benzo[7]annulene-3-carboxylate to give 25 mg (21%) of 9-(4-((1-(3-fluoropropyl)azetidin-3-yl)methyl)phenyl)-8-(3-hydroxypropyl)-6,7-dihydro-5H-benzo[7]annulene-3-carboxylic acid, 2,2,2-trifluoroacetic acid.

### Method D

Example 21: 9-(4-((1-(3-Fluoropropyl)azetidin-3-yl)methyl)phenyl)-8-(3-(hydroxymethyl)cyclobutyl)-6,7-dihydro-5H-benzo[7]annulene-3-carboxylic acid

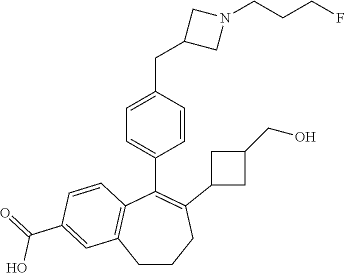 [SMILES: O=C(O)c1ccc2c(c1)CCCC(C1CC(CO)C1)=C2c1ccc(CC2CN(CCCF)C2)cc1]

### Step 1: Methyl 8-(3-((benzyloxy)methyl)cyclobut-1-en-1-yl)-9-(4-((1-(3-fluoropropyl)azetidin-3-yl)methyl)phenyl)-6,7-dihydro-5H-benzo[7]annulene-3-carboxylate

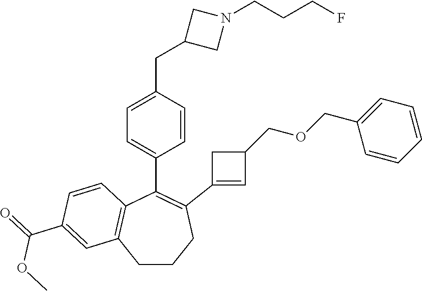 [SMILES: COC(=O)c1ccc2c(c1)CCCC(C1=CC(COCc3ccccc3)C1)=C2c1ccc(CC2CN(CCCF)C2)cc1]

Step 1 of Example 21 was prepared following a similar procedure to that of step 1 of Example 6 from methyl 8-bromo-9-(4-((1-(3-fluoropropyl)azetidin-3-yl)methyl)phenyl)-6,7-dihydro-5H-benzo[7]annulene-3-carboxylate (Intermediate 3) and 2-(3-((benzyloxy)methyl)cyclobut-1-en-1-yl)-4,4,5,5-tetramethyl-1,3,2-dioxaborolane (Intermediate 5) to give 278 mg (71%) of methyl 8-(3-((benzyloxy)methyl)cyclobut-1-en-1-yl)-9-(4-((1-(3-fluoropropyl)azetidin-3-yl)methyl)phenyl)-6,7-dihydro-5H-benzo[7]annulene-3-carboxylate.

LC/MS (m/z, MH+): 580.

### Step 2: Methyl 9-(4-((1-(3-fluoropropyl)azetidin-3-yl)methyl)phenyl)-8-(3-(hydroxymethyl)cyclobutyl)-6,7-dihydro-5H-benzo[₇]annulene-3-carboxylate

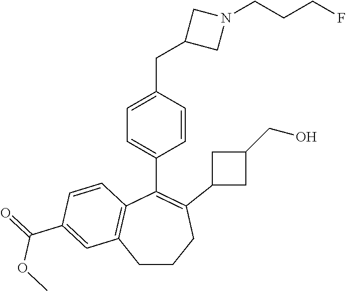 [SMILES: COC(=O)c1ccc2c(c1)CCCC(C1CC(CO)C1)=C2c1ccc(CC2CN(CCCF)C2)cc1]

A mixture of methyl 8-(3-((benzyloxy)methyl)cyclobut-1-en-1-yl)-9-(4-((1-(3-fluoropropyl)azetidin-3-yl)methyl)phenyl)-6,7-dihydro-5H-benzo[7]annulene-3-carboxylate (278 mg, 105 μmol), Pd/C 10% (250 mg, 235 μmol) in MeOH (20 ml) and DCM (20 ml) was hydrogenated at RT and 4 bars of H₂ for 20 hours. The reaction mixture was filtered, the filtrate was evaporated under reduced pressure and the residue was purified by flash chromatography eluting with a gradient of MeOH in DCM (100/0 to 97/03, v/v) to give 88 mg (37%) of methyl 9-(4-((1-(3-fluoropropyl)azetidin-3-yl)methyl)phenyl)-8-(3-(hydroxymethyl)cyclobutyl)-6,7-dihydro-5H-benzo[7]annulene-3-carboxylate.

LC/MS (m/z, MH+): 492

### Step 3: 9-(4-((1-(3-Fluoropropyl)azetidin-3-yl)methyl)phenyl)-8-(3-(hydroxymethyl)cyclobutyl)-6,7-dihydro-5H-benzo[7]annulene-3-carboxylic acid

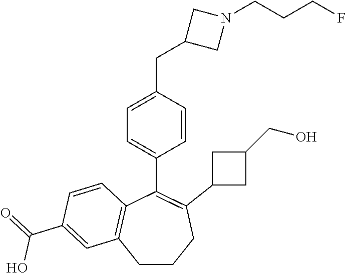 [SMILES: O=C(O)c1ccc2c(c1)CCCC(C1CC(CO)C1)=C2c1ccc(CC2CN(CCCF)C2)cc1]

Step 3 of Example 21 was prepared following a similar procedure to that of step 2 of Example 6 from methyl 9-(4-((1-(3-fluoropropyl)azetidin-3-yl)methyl)phenyl)-8-(3-(hydroxymethyl)cyclobutyl)-6,7-dihydro-5H-benzo[7]annulene-3-carboxylate to give 57 mg (67%) of 9-(4-((1-(3-fluoropropyl)azetidin-3-yl)methyl)phenyl)-8-(3-(hydroxymethyl)cyclobutyl)-6,7-dihydro-5H-benzo[7]annulene-3-carboxylic acid.

### Method E

### Example 3: 9-(4-((1-(3-Fluoropropyl)azetidin-3-yl)methyl)phenyl)-8-(3-(hydroxymethyl)cyclopentyl)-6,7-dihydro-5H-benzo[7]annulene-3-carboxylic acid

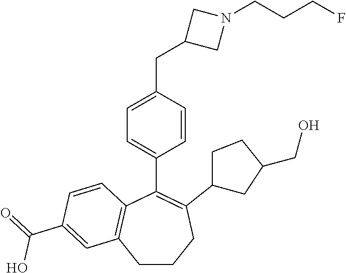 [SMILES: O=C(O)c1ccc2c(c1)CCCC(C1CCC(CO)C1)=C2c1ccc(CC2CN(CCCF)C2)cc1]

### Step 1: Methyl 8-(3-(((Tert-butyldimethylsilyl)oxy)methyl)cyclopent-1-en-1-yl)-9-(4-((1-(3-fluoropropyl)azetidin-3-yl)methyl)phenyl)-6,7-dihydro-5H-benzo[7]annulene-3-carboxylate

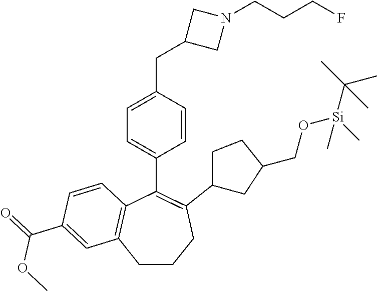 [SMILES: COC(=O)c1ccc2c(c1)CCCC(C1CCC(CO[Si](C)(C)C(C)(C)C)C1)=C2c1ccc(CC2CN(CCCF)C2)cc1]

Step 1 of Example 3 was prepared following a similar procedure to that of step 1 of Example 6 from methyl 8-bromo-9-(4-((1-(3-fluoropropyl)azetidin-3-yl)methyl)phenyl)-6,7-dihydro-5H-benzo[7]annulene-3-carboxylate (Intermediate 3) and tert-butyldimethyl((3-(4,4,5,5-tetramethyl-1,3,2-dioxaborolan-2-yl)cyclopent-2-en-1-yl)methoxy)silane (Intermediate 6) to give 770 mg (99%) of methyl 8-(3-(((tert-butyldimethylsilyl)oxy)methyl)cyclopent-1-en-1-yl)-9-(4-((1-(3-fluoropropyl)azetidin-3-yl)methyl)phenyl)-6,7-dihydro-5H-benzo[7]annulene-3-carboxylate.

LC/MS (m/z, MH+): 618.

### Step 2: Methyl 8-(3-(((tert-butyldimethylsilyl)oxy)methyl)cyclopentyl)-9-(4-((1-(3-fluoropropyl)azetidin-3-yl)methyl)phenyl)-6,7-dihydro-5H-benzo[7]annulene-3-carboxylate

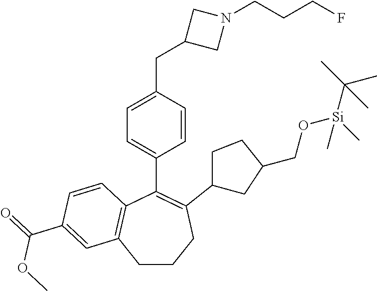 [SMILES: COC(=O)c1ccc2c(c1)CCCC(C1CCC(CO[Si](C)(C)C(C)(C)C)C1)=C2c1ccc(CC2CN(CCCF)C2)cc1]

A mixture of methyl 8-(3-(((tert-butyldimethylsilyl)oxy)methyl)cyclopent-1-en-1-yl)-9-(4-((1-(3-fluoropropyl)azetidin-3-yl)methyl)phenyl)-6,7-dihydro-5H-benzo[7]annulene-3-carboxylate (760 mg, 1.23 mmol), Pd/C 10% (80 mg, 75 μmol) in MeOH (15 ml) and DCM (15 ml) was hydrogenated at RT and 4 bars of H₂ for 7 hours. The reaction mixture was filtered and the filtrate was evaporated under reduced pressure to give 760 mg (99%) of methyl 8-(3-(((tert-butyldimethylsilyl)oxy)methyl)cyclopentyl)-9-(4-((1-(3-fluoropropyl)azetidin-3-yl)methyl)phenyl)-6,7-dihydro-5H-benzo[7]annulene-3-carboxylate

LC/MS (m/z, MH+): 620

### Step 3: Methyl 9-(4-((1-(3-fluoropropyl)azetidin-3-yl)methyl)phenyl)-8-(3-(hydroxymethyl)cyclopentyl)-6,7-dihydro-5H-benzo[7]annulene-3-carboxylate

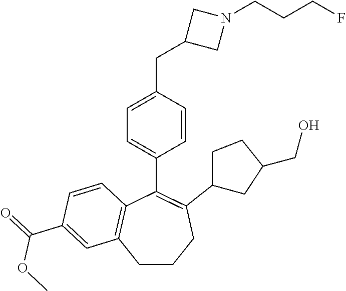 [SMILES: COC(=O)c1ccc2c(c1)CCCC(C1CCC(CO)C1)=C2c1ccc(CC2CN(CCCF)C2)cc1]

A mixture of methyl 8-(3-(((tert-butyldimethylsilyl)oxy)methyl)cyclopentyl)-9-(4-((1-(3-fluoropropyl)azetidin-3-yl)methyl)phenyl)-6,7-dihydro-5H-benzo[7]annulene-3-carboxylate (760 mg, 1.23 mmol) in 20 ml of acetonitrile and 20 ml of an aqueous 1N HCl solution was stirred at RT for 2 hours. The reaction mixture was neutralized with saturated aqueous NaHCO₃ and extracted with EtOAc (2×150 ml), dried over Na₂SO₄ and concentrated under reduced pressure. The residue was purified by flash chromatography eluting with a gradient of MeOH in DCM (100/0 to 95/05, v/v) to give 450 mg (73%) of methyl 9-(4-((1-(3-fluoropropyl)azetidin-3-yl)methyl)phenyl)-8-(3-(hydroxymethyl)cyclopentyl)-6,7-dihydro-5H-benzo[7]annulene-3-carboxylate.

LC/MS (m/z, MH+): 506

### Step 4: 9-(4-((1-(3-Fluoropropyl)azetidin-3-yl)methyl)phenyl)-8-(3-(hydroxymethyl)cyclopentyl)-6,7-dihydro-5H-benzo[7]annulene-3-carboxylic acid

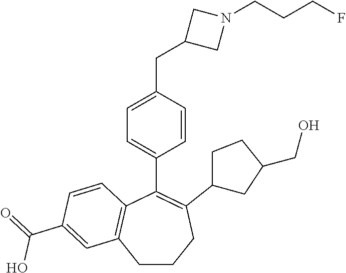 [SMILES: O=C(O)c1ccc2c(c1)CCCC(C1CCC(CO)C1)=C2c1ccc(CC2CN(CCCF)C2)cc1]

Step 4 of Example 3 was prepared following a similar procedure to that of step 2 of Example 6 from methyl 9-(4-((1-(3-fluoropropyl)azetidin-3-yl)methyl)phenyl)-8-(3-(hydroxymethyl)cyclopentyl)-6,7-dihydro-5H-benzo[7]annulene-3-carboxylate to give 16 mg (29%) of 9-(4-((1-(3-fluoropropyl)azetidin-3-yl)methyl)phenyl)-8-(3-(hydroxymethyl)cyclopentyl)-6,7-dihydro-5H-benzo[7]annulene-3-carboxylic acid.

### Method F

### Example 10: 8-(4,4-Dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylic acid

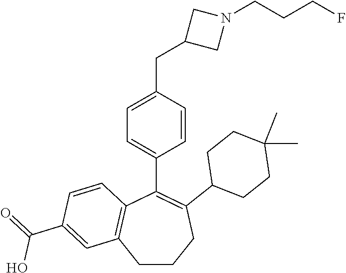 [SMILES: CC1(C)CCC(C2=C(c3ccc(CC4CN(CCCF)C4)cc3)c3ccc(C(=O)O)cc3CCC2)CC1]

Step 1: Tert-butyl 3-((6-(8-(4,4-dimethylcyclohexyl)-3-(methoxycarbonyl)-6,7-dihydro-5H-benzo[7]annulen-9-yl)-5-fluoropyridin-3-yl)methylene)azetidine-1-carboxylate

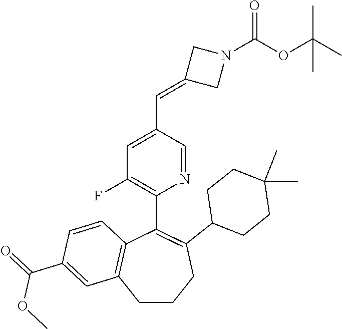 [SMILES: COC(=O)c1ccc2c(c1)CCCC(C1CCC(C)(C)CC1)=C2c1ncc(C=C2CN(C(=O)OC(C)(C)C)C2)cc1F]

A mixture of tert-butyl 3-((6-chloro-5-fluoropyridin-3-yl)methylene)azetidine-1-carboxylate (Intermediate 8) (715 mg, 2.4 mmol), methyl 8-(4,4-dimethylcyclohexyl)-9-(4,4,5,5-tetramethyl-1,3,2-dioxaborolan-2-yl)-6,7-dihydro-5H-benzo[7]annulene-3-carboxylate (Intermediate 7) (700 mg, 1.6 mmol), Cs₂CO₃ (1.56 g, 4.79 mmol) in 40 ml of toluene and 10 ml of water in a screw cap tube was degazed with argon for 5 min. CataCXium A Pd G3 ((di(1-adamantyl)-n-butylphosphine)-2-(2′-amino-1,1′-biphenyl)palladium(II) methanesulfonate, CAS number 1651823-59-4) (116 mg, 0.16 mmol) was added. The tube was sealed and the reacting mixture was stirred at 90° C. for 18 h. Water (10 ml) and Et₂O (30 ml) were added and the organic layer was washed with 10 ml of water, dried over Na₂SO₄ and concentrated under reduced pressure. The residue was purified by flash chromatography eluting with a gradient of EtOAc in cyclohexane (100/0 to 50/50, v/v) to give 560 mg (60%) of tert-butyl 3-((6-(8-(4,4-dimethylcyclohexyl)-3-(methoxycarbonyl)-6,7-dihydro-5H-benzo[7]annulen-9-yl)-5-fluoropyridin-3-yl)methylene)azetidine-1-carboxylate

LC/MS (m/z, MH+): 575

### Step 2: Methyl 9-(5-(azetidin-3-ylidenemethyl)-3-fluoropyridin-2-yl)-8-(4,4-dimethylcyclohexyl)-6,7-dihydro-5H-benzo[7]annulene-3-carboxylate, 2,2,2-trifluoroacetic acid

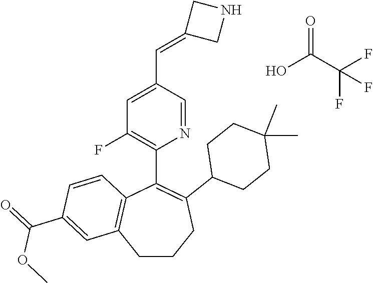

A mixture of tert-butyl 3-((6-(8-(4,4-dimethylcyclohexyl)-3-(methoxycarbonyl)-6,7-dihydro-5H-benzo[7]annulen-9-yl)-5-fluoropyridin-3-yl)methylene)azetidine-1-carboxylate (640 mg, 1.11 mmol) in DCM (4 ml) and trifluoroacetic acid (4 ml) was stirred at RT for 1 h then concentrated under reduced pressure to give 1.3 g of crude methyl 9-(5-(azetidin-3-ylidenemethyl)-3-fluoropyridin-2-yl)-8-(4,4-dimethylcyclohexyl)-6,7-dihydro-5H-benzo[7]annulene-3-carboxylate, 2,2,2-trifluoroacetic acid which was used as such in the next step.

LC/MS (m/z, MH+): 475.

### Step 3: Methyl 8-(4,4-dimethylcyclohexyl)-9-(3-fluoro-5-((1-(3-fluoropropyl)azetidin-3-ylidene)methyl)pyridin-2-yl)-6,7-dihydro-5H-benzo[7]annulene-3-carboxylate

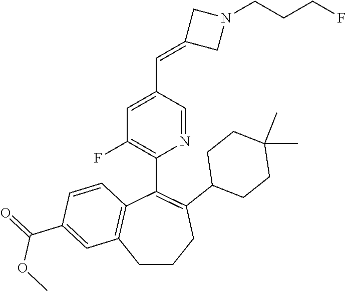 [SMILES: COC(=O)c1ccc2c(c1)CCCC(C1CCC(C)(C)CC1)=C2c1ncc(C=C2CN(CCCF)C2)cc1F]

A mixture of crude methyl 9-(5-(azetidin-3-ylidenemethyl)-3-fluoropyridin-2-yl)-8-(4,4-dimethylcyclohexyl)-6,7-dihydro-5H-benzo[7]annulene-3-carboxylate, 2,2,2-trifluoroacetic acid (370 mg, 0.47 mmole), 1-fluoro-3-iodopropane (97 mg, 0.52 mmole) and DIEA (244 mg, 1.89 mmol) in acetonitrile (10 ml) was stirred at RT for 20 h then half concentrated under reduced pressure and directly purified by flash chromatography eluting with a gradient of a mixture AcOEt/MeOH:80/20 in cyclohexane (100/0 to 0/100, v/v) to give 100 mg (40%) of methyl 8-(4,4-dimethylcyclohexyl)-9-(3-fluoro-5-((1-(3-fluoropropyl)azetidin-3-ylidene)methyl)pyridin-2-yl)-6,7-dihydro-5H-benzo[7]annulene-3-carboxylate.

LC/MS (m/z, MH+): 535

### Step 4: Methyl 8-(4,4-dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylate

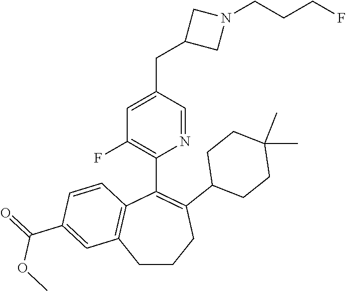 [SMILES: COC(=O)c1ccc2c(c1)CCCC(C1CCC(C)(C)CC1)=C2c1ncc(CC2CN(CCCF)C2)cc1F]

A mixture of methyl 8-(4,4-dimethylcyclohexyl)-9-(3-fluoro-5-((1-(3-fluoropropyl)azetidin-3-ylidene)methyl)pyridin-2-yl)-6,7-dihydro-5H-benzo[7]annulene-3-carboxylate (60 mg, 112 μmol), Pd/C 10% (60 mg, 56 μmol) in MeOH (10 ml) and AcOEt (5 ml) was hydrogenated at RT and 3 bars of H₂ for 11 hours. The reaction mixture was filtered and the filtrate was evaporated under reduced pressure to give 30 mg (50%) of methyl 8-(4,4-dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylate.

LC/MS (m/z, MH+): 537

### Step 5: 8-(4,4-Dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylic acid

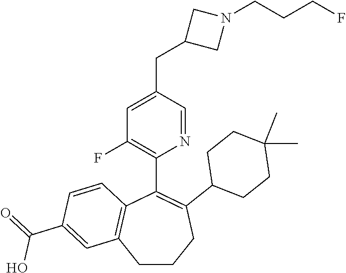 [SMILES: CC1(C)CCC(C2=C(c3ncc(CC4CN(CCCF)C4)cc3F)c3ccc(C(=O)O)cc3CCC2)CC1]

Step 5 of Example 10 was prepared following a similar procedure to that of step 2 of Example 6 from methyl 8-(4,4-dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylate to give 22 mg (75%) of 8-(4,4-dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylic acid.

### Example 13: 9-(5-((1-(3,3-Difluoropropyl)azetidin-3-yl)methyl)-3-fluoropyridin-2-yl)-8-(4,4-dimethylcyclohexyl)-6,7-dihydro-5H-benzo[7]annulene-3-carboxylic acid

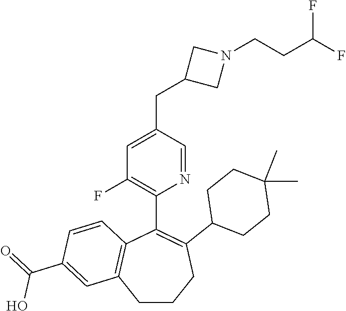 [SMILES: CC1(C)CCC(C2=C(c3ncc(CC4CN(CCC(F)F)C4)cc3F)c3ccc(C(=O)O)cc3CCC2)CC1]

### Step 1: Methyl 9-(5-((1-(3,3-difluoropropyl)azetidin-3-ylidene)methyl)-3-fluoropyridin-2-yl)-8-(4,4-dimethylcyclohexyl)-6,7-dihydro-5H-benzo[7]annulene-3-carboxylate

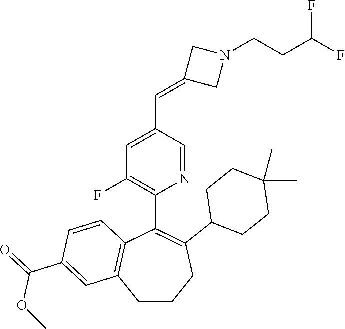 [SMILES: COC(=O)c1ccc2c(c1)CCCC(C1CCC(C)(C)CC1)=C2c1ncc(C=C2CN(CCC(F)F)C2)cc1F]

Step 1 of Example 13 was prepared following a similar procedure to that of step 3 of Example 10 from crude methyl 9-(5-(azetidin-3-ylidenemethyl)-3-fluoropyridin-2-yl)-8-(4,4-dimethylcyclohexyl)-6,7-dihydro-5H-benzo[7]annulene-3-carboxylate, 2,2,2-trifluoroacetic acid and 3,3-difluoropropyl trifluoromethanesulfonate (intermediate 9) to give 174 mg (46%) of methyl 9-(5-((1-(3,3-difluoropropyl)azetidin-3-ylidene)methyl)-3-fluoropyridin-2-yl)-8-(4,4-dimethylcyclohexyl)-6,7-dihydro-5H-benzo[7]annulene-3-carboxylate.

LC/MS (m/z, MH+): 553

### Step 2: 9-(5-((1-3,3-Difluoropropyl)azetidin-3-ylidene)methyl)-3-fluoropyridin-2-yl)-8-(4,4-dimethylcyclohexyl)-6,7-dihydro-5H-benzo[7]annulene-3-carboxylic acid

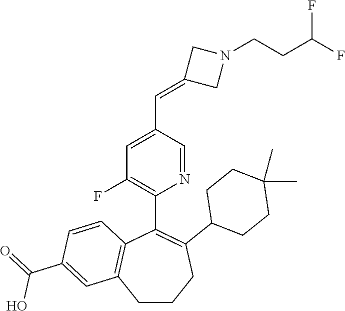 [SMILES: CC1(C)CCC(C2=C(c3ncc(C=C4CN(CCC(F)F)C4)cc3F)c3ccc(C(=O)O)cc3CCC2)CC1]

Step 2 of Example 13 was prepared following a similar procedure to that of step 2 of Example 6 from methyl 9-(5-((1-(3,3-difluoropropyl)azetidin-3-ylidene)methyl)-3-fluoropyridin-2-yl)-8-(4,4-dimethylcyclohexyl)-6,7-dihydro-5H-benzo[7]annulene-3-carboxylate to give 64 mg (33%) of 9-(5-((1-(3,3-difluoropropyl)azetidin-3-ylidene)methyl)-3-fluoropyridin-2-yl)-8-(4,4-dimethylcyclohexyl)-6,7-dihydro-5H-benzo[7]annulene-3-carboxylic acid

LC/MS (m/z, MH+): 539

### Step 3: 9-(5-((1-(3,3-Difluoropropyl)azetidin-3-yl)methyl)-3-fluoropyridin-2-yl)-8-(4,4-dimethylcyclohexyl)-6,7-dihydro-5H-benzo[7]annulene-3-carboxylic acid

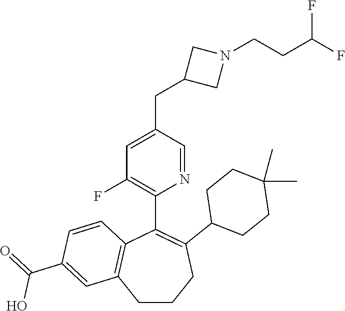 [SMILES: CC1(C)CCC(C2=C(c3ncc(CC4CN(CCC(F)F)C4)cc3F)c3ccc(C(=O)O)cc3CCC2)CC1]

Step 3 of Example 13 was prepared following a similar procedure to that of step 4 of Example 10 from 9-(5-((1-(3,3-difluoropropyl)azetidin-3-ylidene)methyl)-3-fluoropyridin-2-yl)-8-(4,4-dimethylcyclohexyl)-6,7-dihydro-5H-benzo[7]annulene-3-carboxylic acid to give 47 mg (46%) of 9-(5-((1-(3,3-difluoropropyl)azetidin-3-ylidene)methyl)-3-fluoropyridin-2-yl)-8-(4,4-dimethylcyclohexyl)-6,7-dihydro-5H-benzo[7]annulene-3-carboxylic acid.

### Method G

### Example 67: 8-(2-Ethyl-3-(trifluoromethyl)phenyl)-9-(4-((1-(3-fluoropropyl)azetidin-3-yl)methyl)phenyl)-6,7-dihydro-5H-benzo[7]annulene-3-carboxylic acid

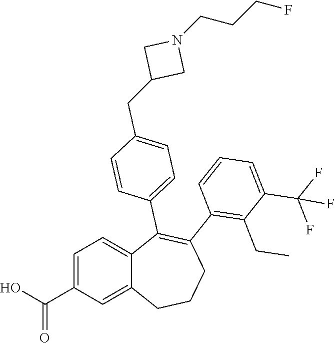 [SMILES: CCc1c(C2=C(c3ccc(CC4CN(CCCF)C4)cc3)c3ccc(C(=O)O)cc3CCC2)cccc1C(F)(F)F]

### Step 1: Methyl 8-(2-ethyl-3-(trifluoromethyl)phenyl)-9-(4-((1-(3-fluoropropyl)azetidin-3-ylidene)methyl)phenyl)-6,7-dihydro-5H-benzo[7]annulene-3-carboxylate

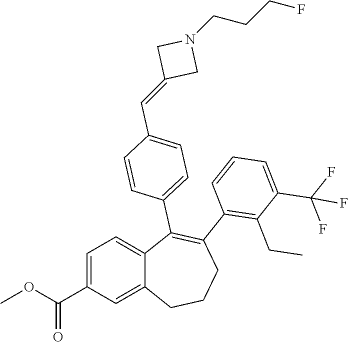 [SMILES: CCc1c(C2=C(c3ccc(C=C4CN(CCCF)C4)cc3)c3ccc(C(=O)OC)cc3CCC2)cccc1C(F)(F)F]

A mixture of methyl 9-(4-((1-(3-fluoropropyl)azetidin-3-ylidene)methyl)phenyl)-8-(4,4,5,5-tetramethyl-1,3,2-dioxaborolan-2-yl)-6,7-dihydro-5H-benzo[7]annulene-3-carboxylate (Intermediate 11) (200 mg, 338 μmol), 1-bromo-2-ethyl-3-(trifluoromethyl)benzene (129 mg, 508 μmol), Cs₂CO₃ (232 mg, 711 μmol), and Pd(dppf)Cl₂, complex with DCM (25 mg, 34 μmol) in dioxane (8 mL) and water (2 mL) was heated to 90° C. for 1 hour. After cooling to room temperature, addition of EtOAc (200 mL) and water (50 mL). After decantation, the organic phase was dried over MgSO₄, filtered concentrated under reduced pressure and the residue obtained was purified by flash chromatography eluting with a gradient of cyclohexane/EtOAc (100/0 to 0/100, v/v) to give 113 mg (58%) of methyl 8-(2-ethyl-3-(trifluoromethyl)phenyl)-9-(4-((1-(3-fluoropropyl)azetidin-3-ylidene)methyl)phenyl)-6,7-dihydro-5H-benzo[7]annulene-3-carboxylate.

LC/MS (m/z, MH+): 578

### Step 2: 8-(2-Ethyl-3-(trifluoromethyl)phenyl)-9-(4-((1-(3-fluoropropyl)azetidin-3-ylidene)methyl)phenyl)-6,7-dihydro-5H-benzo[7]annulene-3-carboxylic acid

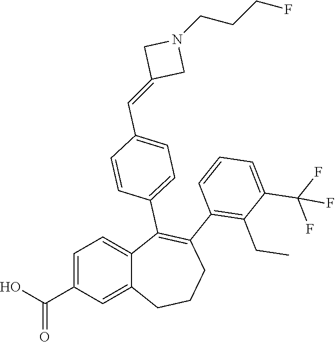 [SMILES: CCc1c(C2=C(c3ccc(C=C4CN(CCCF)C4)cc3)c3ccc(C(=O)O)cc3CCC2)cccc1C(F)(F)F]

Step 2 of Example 67 was prepare following a similar procedure to that of step 2 of Example 6 from methyl 8-(2-ethyl-3-(trifluoromethyl)phenyl)-9-(4-((1-(3-fluoropropyl)azetidin-3-ylidene)methyl)phenyl)-6,7-dihydro-5H-benzo[7]annulene-3-carboxylate to give 71 mg (64%) of 8-(2-ethyl-3-(trifluoromethyl)phenyl)-9-(4-((1-(3-fluoropropyl)azetidin-3-ylidene)methyl)phenyl)-6,7-dihydro-5H-benzo[7]annulene-3-carboxylic acid.

### Step 3: 8-(2-Ethyl-3-(trifluoromethyl)phenyl)-9-(4-((1-(3-fluoropropyl)azetidin-3-yl)methyl)phenyl)-6,7-dihydro-5H-benzo[7]annulene-3-carboxylic acid

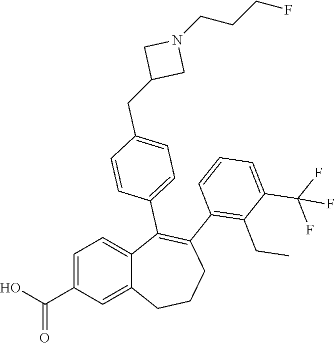 [SMILES: CCc1c(C2=C(c3ccc(CC4CN(CCCF)C4)cc3)c3ccc(C(=O)O)cc3CCC2)cccc1C(F)(F)F]

A mixture of 8-(2-ethyl-3-(trifluoromethyl)phenyl)-9-(4-((1-(3-fluoropropyl)azetidin-3-ylidene)methyl)phenyl)-6,7-dihydro-5H-benzo[7]annulene-3-carboxylic (20 mg, 35.5 μmol), Pd/C 10% (10 mg, 94 μmol) in DCM (5 ml) and MeOH (10 ml) was hydrogenated at room temperature and 5 bars of H₂ for 3 hours. The reaction mixture was filtered. The filtrate was evaporated under reduced pressure, triturated with diethyl ether/pentane then the solid was filtered and dried under vacuum to give 12 mg (60%) of 8-(2-ethyl-3-(trifluoromethyl)phenyl)-9-(4-((1-(3-fluoropropyl)azetidin-3-yl)methyl)phenyl)-6,7-dihydro-5H-benzo[7]annulene-3-carboxylic acid.

The compounds according to Table 1a above were subjected to pharmacological tests for determining their degradation effects on estrogen receptors.

### Test: Estrogen Receptor Degradation Activity

Said test involves measuring the in vitro degradation activity of the compounds of the Table 1a.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5p L at final concentrations ranging from 0.3-0.0000018 μM (in table 2), or 0.1 μM for fulvestrant (using as positive control). At 4 hours post compound addition the cells were fixed by adding 25 μL of formalin (final concentration 5% formalin containing 0.1% triton) for 10 minutes at room temperature and then washed twice with PBS. Then, 50 μL of LI-COR blocking buffer containing 0.1% Triton was added to plate for 30 minutes at room temperature. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or IC₅₀) in nM.

The % of ERα levels decrease were determined as follows: % inhibition=100*(1−(sample−fulvestrant: DMSO−fulvestrant)).

The Table 2 below indicates the estrogen receptor degradation activity results for the compounds of Table 1a tested at 0.3 μM, and demonstrates that said compounds have a significant degradation activity on estrogen receptors.

TABLE 2

| | | % |
|---|---|---|
| Compound | Degradation | Degradation |
| No. | IC₅₀ (nM) | At 0.3 μM |
| 1 | 0.5 | 85 |
| 2 | 0.6 | 86 |
| 3 | 62.3 | 77 |
| 4 | 0.6 | 83 |
| 5 | 0.4 | 87 |
| 6 | 0.5 | 90 |
| 7 | 0.6 | 93 |
| 8 | 1.5 | 89 |
| 9 | 6.1 | 88 |
| 10 | 3.6 | 83 |
| 11 | 0.9 | 86 |
| 12 | 1.8 | 86 |
| 13 | 3.5 | 64 |
| 14 | 0.5 | 93 |
| 15 | 272 | 100 |
| 16 | 1.2 | 92 |
| 17 | 2.8 | 92 |
| 18 | 0.8 | 78 |
| 19 | 2.4 | 93 |
| 20 | 0.5 | 91 |
| 21 | 148 | 100 |
| 22 | 0.7 | 92 |
| 23 | 0.7 | 91 |
| 24 | 0.8 | 92 |
| 25 | 0.2 | 92 |
| 26 | 0.8 | 93 |
| 27 | 0.8 | 93 |
| 28 | 5.7 | 80 |
| 29 | 1.2 | 93 |
| 30 | 0.3 | 92 |
| 31 | 0.4 | 92 |
| 32 | 0.7 | 91 |
| 33 | 0.4 | 91 |
| 34 | 12.3 | 90 |
| 35 | 0.4 | 91 |
| 36 | 0.4 | 92 |
| 37 | 0.2 | 94 |
| 38 | 0.3 | 92 |
| 39 | 0.5 | 95 |
| 40 | 0.4 | 91 |
| 41 | 0.4 | 91 |
| 42 | 0.5 | 91 |
| 43 | 0.4 | 92 |
| 44 | 0.5 | 92 |
| 45 | 0.8 | 92 |
| 46 | 2.3 | 93 |
| 47 | 145 | 100 |
| 48 | 0.8 | 92 |
| 49 | 0.8 | 92 |
| 50 | 0.7 | 91 |
| 51 | 3.4 | 92 |
| 52 | 13 | 90 |
| 53 | 0.7 | 88 |
| 54 | 0.5 | 90 |
| 55 | 1.9 | 95 |
| 56 | 7.7 | 95 |
| 57 | 0.2 | 92 |
| 58 | 0.3 | 90 |
| 59 | 0.2 | 95 |
| 60 | 1.2 | 95 |
| 61 | 4 | 90 |
| 62 | 0.6 | 95 |
| 63 | 1.6 | 90 |
| 64 | 0.7 | 100 |
| 65 | 2.5 | 100 |
| 66 | 0.2 | 90 |
| 67 | 0.4 | 93 |
| 68 | 0.3 | 92 |
| 69 | 0.2 | 91 |
| 70 | 0.4 | 92 |
| 71 | 9.7 | 96 |
| 72 | 0.5 | 91 |
| 73 | 0.3 | 84 |

It is therefore apparent that the tested compounds have degradation activities for estrogen receptors, with IC50 less than 1 μM and with degradation levels greater than 50%. The compounds of formula (I) can therefore be used for preparing medicaments, especially medicaments which are degraders of estrogen receptors.

Accordingly, also provided herein are medicaments which comprise a compound of the formula (I), or a pharmaceutically acceptable salt thereof.

Herein are also provided the compounds of formula (I) defined above, or pharmaceutically acceptable salts thereof, for use as medicines.

Herein are also provided the compounds of formula (I) defined above, or pharmaceutically acceptable salt thereof, for use in therapy, especially as inhibitors and degraders of estrogen receptors.

Herein are also provided the compounds of formula (I) defined above, or a pharmaceutically acceptable salts thereof, for use in the treatment of ovulatory dysfunction, cancer, endometriosis, osteoporosis, benign prostatic hypertrophy or inflammation.

A particular aspect is a compound of formula (I) defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor a dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound of formula (I) is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

Herein is also provided the use of a compound of the formula (I), or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound of formula (I). These pharmaceutical compositions comprise an effective dose of at least one compound of formula (I), or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle of formula (I) above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds of formula (I) in creams, gels, ointments or lotions.

As an example, a unit administration form of a compound of formula (I) in tablet form may comprise the following components:

| | |
|---|---|
| Compound of formula (I) | 50.0 mg |
| Mannitol | 223.75 mg |
| Sodium croscarmellose | 6.0 mg |
| Corn starch | 15.0 mg |
| Hydroxypropylmethylcellulose | 2.25 mg |
| Magnesium stearate | 3.0 mg |

There may be particular cases in which higher or lower dosages are appropriate. According to usual practice, the dosage that is appropriate for each patient is determined by the doctor according to the mode of administration and the weight and response of the said patient.

1. A compound of the formula (I) or a pharmaceutically acceptable salt thereof: 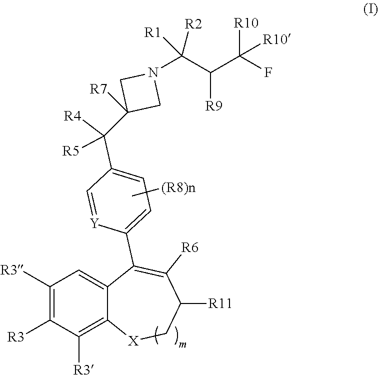
wherein:
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 and R5 independently represent a hydrogen atom, a fluorine atom, a —NH₂ group, a (C₁-C₃)alkyl group, a (C₁-C₃)alkoxy group, or a —OH group; or R4 and R5 together form an oxo group or R4 and R5 together form a ═NOCH₃ group or a (C₃-C₅)cycloalkyl group with the carbon atom to which they are attached;
R7 represents a hydrogen atom, a methyl group, a —OH group or a fluorine atom;
or alternatively R4 and R7 together form a cyclopropyl group together with the bond to which they are attached, that gives with the adjacent azetidine group an azaspiro[2.3]hexane;
R6 represents a group selected from:
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group;
a fused phenyl group, selected from phenyl groups fused with a (C₃-C₆)cycloalkyl, which (C₃-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a bicyclic group comprising 5 to 12 carbon atoms, optionally comprising 1 to 2 unsaturations; optionally substituted with 1 to 4 substituents independently selected from: a fluorine atom, a —OH group, a (C₁-C₃)-alkyl group, a (C₁-C₃)fluoroalkyl group, a (C₁-C₃)alkoxy group, a (C₁-C₃)fluoroalkoxy group and an oxo group;
a heteroaryl group comprising 2 to 9 carbon atoms and comprising from 1 to 3 heteroatoms independently selected from oxygen, nitrogen and sulfur, and at least 5 atoms including carbon atoms and heteroatoms, said heteroaryl group being optionally substituted with 1 to 3 substituents independently selected from a halogen atom, a (C₁-C₆)alkyl group, a (C₁-C₆)fluoroalkyl group, a (C₁-C₆)alkoxy group, a (C₁-C₆)fluoroalkoxy group, a cyano group, a carbamoyl group and a —OH group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
a (C₃-C₆)cycloalkyl group, and a phenyl group, said (C₃-C₆)cycloalkyl or phenyl groups being optionally substituted with one or two halogen atom(s) or (C₁-C₃)alkyl group(s);
a (C₃-C₆)cycloalkyl(C₁-C₃)alkyl group, optionally substituted on the cycloalkyl with 1 to 4 substituents independently selected from: a fluorine atom, a —OH group, a (C₁-C₄)alkyl group, a (C₁-C₃)fluoroalkyl group, a (C₁-C₃)fluoroalkoxy group and an oxo group;
a 4 to 7 membered-heterocycloalkyl group comprising 1 or 2 heteroatoms independently selected from oxygen, nitrogen and sulfur, said heterocycloalkyl group being saturated or partially saturated and being optionally substituted with 1 to 3 substituents independently selected from: a fluorine atom, a (C₁-C₃)alkyl group, a (C₁-C₃)fluoroalkyl group, a (C₁-C₃)fluoroalkoxy group, an oxo group, a (C₁-C₃)alkoxy group and a —OH group;
a (C₁-C₆)alkyl group, said alkyl group being optionally substituted with 1 to 4 substituents independently selected from: a fluorine atom, a (C₁-C₃)alkoxy group, a (C₁-C₃)fluoroalkoxy group and a —OH group; and
a phenyl(C₁-C₂)alkyl group, said phenyl group being optionally substituted with 1 to 3 substituents independently selected from a halogen atom; a (C₁-C₃)alkyl group; a (C₁-C₃)fluoroalkyl group; a (C₁-C₃)alkoxy group; a (C₁-C₃) fluoroalkoxy group; a cyano group; and a —OH group;
X represents —CH₂—, —O— or —S—;
Y represents —CH═, —N═ or —CR″=, wherein R″ represents a (C₁-C₃)alkyl group or a halogen atom, a cyano group, or a (C₁-C₃)fluoroalkyl group;
R8 independently represents a (C₁-C₃)alkyl group, a halogen atom, a cyano group, or a (C₁-C₃)fluoroalkyl group;
R9 represents a hydrogen atom or a fluorine atom;
R10 and R10′ independently represent a hydrogen atom or a fluorine atom;
R11 represents a hydrogen atom, or a (C₁-C₃)alkyl group or a cyclopropyl;
n is 0, 1 or 2, and
m is 0 or 1.

2. The compound of formula (I) according to claim 1, or a pharmaceutically acceptable salt thereof, characterized in that R1 and R2 are a hydrogen atom.

3. The compound of formula (I) according to claim 1, or a pharmaceutically acceptable salt thereof, characterized in that R3 is —COOH.

4. The compound of formula (I) according to claim 1, or a pharmaceutically acceptable salt thereof, characterized in that X represents —CH₂—.

5. The compound of formula (I) according to claim 1, or a pharmaceutically acceptable salt thereof, characterized in that R4 and R5 represent independently from each other a hydrogen atom, a fluorine atom, a methyl group, a methoxy group, an ethoxy group, a —NH₂ group or a —OH group; or R4 and R5 together form an oxo group, a=NOCH₃ group or a cyclopropyl group with the carbon atom to which they are attached or alternatively R4 and R7 together form a cyclopropyl group together with the bond to which they are attached.

6. The compound of formula (I) according to claim 1, or a pharmaceutically acceptable salt thereof, characterized in that R7 represents a hydrogen atom, a —OH group, a methyl group or a fluorine atom.

7. The compound of formula (I) according to claim 1, or a pharmaceutically acceptable salt thereof, characterized in that R6 represents a phenyl group, said phenyl group being optionally substituted with 1 to 3 substituents independently selected from a chlorine atom, a fluorine atom, a hydroxy group, a methyl group, an ethyl group, a trifluoromethyl group, a 2,2,2-trifluoroethyl group, a 1,1-difluoroethyl group, a hydroxymethyl group, a 2-hydroxyethyl group, a fluoromethyl group, a difluoromethyl group, a 2,2-difluororethyl group, a methoxy group, an ethoxy group, a cyano group, a vinyl group, a cyanomethyl group, a trifluoromethylsulfonyl group, a methylsulfanyl group, a difluoromethylsulfanyl group, a methylsulfonyl group, a trifluoromethoxy group, a cyclopropyl group, and a difluoromethoxy group.

8. The compound of formula (I) according to claim 1, or a pharmaceutically acceptable salt thereof, characterized in that R6 represents a fused phenyl group, selected from a bicyclo[4.2.0]octa-trienyl group, a tetrahydronaphthalenyl group and an indanyl group, said groups being optionally substituted with one or two fluorine atoms or R6 represents a chromanyl group.

9. The compound of formula (I) according to claim 1, or a pharmaceutically acceptable salt thereof, characterized in that R6 represents a cycloalkyl group selected from a cyclobutyl group, a cyclohexyl group, a cyclopentyl group, a cycloheptyl group, a cycloheptenyl group and a cyclohexenyl group, said cycloalkyl group being optionally substituted with 1 to 4 substituents independently selected from:
a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group,
a (C₃-C₆)cycloalkyl group and a phenyl group, said (C₃-C₆)cycloalkyl or phenyl groups being optionally substituted with one or two halogen atom(s) or a (C₁-C₃)alkyl group, said cycloalkyl being optionally substituted with 1 to 2 substituents independently selected from:
a fluorine atom, a methyl group, and
a cyclohexyl group substituted by two halogen atoms.

10. The compound of formula (I) according to claim 1, or a pharmaceutically acceptable salt thereof, characterized in that R6 represents a (C₁-C₆)alkyl group selected from an ethyl, an isobutyl group and an ethylbutyl, said alkyl group being optionally substituted with 1 to 4 substituents independently selected from: a fluorine atom, a (C₁-C₃)alkoxy group, a (C₁-C₃)fluoroalkoxy group and a —OH group.

11. The compound of formula (I) according to claim 1, or a pharmaceutically acceptable salt thereof, characterized in that R3′ and R3″ represent a hydrogen atom.

12. The compound of formula (I) according to claim 1, or a pharmaceutically acceptable salt thereof, characterized in that R8 independently represents a methyl group or a fluorine atom and n is 0, 1 or 2.

13. The compound of formula (I) according to claim 1, or a pharmaceutically acceptable salt thereof, wherein Y represents —CH═, —C(CH₃)=, —CF═ or —N═.

14. The compound of formula (I) according to claim 1, or a pharmaceutically acceptable salt thereof, wherein R9 represents a hydrogen atom.

15. The compound of formula (I) according to claim 1, or a pharmaceutically acceptable salt thereof, wherein R10 and R10′ represent a hydrogen atom.

16. The compound of formula (I) according to claim 1, or a pharmaceutically acceptable salt thereof, wherein R11 represents a hydrogen atom.

17. The compound of formula (I) according to claim 1, or a pharmaceutically acceptable salt thereof, wherein m is 1.

18. The compound of formula (I) according to claim 1, or a pharmaceutically acceptable salt thereof, wherein R6 represents
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl group is at least substituted by a (C₁-C₆)alkylene group;
a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
a (C₃-C₆)cycloalkyl group, and a phenyl group, said (C₃-C₆)cycloalkyl or phenyl groups being optionally substituted with one or two halogen atom(s) or (C₁-C₃)alkyl group(s);
wherein said cycloalkyl group is at least substituted by a (C₁-C₃)alkyl group optionally substituted with a —OH group.

19. The compound of formula (I) according to claim 1, or a pharmaceutically acceptable salt thereof, wherein R6 represents
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl is substituted by at least one hydroxy group, by at least a vinyl group, by at least a trifluoromethoxy group, by at least a cyclopropyl group or by at least a 1,1-difluoroethyl group;
a tetrahydronaphthalenyl group;
a cyclobutyl group; or
a hydroxypropyl group.

20. The compound of formula (I) according to claim 1, or a pharmaceutically acceptable salt thereof, in particular hydrochloride salt thereof, characterized in that said compound is selected from the following compounds:
8-(2-(difluoromethoxy)-3-fluorophenyl)-9-(4-((1-(3-fluoropropyl)azetidin-3-yl)methyl)phenyl)-6,7-dihydro-5H-benzo[7]annulene-3-carboxylic acid, (1)
9-(4-((1-(3-fluoropropyl)azetidin-3-yl)methyl)phenyl)-8-(5,6,7,8-tetrahydronaphthalen-1-yl)-6,7-dihydro-5H-benzo[7]annulene-3-carboxylic acid, (2)
9-(4-((1-(3-fluoropropyl)azetidin-3-yl)methyl)phenyl)-8-(3-(hydroxymethyl)cyclopentyl)-6,7-dihydro-5H-benzo[7]annulene-3-carboxylic acid, (3)
9-(4-((1-(3-fluoropropyl)azetidin-3-yl)methyl)phenyl)-8-(2-methoxy-3-(trifluoromethyl)phenyl)-6,7-dihydro-5H-benzo[7]annulene-3-carboxylic acid, (4)
8-(3-chloro-2-(trifluoromethoxy)phenyl)-9-(4-((1-(3-fluoropropyl)azetidin-3-yl)methyl)phenyl)-6,7-dihydro-5H-benzo[7]annulene-3-carboxylic acid, (5)
9-(4-((1-(3-fluoropropyl)azetidin-3-yl)methyl)phenyl)-8-(2-methyl-3-(trifluoromethoxy)phenyl)-6,7-dihydro-5H-benzo[7]annulene-3-carboxylic acid, (6)
9-(4-((1-(3-fluoropropyl)azetidin-3-yl)methyl)phenyl)-8-(3-methoxy-2-methylphenyl)-6,7-dihydro-5H-benzo[7]annulene-3-carboxylic acid, (7)
8-(2-(difluoromethyl)-3-methoxyphenyl)-9-(4-((1-(3-fluoropropyl)azetidin-3-yl)methyl)phenyl)-6,7-dihydro-5H-benzo[7]annulene-3-carboxylic acid, (8) 8-(chroman-8-yl)-9-(4-((1-(3-fluoropropyl)azetidin-3-yl)methyl)phenyl)-6,7-dihydro-5H-benzo[7]annulene-3-carboxylic acid, (9)
8-(4,4-dimethylcyclohexyl)-9-(3-fluoro-5-((1-(3-fluoropropyl)azetidin-3-yl)methyl)pyridin-2-yl)-6,7-dihydro-5H-benzo[7]annulene-3-carboxylic acid, (10)
9-(4-((1-(3-fluoropropyl)azetidin-3-yl)methyl)phenyl)-8-(3-methoxy-2-(trifluoromethyl)phenyl)-6,7-dihydro-5H-benzo[7]annulene-3-carboxylic acid, (11) 8-(chroman-5-yl)-9-(4-((1-(3-fluoropropyl)azetidin-3-yl)methyl)phenyl)-6,7-dihydro-5H-benzo[7]annulene-3-carboxylic acid, (12)
9-(5-((1-(3,3-difluoropropyl)azetidin-3-yl)methyl)-3-fluoropyridin-2-yl)-8-(4,4-dimethylcyclohexyl)-6,7-dihydro-5H-benzo[7]annulene-3-carboxylic acid, (13)
8-(3-(difluoromethoxy)-2-methylphenyl)-9-(4-((1-(3-fluoropropyl)azetidin-3-yl)methyl)phenyl)-6,7-dihydro-5H-benzo[7]annulene-3-carboxylic acid, (14)
9-(4-((1-(3-fluoropropyl)azetidin-3-yl)methyl)phenyl)-8-(3-hydroxypropyl)-6,7-dihydro-5H-benzo[7]annulene-3-carboxylic acid, 2,2,2-trifluoroacetic acid, (15)
9-(4-((1-(3-fluoropropyl)azetidin-3-yl)methyl)phenyl)-8-(2-methoxy-4-(trifluoromethyl)phenyl)-6,7-dihydro-5H-benzo[7]annulene-3-carboxylic, acid hydrochloride, (16)
9-(4-((1-(3-fluoropropyl)azetidin-3-yl)methyl)phenyl)-8-(4-methoxy-3-(trifluoromethyl)phenyl)-6,7-dihydro-5H-benzo[7]annulene-3-carboxylic acid, hydrochloride, (17)
9-(4-((1-(3-fluoropropyl)azetidin-3-yl)methyl)phenyl)-8-(2-methyl-5-(trifluoromethyl)phenyl)-6,7-dihydro-5H-benzo[7]annulene-3-carboxylic acid, (18)
9-(4-((1-(3-fluoropropyl)azetidin-3-yl)methyl)phenyl)-8-(3-methoxy-4-(trifluoromethyl)phenyl)-6,7-dihydro-5H-benzo[7]annulene-3-carboxylic acid, hydrochloride, (19)
9-(4-((1-(3-fluoropropyl)azetidin-3-yl)methyl)phenyl)-8-(5-methyl-2-(trifluoromethyl)phenyl)-6,7-dihydro-5H-benzo[7]annulene-3-carboxylic acid, hydrochloride, (20)
9-(4-((1-(3-fluoropropyl)azetidin-3-yl)methyl)phenyl)-8-(3-(hydroxymethyl)cyclobutyl)-6,7-dihydro-5H-benzo[7]annulene-3-carboxylic acid, (21)
9-(4-((1-(3-fluoropropyl)azetidin-3-yl)methyl)phenyl)-8-(3-(trifluoromethoxy)phenyl)-6,7-dihydro-5H-benzo[7]annulene-3-carboxylic acid, hydrochloride, (22)
8-(2-(difluoromethyl)-5-methylphenyl)-9-(4-((1-(3-fluoropropyl)azetidin-3-yl)methyl)phenyl)-6,7-dihydro-5H-benzo[7]annulene-3-carboxylic acid, (23)
8-(2-(difluoromethyl)-4-methoxyphenyl)-9-(4-((1-(3-fluoropropyl)azetidin-3-yl)methyl)phenyl)-6,7-dihydro-5H-benzo[7]annulene-3-carboxylic acid, (24)
9-(4-((1-(3-fluoropropyl)azetidin-3-yl)methyl)phenyl)-8-(4-methoxy-2-(trifluoromethyl)phenyl)-6,7-dihydro-5H-benzo[7]annulene-3-carboxylic acid, hydrochloride, (25)
9-(4-((1-(3-fluoropropyl)azetidin-3-yl)methyl)phenyl)-8-(3-methyl-4-(trifluoromethyl)phenyl)-6,7-dihydro-5H-benzo[7]annulene-3-carboxylic acid, (26)
9-(4-((1-(3-fluoropropyl)azetidin-3-yl)methyl)phenyl)-8-(4-(trifluoromethoxy)phenyl)-6,7-dihydro-5H-benzo[7]annulene-3-carboxylic acid, (27)
9-(4-((1-(3-fluoropropyl)azetidin-3-yl)methyl)phenyl)-8-(2-methoxy-5-(trifluoromethyl)phenyl)-6,7-dihydro-5H-benzo[7]annulene-3-carboxylic acid, hydrochloride, (28)
9-(4-((1-(3-fluoropropyl)azetidin-3-yl)methyl)phenyl)-8-(4-methyl-3-(trifluoromethyl)phenyl)-6,7-dihydro-5H-benzo[7]annulene-3-carboxylic acid, (29)
9-(4-((1-(3-fluoropropyl)azetidin-3-yl)methyl)phenyl)-8-(2-(trifluoromethoxy)phenyl)-6,7-dihydro-5H-benzo[7]annulene-3-carboxylic acid, (30)
8-(4-fluoro-2,5-dimethylphenyl)-9-(4-((1-(3-fluoropropyl)azetidin-3-yl)methyl)phenyl)-6,7-dihydro-5H-benzo[7]annulene-3-carboxylic acid, (31)
8-(2-cyclopropyl-4-(trifluoromethyl)phenyl)-9-(4-((1-(3-fluoropropyl)azetidin-3-yl)methyl)phenyl)-6,7-dihydro-5H-benzo[7]annulene-3-carboxylic acid, hydrochloride, (32)
9-(4-((1-(3-fluoropropyl)azetidin-3-yl)methyl)phenyl)-8-(2-methyl-6-(trifluoromethyl)phenyl)-6,7-dihydro-5H-benzo[7]annulene-3-carboxylic acid, (33)
9-(4-((1-(3-fluoropropyl)azetidin-3-yl)methyl)phenyl)-8-(3-methyl-5-(trifluoromethyl)phenyl)-6,7-dihydro-5H-benzo[7]annulene-3-carboxylic acid, hydrochloride, (34)
9-(4-((1-(3-fluoropropyl)azetidin-3-yl)methyl)phenyl)-8-(5-methoxy-2-(trifluoromethyl)phenyl)-6,7-dihydro-5H-benzo[7]annulene-3-carboxylic acid, (35)
8-(3-fluoro-2-vinylphenyl)-9-(4-((1-(3-fluoropropyl)azetidin-3-yl)methyl)phenyl)-6,7-dihydro-5H-benzo[7]annulene-3-carboxylic acid, (36)
8-(2-ethyl-3-fluorophenyl)-9-(4-((1-(3-fluoropropyl)azetidin-3-yl)methyl)phenyl)-6,7-dihydro-5H-benzo[7]annulene-3-carboxylic acid, (37)
8-(3-chloro-2-ethylphenyl)-9-(4-((1-(3-fluoropropyl)azetidin-3-yl)methyl)phenyl)-6,7-dihydro-5H-benzo[7]annulene-3-carboxylic acid, (38)
8-(2,4-bis(trifluoromethyl)phenyl)-9-(4-((1-(3-fluoropropyl)azetidin-3-yl)methyl)phenyl)-6,7-dihydro-5H-benzo[7]annulene-3-carboxylic acid, (39)
8-(2-chloro-6-methylphenyl)-9-(4-((1-(3-fluoropropyl)azetidin-3-yl)methyl)phenyl)-6,7-dihydro-5H-benzo[7]annulene-3-carboxylic acid, hydrochloride, (40)
8-(2-fluoro-6-methylphenyl)-9-(4-((1-(3-fluoropropyl)azetidin-3-yl)methyl)phenyl)-6,7-dihydro-5H-benzo[7]annulene-3-carboxylic acid, hydrochloride, (41)
8-(4-(difluoromethyl)-3-methylphenyl)-9-(4-((1-(3-fluoropropyl)azetidin-3-yl)methyl)phenyl)-6,7-dihydro-5H-benzo[7]annulene-3-carboxylic acid, hydrochloride, (42)
8-(2-fluoro-6-(trifluoromethoxy)phenyl)-9-(4-((1-(3-fluoropropyl)azetidin-3-yl)methyl)phenyl)-6,7-dihydro-5H-benzo[7]annulene-3-carboxylic acid, (43)
8-(2-chloro-6-methoxyphenyl)-9-(4-((1-(3-fluoropropyl)azetidin-3-yl)methyl)phenyl)-6,7-dihydro-5H-benzo[7]annulene-3-carboxylic acid, (44)
8-(2-fluoro-6-methoxyphenyl)-9-(4-((1-(3-fluoropropyl)azetidin-3-yl)methyl)phenyl)-6,7-dihydro-5H-benzo[7]annulene-3-carboxylic acid, (45)
8-(2,3-dimethoxyphenyl)-9-(4-((1-(3-fluoropropyl)azetidin-3-yl)methyl)phenyl)-6,7-dihydro-5H-benzo[7]annulene-3-carboxylic acid, (46)
8-(2,4-difluoro-3-hydroxyphenyl)-9-(4-((1-(3-fluoropropyl)azetidin-3-yl)methyl)phenyl)-6,7-dihydro-5H-benzo[7]annulene-3-carboxylic acid, (47)
9-(4-((1-(3-fluoropropyl)azetidin-3-yl)methyl)phenyl)-8-(2-methoxy-6-methylphenyl)-6,7-dihydro-5H-benzo[7]annulene-3-carboxylic acid, hydrochloride, (48)
9-(4-((1-(3-fluoropropyl)azetidin-3-yl)methyl)phenyl)-8-(2-methoxy-3-methylphenyl)-6,7-dihydro-5H-benzo[7]annulene-3-carboxylic acid, hydrochloride, (49)
8-(2-chloro-6-(trifluoromethoxy)phenyl)-9-(4-((1-(3-fluoropropyl)azetidin-3-yl)methyl)phenyl)-6,7-dihydro-5H-benzo[7]annulene-3-carboxylic acid, (50)
8-(2,6-dimethoxyphenyl)-9-(4-((1-(3-fluoropropyl)azetidin-3-yl)methyl)phenyl)-6,7-dihydro-5H-benzo[7]annulene-3-carboxylic acid, hydrochloride, (51)
9-(4-((1-(3-fluoropropyl)azetidin-3-yl)methyl)phenyl)-8-(3-hydroxy-4-(trifluoromethyl)phenyl)-6,7-dihydro-5H-benzo[7]annulene-3-carboxylic acid, (52)
8-(2-chloro-4,6-dimethylphenyl)-9-(4-((1-(3-fluoropropyl)azetidin-3-yl)methyl)phenyl)-6,7-dihydro-5H-benzo[7]annulene-3-carboxylic acid, (53)
8-(2-(difluoromethyl)-3-methylphenyl)-9-(4-((1-(3-fluoropropyl)azetidin-3-yl)methyl)phenyl)-6,7-dihydro-5H-benzo[7]annulene-3-carboxylic acid, hydrochloride, (54)
9-(4-((1-(3-fluoropropyl)azetidin-3-yl)methyl)phenyl)-8-(4-methoxy-2,6-dimethylphenyl)-6,7-dihydro-5H-benzo[7]annulene-3-carboxylic acid, (55)
9-(4-((1-(3-fluoropropyl)azetidin-3-yl)methyl)phenyl)-8-(3-hydroxy-4-methylphenyl)-6,7-dihydro-5H-benzo[7]annulene-3-carboxylic acid, (56)
8-(3-fluoro-2,4-dimethylphenyl)-9-(4-((1-(3-fluoropropyl)azetidin-3-yl)methyl)phenyl)-6,7-dihydro-5H-benzo[7]annulene-3-carboxylic acid, (57)
8-(3-chloro-2,4-dimethylphenyl)-9-(4-((1-(3-fluoropropyl)azetidin-3-yl)methyl)phenyl)-6,7-dihydro-5H-benzo[7]annulene-3-carboxylic acid, (58)
8-(2,3-bis(trifluoromethyl)phenyl)-9-(4-((1-(3-fluoropropyl)azetidin-3-yl)methyl)phenyl)-6,7-dihydro-5H-benzo[7]annulene-3-carboxylic acid, (59)
8-(2-(difluoromethyl)-5-methoxyphenyl)-9-(4-((1-(3-fluoropropyl)azetidin-3-yl)methyl)phenyl)-6,7-dihydro-5H-benzo[7]annulene-3-carboxylic acid, hydrochloride, (60)
9-(4-((1-(3-fluoropropyl)azetidin-3-yl)methyl)phenyl)-8-(3-hydroxy-2-methylphenyl)-6,7-dihydro-5H-benzo[7]annulene-3-carboxylic acid, hydrochloride, (61)
8-(2-(1,1-difluoroethyl)-4-fluorophenyl)-9-(4-((1-(3-fluoropropyl)azetidin-3-yl)methyl)phenyl)-6,7-dihydro-5H-benzo[7]annulene-3-carboxylic acid, (62)
9-(4-((1-(3-fluoropropyl)azetidin-3-yl)methyl)phenyl)-8-(2-methoxy-6-(trifluoromethyl)phenyl)-6,7-dihydro-5H-benzo[7]annulene-3-carboxylic acid, (63)
8-(3-fluoro-2-methyl-4-(trifluoromethyl)phenyl)-9-(4-((1-(3-fluoropropyl)azetidin-3-yl)methyl)phenyl)-6,7-dihydro-5H-benzo[7]annulene-3-carboxylic acid, hydrochloride, (64)
8-(3-fluoro-2-methoxy-6-methylphenyl)-9-(4-((1-(3-fluoropropyl)azetidin-3-yl)methyl)phenyl)-6,7-dihydro-5H-benzo[7]annulene-3-carboxylic acid, (65)
8-(2-(difluoromethyl)-6-methylphenyl)-9-(4-((1-(3-fluoropropyl)azetidin-3-yl)methyl)phenyl)-6,7-dihydro-5H-benzo[7]annulene-3-carboxylic acid, (66)
8-(2-ethyl-3-(trifluoromethyl)phenyl)-9-(4-((1-(3-fluoropropyl)azetidin-3-yl)methyl)phenyl)-6,7-dihydro-5H-benzo[7]annulene-3-carboxylic acid, (67)
8-(3-ethyl-2-(trifluoromethyl)phenyl)-9-(4-((1-(3-fluoropropyl)azetidin-3-yl)methyl)phenyl)-6,7-dihydro-5H-benzo[7]annulene-3-carboxylic acid, (68)
8-(2-ethyl-4-(trifluoromethyl)phenyl)-9-(4-((1-(3-fluoropropyl)azetidin-3-yl)methyl)phenyl)-6,7-dihydro-5H-benzo[7]annulene-3-carboxylic acid, (69)
8-(4-ethyl-2-(trifluoromethyl)phenyl)-9-(4-((1-(3-fluoropropyl)azetidin-3-yl)methyl)phenyl)-6,7-dihydro-5H-benzo[7]annulene-3-carboxylic acid, (70)
8-(3,4-dimethoxyphenyl)-9-(4-((1-(3-fluoropropyl)azetidin-3-yl)methyl)phenyl)-6,7-dihydro-5H-benzo[7]annulene-3-carboxylic acid, (71)
9-(4-((1-(3-fluoropropyl)azetidin-3-yl)methyl)phenyl)-8-(2-methoxy-4,6-dimethylphenyl)-6,7-dihydro-5H-benzo[7]annulene-3-carboxylic acid, (72)
8-(3-fluoro-2,6-dimethylphenyl)-9-(4-((1-(3-fluoropropyl)azetidin-3-yl)methyl)phenyl)-6,7-dihydro-5H-benzo[7]annulene-3-carboxylic acid, (73).

21. A process for preparing a compound of formula (I) as defined in claim 1, wherein a compound of formula 1G 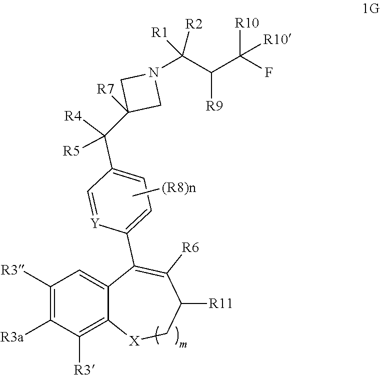
wherein R1, R2, R3′, R3″, R4, R5, R6, R7, Y, R8, R9, R10, R10′, R11, n, m, X are as defined in claim 1 and R3a is carboxylic ester or protected OH, is converted to a compound of formula (I), in the presence of a source of hydroxide ions, said step being optionally preceded by a step for obtaining compound 1G, wherein a compound of formula 1F 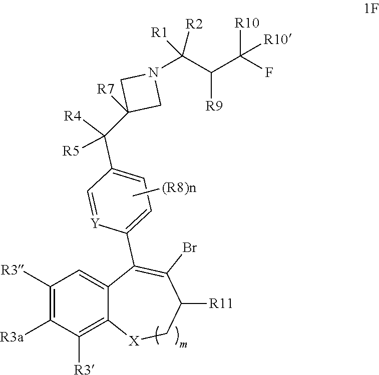
wherein, R1, R2, R3′, R3″, R4, R5, R7, Y, R8, R9, R10, R10′, R11, n, m, X are as defined in claim 1 and R3a is as defined above,
is subjected to a Suzuki coupling with a boronic reagent R6B(OR′)₂, wherein —B(OR′)₂ is a boronic acid or a pinacolate ester and R6 is as defined in claim 1.

22. A process for preparing a compound of formula (I) as defined in claim 1, wherein a compound of formula 1Fa 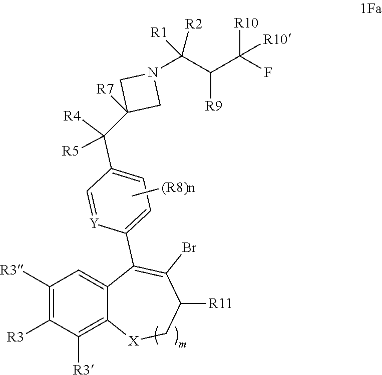
wherein R1, R2, R3, R3′, R3″, R4, R5, R7, Y, R8, R9, R10, R10′, R11, n, m, X are as defined in claim 1, is submitted to a Suzuki coupling with a boronic reagent R6B(OR′)₂, wherein —B(OR′)₂ is a boronic acid or a pinacolate ester and R6 is defined as in claim 1, said step being optionally preceded by a step for obtaining compound 1Fa, wherein a compound of formula 1F 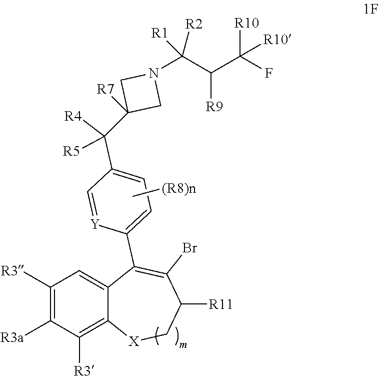
wherein R1, R2, R3′, R3″, R4, R5, R7, Y, R8, R9, R10, R10′, R11, n, m, X are as defined in claim 1 and R3a is carboxylic ester or protected OH,
is converted to a compound 1Fa in the presence of a source of hydroxide ions.

23. A compound of formula 1G, or a pharmaceutically acceptable salt thereof, 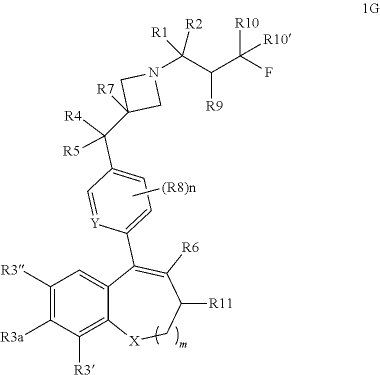
wherein
R1 and R2 independently represent a hydrogen atom or a deuterium atom;
R3 represents a hydrogen atom, a —COOH group or a —OH group;
R3′ and R3″ independently represent a hydrogen atom, a methyl group, a methoxy group, a chlorine atom, a fluorine atom, or a cyano group;
R4 and R5 independently represent a hydrogen atom, a fluorine atom, a —NH₂ group, a (C₁-C₃)alkyl group, a (C₁-C₃)alkoxy group, or a —OH group; or R4 and R5 together form an oxo group or R4 and R5 together form a ═NOCH₃ group or a (C₃-C₅)cycloalkyl group with the carbon atom to which they are attached;
R7 represents a hydrogen atom, a methyl group, a —OH group or a fluorine atom;
or alternatively R4 and R7 together form a cyclopropyl group together with the bond to which they are attached, that gives with the adjacent azetidine group an azaspiro[2.3]hexane:
X represents —CH₂—, —O— or —S—;
Y represents —CH═, —N═ or —CR″=, wherein R″ represents a (C₁-C₃)alkyl group or a halogen atom, a cyano group, or a (C₁-C₃)fluoroalkyl group;
R8 independently represents a (C₁-C₃)alkyl group, a halogen atom, a cyano group, or a (C₁-C₃)fluoroalkyl group;
R9 represents a hydrogen atom or a fluorine atom;
R10 and R10′ independently represent a hydrogen atom or a fluorine atom;
R11 represents a hydrogen atom, or a (C₁-C₃)alkyl group or a cyclopropyl;
n is 0, 1 or 2, and
m is 0 or 1; and
a phenyl group, said phenyl group being optionally substituted by 1 to 3 substituents independently selected from a halogen atom; a (C₁-C₆)alkyl group optionally substituted with a cyano group or a —OH group; a (C₁-C₆)alkylene group, a (C₁-C₆)fluoroalkyl group; a (C₃-C₆)cycloalkyl group; a (C₁-C₆)alkoxy group; a (C₁-C₆)fluoroalkoxy group; a cyano group; a trifluoromethylsulfonyl group; a (C₁-C₄)alkylthio group; a (C₁-C₄)fluoroalkylthio group; a (C₁-C₄)alkylsulfonyl group; and a —OH group, wherein said phenyl group is at least substituted by a (C₁-C₆)alkylene group;
a phenyl group fused with a hetero(C₄-C₆)cycloalkyl, which hetero(C₄-C₆)cycloalkyl ring optionally comprises an unsaturation and, wherein the fused phenyl group is optionally substituted with 1 to 3 substituents independently selected from a (C₁-C₃)alkyl group, a hydroxy group, a halogen atom, a (C₁-C₆)fluoroalkyl group and a (C₁-C₃)alkoxy group;
a cycloalkyl group comprising 3 to 7 carbon atoms, said cycloalkyl group being saturated or partially saturated and being optionally substituted with 1 to 4 substituents independently selected from:
a fluorine atom, a —OH group, a (C₁-C₃)alkyl group optionally substituted with a —OH group, a (C₁-C₃)fluoroalkyl group, a (C₁-C₃)alkoxy group, a (C₁-C₃)fluoroalkoxy group, an oxo group, and
a (C₃-C₆)cycloalkyl group, and a phenyl group, said (C₃-C₆)cycloalkyl or phenyl groups being optionally substituted with one or two halogen atom(s) or (C₁-C₃)alkyl group(s);
wherein said cycloalkyl group is at least substituted by a (C₁-C₃)alkyl group optionally substituted with a —OH group.

24. A process for preparing a compound of formula (I) as defined in claim 1, wherein a compound of formula 1Z 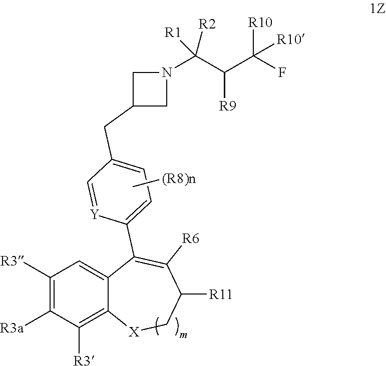
wherein R1, R2, R3′, R3″, Y, R6, R8, R9, R10, R10′, R11, n, m, X are as defined in claim 1 and R3a is carboxylic ester or protected OH, is converted into a compound of formula (I), in the presence of a source of hydroxide ions, said step being preceded by a step for obtaining a compound 1Z, wherein a compound of formula 1Y 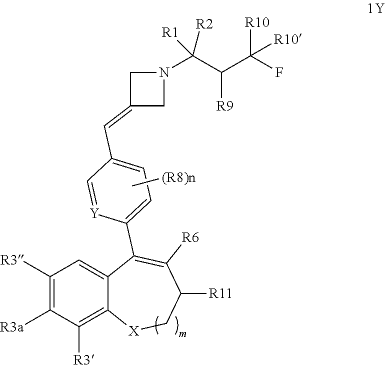
wherein R1, R2, R3, R3′, R3″, R4, R5, R6, Y, R8, R9, R10, R10′, R11, n, m, X are as defined in claim 1 and R3a is carboxylic ester or protected OH, is converted to compound 1Z by hydrogenation with a catalyst under hydrogen pressure.

25. (canceled)

26. A pharmaceutical composition, characterized in that it comprises a compound of formula (I) according to claim 1, or a pharmaceutically acceptable salt thereof, and at least one pharmaceutically acceptable excipient.

27. A method for inhibiting and degrading estrogen receptors, which method comprises administering to a subject in need thereof a therapeutically effective amount of a compound of formula (I) according claim 1, or a pharmaceutically acceptable salt thereof.

28. A method of treating ovulatory dysfunction, cancer, endometriosis, benign prostatic hypertrophy or inflammation, which method comprises administering to a subject in need thereof a therapeutically effective amount of a compound of formula (I) according to claim 1, or a pharmaceutically acceptable salt thereof.

29. The method of claim 28, which method comprises the treatment of cancer.
